(19) United States
(12) Patent Application Publication
(10) Pub. No.: US 2024/0091307 A1
(43) Pub. Date: Mar. 21, 2024

(54) COMBINATION THERAPIES OF AMUC_1100 AND IMMUNE CHECKPOINT MODULATORS FOR USE IN TREATING CANCERS

(71) Applicant: COMMBIO THERAPEUTICS CO., LTD., Shanghai (CN)

(72) Inventor: Bin Xiang, Shanghai (CN)

(21) Appl. No.: 18/267,240

(22) PCT Filed: Dec. 15, 2021
(86) PCT No.: PCT/CN2021/138210

§ 371 Date: Jun. 14, 2023

(30) Foreign Application Priority Data
Dec. 15, 2020 (WO) PCT/CN2020/136426
Jun. 2, 2021 (CN) 202110613387.8

(51) Int. Cl. A61K 38/16; A61K 39/395; A61P 35/00; C07K 16/28; C12N 15/70

(52) U.S. Cl. CPC A61K 38/164; A61K 39/3955; A61P 35/00; C07K 16/2818; C12N 15/70; C12N 2800/101

(57) ABSTRACT

The present disclosure provides a composition comprising Amuc_1100 (or a genetically engineered host cell expressing the Amuc_1100) and an immune checkpoint modulator, and uses thereof.

### FIELD OF THE INVENTION

The present disclosure generally relates to the fields of cancer treatment and immunity improvement.

### BACKGROUND

Immune checkpoint therapy is a breakthrough therapy in cancer treatment. Immune checkpoints comprise inhibitory and stimulatory pathways that maintain self-tolerance and assist with immune response. Currently, there are several immune checkpoint inhibitor drugs approved for treating cancer patients, and more are of such kind of therapeutics are under development (Andrews, L. P. et al., Nat Immunol, 2019. 20 (11): p. 1425-1434). Furthermore, agonists of stimulatory checkpoint pathways such as OX40, ICOS, GITR, 4-1BB, CD40, or molecules targeting tumor microenvironment components like IDO or TLR are under investigation (Julian A. Marin-Acevedo et al., Journal of Hematology & Oncology, 11, 39 (2018)). However, the efficacy of immune checkpoint modulator drugs varies among cancer types. For example, the response rates to anti-PD-1 antibodies range from 40% to 70% in some cancers (melanoma, microsatellite instability MSI-high tumors) while the response rate is limited to 10% to 25% in most other cancers or even to <10% in other cancers (triple negative breast cancer, microsatellite stable (MSS) colorectal cancer, etc.) (Schoenfeld, A. J. et al., Cancer Cell, 2020. 37 (4): p. 443-455.; Ciardiello, D. et al., Cancer Treat Rev, 2019. 76: p. 22-32; Adams, S. et al., JAMA Oncol, 2019). Furthermore, disease progression can eventually develop among patients who initially respond to cancer immunotherapy. Overall, only small population of cancer patients can have long-term benefit from cancer immunotherapy. Although there have been over one thousand of clinical studies exploring the combination therapy strategy for the approved or underdevelopment immune checkpoint modulators in cancers, none has been proven to be highly effective. In particular, it is a big challenge to develop an efficient and synergetic combination therapy with immune checkpoint inhibitors to achieve long-term and durable response in cancer (Xin Yu, J. et al., Nat Rev Drug Discov, 2020. 19 (3): p. 163-164).

Recent evidences suggest that gut microbiota composition especially certain bacteria strains can predict or increase the efficacy of cancer immunotherapy. Inoculation of Akkermansia muciniphila into antibiotic-treated specific-pathogen-free mice significantly enhanced anti-PD-1 therapeutic efficacy in treating melanoma (Routy, B. et al., Science, 2018. 359 (6371): p. 91-97). However, it is unclear which component(s) of the gut microbiota composition exert the function of enhancing anti-PD-1 therapeutic efficacy.

Needs remain for novel therapy for treating cancers.

### SUMMARY OF THE INVENTION

In one aspect, the present disclosure provides a method of treating or preventing cancer in a subject in need thereof, comprising: a) administering to the subject an effective amount of a gut immunity modulator capable of modulating gut immunity; and b) administering to the subject an effective amount of an immune checkpoint modulator.

In another aspect, the present disclosure provides a method of improving therapeutic response to cancer in a subject receiving treatment of an immune checkpoint modulator, comprising administering to the subject an effective amount of gut immunity modulator, optionally in combination with an effective amount of an immune checkpoint modulator.

In another aspect, the present disclosure provides a method of inducing or improving gut immunity in a subject, comprising administering to the subject an effective amount of gut immunity modulator, in combination with an effective amount of an immune checkpoint modulator.

In another aspect, the present disclosure provides a method of improving therapeutic response to cancer in a subject receiving an anti-cancer treatment and showing decrease in gut immunity, comprising administering to the subject an effective amount of gut immunity modulator.

In another aspect, the present disclosure provides a method of treating or preventing cancer in a subject in need thereof, comprising administering to the subject an effective amount of Amuc_1100 or a genetically engineered host cell expressing the Amuc_1100, in combination with an effective amount of an immune checkpoint modulator.

In another aspect, the present disclosure provides a method of improving therapeutic response to cancer in a subject receiving treatment of an immune checkpoint modulator, comprising administering to the subject an effective amount of Amuc_1100 or a genetically engineered host cell expressing the Amuc_1100, optionally in combination with an effective amount of an immune checkpoint modulator.

In another aspect, the present disclosure provides a method of inducing or improving gut immunity in a subject, comprising administering to the subject an effective amount of Amuc_1100 or a genetically engineered host cell expressing the Amuc_1100, in combination with an effective amount of an immune checkpoint modulator.

In another aspect, the present disclosure provides a method of improving therapeutic response to cancer in a subject receiving an anti-cancer treatment and showing decrease in gut immunity, comprising administering to the subject an effective amount of Amuc_1100 or a genetically engineered host cell expressing the Amuc_1100.

In certain embodiments, the Amuc_1100 is a naturally-occurring Amuc_1100, or a functional equivalent thereof. In certain embodiments, the functional equivalent retains at least partial activity in modulating gut immunity and/or in activating toll-like receptor 2 (TLR2). In certain embodiments, the functional equivalent comprises a mutant, a fragment, a fusion, a derivative, an equivalent that improves the stability thereof, or any combination thereof of the naturally-occurring Amuc_1100. In certain embodiments, the Amuc_1100 comprises an amino acid sequence selected from the group consisting of SEQ ID NO: 1, SEQ ID NO: 2 and SEQ ID NO: 3, or an amino acid sequence having at least 80% sequence identity thereof yet retaining substantial activity in modulating gut immunity and/or in activating toll-like receptor 2 (TLR2).

In certain embodiments, the Amuc_1100 comprises one or more mutations at a position selected from the group consisting of: R36, S37, L39, D40, K41, K42, 143, K48, E49, K51, S52, R62, S63, K70, E71, L72, N73, R74, Y75, A76, K77, A78, Y86, K87, P88, F89, L90, A91, F103, Q104, K108, T109, F110, R111, D112, K119, K120, K121, N122, L124, 1125, W131, L132, G133, F134, Q135, Y137, S138, L150, G151, F152, E153, L154, K155, A156, L160, V161, K163, L164, A165, L169, S170, K171, F172, I173, K174, V175, Y176, R177, W200, T201, L205, E206, F209, Q210, R213, E214, L217, K218, A219, M220, N221, Y229, L230, R237, I238, R242, M243, M244, P245, K255, P256, L268, T269, K287, P288, Y289, M290, K292, E293, F296, V297, F306, N307, K310 and A311, wherein the numbering is relative to SEQ ID NO: 1. In certain embodiments, the Amuc_1100 comprises one or more mutations at a position selected from the group consisting of: S37, V175, Y289 and F296, wherein the numbering is relative to SEQ ID NO: 1. In certain embodiments, the Amuc_1100 comprises Y289A mutation, wherein the numbering is relative to SEQ ID NO: 1.

In certain embodiments, the immune checkpoint modulator is an antibody or an antigen-binding fragment thereof or a chemical compound against an immune checkpoint molecule. In certain embodiments, the immune checkpoint modulator comprises an activator of one or more immunostimulatory checkpoints selected from the group consisting of: CD2, CD3, CD7, CD16, CD27, CD30, CD70, CD83, CD28, CD80 (B7-1), CD86 (B7-2), CD40, CD40L (CD154), CD47, CD122, CD137, CD137L, OX40 (CD134), OX40L (CD252), NKG2C, 4-1BB, LIGHT, PVRIG, SLAMF7, HVEM, BAFFR, ICAM-1, 2B4, LFA-1, GITR, ICOS (CD278), ICOSLG (CD275), and any combination thereof. In certain embodiments, the immune checkpoint modulator comprises an inhibitor of one or more immunoinhibitory checkpoints selected from the group consisting of LAG3 (CD223), A2AR, B7-H3 (CD276), B7-H4 (VTCN1), BTLA (CD272), BTLA, CD160, CTLA-4 (CD152), IDO1, IDO2, TDO, KIR, LAIR-1, NOX2, PD-1, PD-L1, PD-L2, TIM-3, VISTA, SIGLEC-7 (CD328), TIGIT, PVR (CD155), TGF β, SIGLEC9 (CD329), and any combination thereof. In certain embodiments, the immune checkpoint modulator is an inhibitor of one or more immunoinhibitory checkpoints, preferably an antibody or an antigen-binding fragment thereof or a chemical compound that inhibits or reduces interaction between PD-1 and any of its ligands. In certain embodiments, the immune checkpoint modulator is an antibody or an antigen-binding fragment thereof or a chemical compound against PD-L1, PD-L2, or PD-1.

In certain embodiments, the subject has shown poor response, or resistance to the immune checkpoint modulator. In certain embodiments, the subject is determined to have de novo or acquired resistance to the immune checkpoint modulator.

In certain embodiments, the subject is a cancer patient, wherein the cancer is selected from the group consisting of colorectal cancer, breast cancer, ovarian cancer, pancreatic cancer, gastric cancer, prostate cancer, renal cancer, cervical cancer, myeloma cancer, lymphoma cancer, leukemia cancer, thyroid cancer, endometrial cancer, uterine cancer, bladder cancer, neuroendocrine cancer, head and neck cancer, liver cancer, nasopharyngeal cancer, testicular cancer, small cell lung cancer, non-small cell lung cancer, melanoma, basal cell cancer, skin cancer, squamous cell skin cancer, dermatofibrosarcoma protuberans, Merkel cell carcinoma, glioblastoma, glioma, sarcoma and mesothelioma.

In certain embodiments, the administration of the Amuc_1100 or genetically engineered host cell expressing the Amuc_1100 is via oral administration. In certain embodiments, the administration of the Amuc_1100 or genetically engineered host cell expressing the Amuc_1100 is before, after, or simultaneously with the immune checkpoint modulator.

In certain embodiments, the host cell comprises a probiotic microorganism or a non-pathogenic microorganism. In certain embodiments, the host cell comprises an exogenous expression cassette comprising a nucleotide sequence that encodes Amuc_1100 operably linked to a signal peptide, optionally, wherein the genetically engineered host cell secrets at least 10 ng Amuc_1100 from 1×10⁹ CFU of the genetically engineered host cell.

In another aspect, the present disclosure provides a genetically engineered host cell comprising an exogenous expression cassette comprising a nucleotide sequence that encodes Amuc_1100 operably linked to a signal peptide, optionally, wherein the genetically engineered host cell secrets at least 10 ng Amuc_1100 from 1×10⁹ CFU of the genetically engineered host cell.

In another aspect, the present disclosure provides a recombinant expression cassette comprising a nucleotide sequence that encodes Amuc_1100 operably linked to a signal peptide, and one or more regulatory elements.

In certain embodiments, the host cell comprises a probiotic microorganism or a non-pathogenic microorganism. In certain embodiments, the exogenous expression cassette is integrated in a plasmid of the genetically engineered host cell. In certain embodiments, the exogenous expression cassette is integrated in the genome of the genetically engineered host cell.

In certain embodiments, the exogenous expression cassette comprises a nucleotide sequence that encodes Amuc_1100 operably linked to a signal peptide, wherein the signal peptide is operably linked at the N-terminus of the Amuc_1100. In certain embodiments, the signal peptide comprises an amino acid sequence selected from the group consisting of SEQ ID NOs: 76-82 and homologous sequences thereof having at least 80% sequence identity. In certain embodiments, the signal peptide is Usp45 signal peptide. In certain embodiments, the signal peptide can be processed by a secretion system present in the host cell. In certain embodiments, the secretion system is native or non-native to the host cell. In certain embodiments, the host cell is probiotic bacteria. In certain embodiments, the secretion system is engineered and/or optimized such that at least one outer membrane protein encoding gene is deleted, inactivated, or suppressed. In certain embodiments, the outer membrane protein is selected from the group consisting of: OmpC, OmpA, OmpF, OmpT, pldA, pagP, tolA, Pal, To1B, degS, mrcA and 1pp. In certain embodiments, the secretion system is engineered such that at least one Chaperone protein encoding gene is amplified, overexpressed or activated. In certain embodiments, the Chaperone protein is selected from the group consisting of: dsbA, dsbC, dnaK, dnaJ, grpE, groES, groEL, tig, fkpA, surA, skp, PpiD and DegP.

In certain embodiments, the expression cassette further comprises one or more regulatory elements comprises one or more elements selected from the group consisting of: a promoter, a ribosome binding site (RBS), a cistron, a terminator, and any combination thereof. In certain embodiments, the promoter is a constitutive promoter, or an inducible promoter.

In certain embodiments, the promoter is an endogenous promoter, or an exogenous promoter. In certain embodiments, the constitutive promoter comprises a nucleotide sequence selected from the group consisting of SEQ ID NOs: 14-54 and homologous sequences thereof having at least 80% sequence identity. In certain embodiments, the constitutive promoter comprises SEQ ID NO: 15. In certain embodiments, the inducible promoter comprises a nucleotide sequence selected from the group consisting of SEQ ID NOs: 55-58 and homologous sequences thereof having at least 80% sequence identity. In certain embodiments, the inducible promoter comprises SEQ ID NO: 58.

In certain embodiments, the RBS comprises a nucleotide sequence selected from the group consisting of SEQ ID NOs: 70-73 and homologous sequences thereof having at least 80% sequence identity. In certain embodiments, the cistron comprises a nucleotide sequence selected from the group consisting of SEQ ID NOs: 66-69 and homologous sequences thereof having at least 80% sequence identity. In certain embodiments, the terminator is T7 terminator. In certain embodiments, the promoter is rrnB_T 1_T7Te terminator. In certain embodiments, the promoter comprises a nucleotide sequence as shown in SEQ ID NO: 74.

In certain embodiments, the genetically engineered host cell of the present disclosure further comprises at least one inactivation or deletion in an auxotroph-related gene. In certain embodiments, the auxotroph-related gene is selected from the group consisting of thyA, cysE, glnA, ilvD, leuB, lysA, serA, metA, glyA, hisB, ilvA, pheA, proA, thrC, trpC, tyrA, uraA, dapF, flhD, metB, metC, proAB, yhbV, yagG, hemB, secD, secF, ribD, ribE, thiL, dxs, ispA, dnaX, adk, hemH, IpxH, cysS, fold, rp1T, infC, thrS, nadE, gapA, yeaZ, aspS, argS, pgsA, yeflA, metG, folE, yejM, gyrA, nrdA, nrdB, folC, accD, fabB, gltX, ligA, zipA, dapE, dapA, der, hisS, ispG, suhB, tadA, acpS, era, rnc, fisB, eno, pyrG, chpR, Igt, ft>aA, pgk, yqgD, metK, yqgF, plsC, ygiT, pare, ribB, cca, ygjD, tdcF, yraL, yihA, ftsN, murl, murB, birA, secE, nusG, rp1J, rp1L, rpoB, rpoC, ubiA, plsB, lexA, dnaB, ssb, alsK, groS, psd, orn, yjeE, rpsR, chpS, ppa, valS, yjgP, yjgQ, dnaC, ribF, IspA, ispH, dapB, folA, imp, yabQ, flsL, fisl, murE, murF, mraY, murD, ftsW, murG, murC, ftsQ, ftsA, ftsZ, IpxC, secM, secA, can, folK, hemL, yadR, dapD, map, rpsB, in/B,nusA, ftsH, obgE, rpmA, rplU, ispB, murA, yrbB, yrbK, yhbN, rpsl, rplM, degS, mreD, mreC, mreB, accB, accC, yrdC, def, fint, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yr/F, asd, rpoH, ftsX, ftsE, ftsY, frr, dxr, ispU, rfaK, kdtA, coaD, rpmB, djp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, IpxA, IpxB, dnaE, accA, tilS, proS, yafF, tsf, pyrH, olA, rlpB, leuS, Int, glnS, fidA, cydA, in/A, cydC, ftsK, lolA, serS, rpsA, msbA, IpxK, kdsB, mukF, mukE, mukB, asnS, fabA, mviN, rne, yceQ, fabD, fabG, acpP, tmk, holB, lo1C, loiD, lolE, purB, ymflC, minE, mind, pth, rsA, ispE, l1iB, hemA, prfA, prmC, kdsA, topA, ribA, fabi, racR, dicA, yd B, tyrS, ribC, ydiL, pheT, pheS, yhhQ, bcsB, glyQ, yibJ, and gpsA.

In certain embodiments, the host cell of the present disclosure is an auxotroph for one or more substances selected from the group consisting of uracil, leucine, histidine, tryptophan, lysine, methionine, adenine, and non-naturally occurring amino acid. In certain embodiments, the non-naturally occurring amino acid is selected from the group consisting of 1-4,4′-biphenylalanine, p-acetyl-1-phenylalanine, p-iodo-1-pheylalanine, and p-azido-1-phenylalanine.

In certain embodiments, the host cell of the present disclosure comprises an allosterically regulated transcription factor which is capable of detecting a signal in an environment that modulates activity of the transcription factor, wherein absence of the signal would lead to the cell death.

In certain embodiments, the probiotic microorganism of the present disclosure is a probiotic bacterium or a probiotic yeast. In certain embodiments, the probiotic bacterium is selected from the group consisting of Bacteroides, Bifidobacterium, Clostridium, Escherichia, Lactobacillus and Lactococcus, optionally, the probiotic bacterium is of the genus Escherichia, and optionally, the probiotic bacterium is of the species Escherichia coli strain Nissle 1917 (EcN). In certain embodiments, the probiotic yeast is selected from the group consisting of Saccharomyces cerevisiae, Candida utilis, Kluyveromyces lactis, and Saccharomyces carlsbergensis.

In another aspect, the present disclosure provides a composition comprising the genetically engineered host cell of the present disclosure, and a physiologically acceptable carrier.

In another aspect, the present disclosure provides a composition comprising: a) the genetically engineered host cell of the present disclosure or an isolated Amuc 1100; and b) an immune check point inhibitor.

In certain embodiments, the composition of the present disclosure is edible. In certain embodiments, the composition is a probiotic composition.

In another aspect, the present disclosure provides a kit comprising: a) a first composition comprising the genetically engineered host cell of the present disclosure or comprising an isolated Amuc_1100; and b) a second composition comprising an immune check point inhibitor.

In certain embodiments, the first composition is an edible composition, and/or the second composition further comprises a pharmaceutically acceptable carrier. In certain embodiments, the first composition is a food supplement. In certain embodiments, the second composition is suitable for oral administration or parenteral administration.

In another aspect, the present disclosure provides a genetically engineered host cell of the present disclosure for use in combination with an immune checkpoint modulator.

In another aspect, the present disclosure provides a nucleic acid vehicle comprising the recombinant expression cassette of the present disclosure for use in combination with an immune checkpoint modulator.

In another aspect, the present disclosure provides an oral composition comprising a genetically engineered host cell of any one of the present disclosure or an isolated Amuc_1100 for use in combination with a formulation comprising an immune checkpoint modulator.

In certain embodiments, the formulation comprising an immune checkpoint modulator is a parenteral formulation. In certain embodiments, the formulation comprising an immune checkpoint modulator is an oral formulation.

In another aspect, the present disclosure provides a non-naturally occurring Amuc_1100 protein, wherein the Amuc_100 protein comprises Y289A mutation, wherein the numbering is relative to SEQ ID NO: 1.

In another aspect, the present disclosure also provides a nucleic acid encoding the Amuc_1100 protein provided herein.

In another aspect, the present disclosure provides use of Usp45 signal peptide in the construction of an expression vector comprising a polynucleotide encoding a target polypeptide, wherein the expression vector is suitable for expression in E. Coli to allow expression and secretion of the target polypeptide from the E. Coli.

In another aspect, the present disclosure provides Usp45 signal peptide, for use in the construction of an expression vector comprising a polynucleotide encoding a target polypeptide, wherein the expression vector is suitable for expression in E. Coli to allow expression and secretion of the target polypeptide from the E. Coli.

In another aspect, the present disclosure provides an expression vector comprising a polynucleotide encoding a target polypeptide operably linked to Usp45 signal peptide, wherein the expression vector is suitable for expression in E. Coli to allow expression and secretion of the target polypeptide from the E. Coli.

In certain embodiments, the Usp45 signal peptide comprises the sequence as set forth in SEQ ID NO: 59.

In certain embodiments, the E. Coli is a commensal strain of E. Coli.

In certain embodiments, the commensal strain is E. coli Nissle 1917 strain.

In certain embodiments, the target polypeptide comprises Amuc_1100.

In certain embodiments, the Amuc_1100 is a naturally-occurring Amuc_1100, or a functional equivalent thereof.

In certain embodiments, the target polypeptide is a polypeptide other than Amuc_1100.

In another aspect, the present disclosure provides a genetically engineered E. coli, comprising an exogenous expression cassette comprising polynucleotide encoding a target polypeptide operably linked to Usp45 signal peptide.

In certain embodiments, the genetically engineered E. coli provided herein is capable of expressing and secreting the target polypeptide.

### BRIEF DESCRIPTION OF THE DRAWINGS

FIG. 1 shows the plasmid profile of plasmid pET-22b (+)_Amuc_1100.

FIG. 2 shows the sequences of SEQ ID NOs: 145-147.

FIG. 3A shows the stability assay results of Amuc_1100 (31-317, Y289A) mutant protein. Recombinant Amuc_1100 wild type (WT) and mutant protein are digested by proteases trypsin (0.01 mg/ml) and α-chymotrypsin (0.01 mg/ml) at 25° C. for 30 min. The samples are subjected to SDS-PAGE gel electrophoresis and Coomassie blue staining. Lane A: Amuc_1100 (31-317, Y289A) without proteases; Lane B: Amuc_1100 (31-317, Y289A) with proteases; Lane C: Amuc_1100 (31-317, WT) without proteases; Lane D: Amuc_1100 (31-317, WT) with proteases; MW: molecular weight marker (KD).)

FIG. 3B shows the cell viability assay results of Amuc_1100 (31-317), Amuc_1100 (31-317, Y289A) and Pam3CSK4 (positive control).

FIG. 4 shows the H&E staining results to determine the tumor cells in the tissues treated by PBS (negative control), Amuc_1100 (31-317), Amuc_1100 (31-317, Y289A), anti-PD-1 antibody, combination of Amuc_1100 (31-317) and anti-PD-1 antibody, combination of Amuc_1100 (31-317, Y289A) and anti-PD-1 antibody, respectively.

FIG. 5 shows the tumor growth rate of each mouse treated by PBS (negative control), Amuc_1100 (31-317), Amuc_1100 (31-317, Y289A), anti-PD-1 antibody, combination of Amuc_1100 (31-317) and anti-PD-1 antibody, combination of Amuc_1100 (31-317, Y289A) and anti-PD-1 antibody, respectively.

FIG. 6 shows the plasmid profile of plasmid pCBT003_agaI/rsmI.

FIG. 7 shows the plasmid profile of plasmid pUC57_Amuc_1100.

FIG. 8 shows the plasmid profile of plasmid pCBT001.

FIG. 9 shows a schematic drawing illustrating the integration of PCR fragment of Amuc_1100 (31-317) expression cassette into the agaI/rsmI site of EcN using CRISPR-Cas9.

FIG. 10 shows the plasmid profile of plasmid pCBT003_agaI/rsmI_sgRNA.

FIG. 11 shows a schematic drawing illustrating the insertion of cistron(s) into the Amuc_1100 (31-317) expression cassette.

FIG. 12A shows a schematic drawing illustrating the insertion of autotransporter domain(s) into the Amuc_1100 expression cassette.

FIG. 12B shows the western blot result for Amuc_1100 secretion with different signal peptides in bacteria transfected with plasmid vectors.

FIG. 13A shows the secretion of Amuc_1100 from the engineered bacteria, wherein LANE A and LANE B: CBT1101 having only one copy of Amuc_1100 (Y289A) under the control of the promoter BBA_J23101; LANE C: CBT1102 having only one copy of Amuc_1100 (Y289A) under the control of the promoter BBA_J23110; LANE D: CBT1103 having two copies of Amuc_1100 (Y289A) under the control of the promoter BBA_J23110; LANE E: CBT1104 having three copies of Amuc_1100 (Y289A) under the control of the promoter BBA_J23101; LANE F: CBT1105 having two copies of Amuc_1100 (Y289A) under the control of the promoter BBA_J23101, and one copy of Amuc_1100 (Y289A) under the control of the promoter BBA_J23110; LANE G: CBT1106 having three copies of Amuc_100 (Y289A) under the control of the promoter BBA_J23110.

FIG. 13B shows secretion of Amuc_1100 from CBT1107 having one copy of anaerobic promoter under aerobic and anaerobic conditions.

FIG. 13C shows secretion of Amuc_1100 from CBT1108 having two copies of anaerobic promoters under aerobic and anaerobic conditions.

FIG. 13D shows secretion of Amuc_1100 from engineered bacteria having different promoter combinations under aerobic conditions.

FIG. 13E shows secretion of Amuc_1100 from engineered bacteria having different promoter combinations under anaerobic conditions.

FIG. 13F shows growth of the engineered bacteria having different copy numbers of anaerobic promoters under culture conditions having aerobic to anaerobic transition.

FIG. 13G shows the effect of the Lpp gene knockout on the secretion of Amuc_1100 protein.

FIG. 13H shows the effect of the ompT gene knockout on the secretion of Amuc_1100 protein.

FIG. 13I shows the expression of Amuc_1100 from the CBT1116 strain or the CBT1103 strain.

FIG. 14 shows a schematic drawing illustrating the integration of Amuc_1100 expression cassette into suitable sites (e.g. agaI/rsmI) of the EcN genome.

FIG. 15 shows the map of the integration sites.

FIG. 16 shows an example of one genetically engineered and optimized EcN that encoding Amuc_1100 gene cassette.

FIG. 17 shows the TLR2 reporter activity results of secreted Amuc_100 (31-317) from genetically engineered EcN.

FIG. 18 shows the tumor growth rate of each mouse that has been treated with EcN without genetic modification (negative control), genetically engineered bacteria encoding two copies of Amuc_1100 gene cassettes, anti-PD-1 antibody, or combination of genetically engineered bacteria encoding two copies of Amuc_1100 gene cassettes and anti-PD-1 antibody.

FIG. 19 shows the nucleic acid sequence of pCBT003.

FIG. 20 shows the nucleic acid sequence of pCBT001.

### DETAILED DESCRIPTION OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entireties.

L Definitions

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., JMolBiol. Dec 5;186(3):651-63 (1985); Chothia, C. and Lesk, A. M., J.Mol.Biol., 196,901 (1987); Chothia, C. et al., Nature. Dec 21-28;342(6252):877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gammal heavy chain), IgG2 (gamma2 heavy chain), IgG3 (gamma3 heavy chain), IgG4 (gamma4 heavy chain), IgA1 (alphal heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragments include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among amino acid residues with neutral hydrophilic side chains (e.g. Cys, Ser, Thr, Asn and Gln), among amino acid residues with acidic side chains (e.g. Asp, Glu), among amino acid residues with basic side chains (e.g. His, Lys, and Arg), or among amino acid residues with aromatic side chains (e.g. Trp, Tyr, and Phe). As known in the art, conservative substitution usually does not cause significant change in the protein conformational structure, and therefore could retain the biological activity of a protein.

The term “effective amount” or “pharmaceutically effective amount” as used herein refers to the amount and/or dosage, and/or dosage regime of one or more agents necessary to bring about the desired results, e.g., an amount sufficient to mitigate in a subject one or more symptoms associated with the disease condition for which the subject is receiving therapy, or an amount sufficient to lessen the severity or delay the progression of the disease condition in a subject (e.g., therapeutically effective amounts), an amount sufficient to reduce the risk or delaying the onset, and/or reduce the ultimate severity of a disease condition in a subject (e.g., prophylactically effective amounts).

The term “encoded” or “encoding” as used herein means capable of transcription into mRNA and/or translation into a peptide or protein. The term “encoding sequence” or “gene” refers to a polynucleotide sequence encoding a peptide or protein. These two terms can be used interchangeably in the present disclosure. In some embodiments, the encoding sequence is a complementary DNA (cDNA) sequence that is reversely transcribed from a messenger RNA (mRNA). In some embodiments, the encoding sequence is mRNA.

The term “homologous” as used herein refers to nucleic acid sequences (or its complementary strand) or amino acid sequences that have sequence identity of at least 60% (e.g. at least 65%, 70%, 75%, 80%, 85%, 88%, 90%, 91%, 92%, 93%, 94%, 95%, 96%, 97%, 98%, 99%) to another sequences when optimally aligned.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated” nucleic acid sequence refers to the sequence of an isolated nucleic acid molecule. An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purpose of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the peptides, polypeptides or proteins. Proteins or peptides expressed in most bacterial cultures will typically be free of glycan. Fragments, derivatives, analogs or variants of the foregoing peptides, polypeptides, proteins, and any combination thereof are also included as peptides, polypeptides and proteins.

The term “operably link” or “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The terms “polypeptide”, “peptide” and “protein” are used interchangeably herein to refer to a polymer of amino acid residues. The terms also apply to amino acid polymers in which one or more amino acid residue is an artificial chemical mimetic of a corresponding naturally-occurring amino acid, as well as to naturally-occurring amino acid polymers and non-naturally occurring amino acid polymers.

The term “nucleotide sequence”, “nucleic acid” or “polynucleotide” as used herein includes oligonucleotides (i.e., short polynucleotides). They also refer to synthetic and/or non-naturally occurring nucleic acid molecules (e.g., comprising nucleotide analogues or modified backbone residues or linkages). The terms also refer to deoxyribonucleotide or ribonucleotide oligonucleotides in either single-or double-stranded form. The terms encompass nucleic acids containing analogues of natural nucleotides. The terms also encompass nucleic acid-like structures with synthetic backbones. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

The term “probiotics” as used herein refers to microbial cell preparations (such as, living microbial cells) which, when administered in an effective amount, provide a beneficial effect on the health or well-being of a subject. By definition, all probiotics have a proven non-pathogenic character. In one embodiment, these health benefits are associated with improving the balance of human or animal microbiota, and/or for restoring a normal microbiota.

The term “synergistic” or “synergistically” as used herein refers to two or more agents providing a therapeutic effect that is greater than the sum of the therapeutic effects of the two or more agents provided as therapy alone.

The term “subject” as used herein includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Different vectors can be suitable for different host cells. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector.

II. Methods of Treatment, Recombinant Expression Cassettes, and Genetically Engineered Host Cells

It is well known in the art that tumors evolve during their initiation and progression to evade destruction by the immune system. While the recent use of immune checkpoint modulators to reverse this resistance has demonstrated some success, only small population of cancer patients can have long-term benefit from cancer immunotherapy.

In one aspect, the present disclosure provides a method of treating or preventing cancer in a subject in need thereof, comprising: a) administering to the subject an effective amount of a gut immunity modulator capable of modulating gut immunity; and b) administering to the subject an effective amount of an immune checkpoint modulator.

In another aspect, the present disclosure provides a method of improving therapeutic response to cancer in a subject receiving treatment of an immune checkpoint modulator, comprising administering to the subject an effective amount of gut immunity modulator, optionally in combination with an effective amount of an immune checkpoint modulator.

In another aspect, the present disclosure provides a method of inducing or improving gut immunity in a subject, comprising administering to the subject an effective amount of gut immunity modulator, in combination with an effective amount of an immune checkpoint modulator.

In another aspect, the present disclosure provides a method of improving therapeutic response to cancer in a subject receiving an anti-cancer treatment and showing decrease in gut immunity, comprising administering to the subject an effective amount of gut immunity modulator.

As used herein, the term “gut immunity modulator” refers to a biological substance that can modulate and/or promote the gut immune system function, and/or maintain, restore, or increase the physical integrity of the gut mucosal barrier in a mammal (e.g. human). In some embodiments, the gut immunity modulator is capable of modulating the activity of a toll-like receptor 2 (TLR2). In some embodiments, the gut immunity modulator is capable of activating TLR2.

TLR2 plays an essential role in detecting a diverse range of microbial pathogen-associated molecular patterns from bacteria, fungi, and parasites. TLR2 forms heterodimers with TLR1 and TLR6 on cell surface in intestinal epithelial cells and immune cells. Activation of TLR2 dimer triggers signal cascade to initiate diverse innate and adaptive immune response in the host. In addition, TLR2 activation increase intestinal epithelial cell-cell junction and therefore enhance gut barrier.

In certain embodiments, the gut immunity modulator comprises Amuc_1100.

In addition, the present inventors unexpectedly found that Amuc_1100 and functional equivalents thereof, can synergize with immune checkpoint modulator(s), such that the therapeutic effects of immune checkpoint modulator(s) can be improved or enhanced, and/or resistance to immune checkpoint modulator(s) can be reduced, delayed or prevented. Indeed, the present inventors demonstrated that combination of Amuc_1100 (e.g. purified recombinant Amuc_1100 protein or a genetically engineered host cell expressing the Amuc_1100) and an immune checkpoint modulator (e.g. anti-PD-1 antibody) exhibited synergistic anti-tumor effect beyond what was observed with the respective monotherapies, for example, in inhibiting tumor growth.

In one aspect, the present disclosure provides a method of treating or preventing cancer in a subject in need thereof, comprising administering to the subject an effective amount of Amuc_1100 or a genetically engineered host cell expressing the Amuc_1100, in combination with an effective amount of an immune checkpoint modulator.

In another aspect, the present disclosure provides a method of improving therapeutic response to cancer in a subject receiving treatment of an immune checkpoint modulator, comprising administering to the subject an effective amount of Amuc_1100 or a genetically engineered host cell expressing the Amuc_1100, optionally in combination with an effective amount of an immune checkpoint modulator.

In another aspect, the present disclosure provides a method of inducing or improving gut immunity in a subject, comprising administering to the subject an effective amount of Amuc_1100 or a genetically engineered host cell expressing the Amuc_1100, in combination with an effective amount of an immune checkpoint modulator.

In another aspect, the present disclosure provides a method of improving therapeutic response to cancer in a subject receiving an anti-cancer treatment and showing decrease in gut immunity, comprising administering to the subject an effective amount of Amuc_1100 or a genetically engineered host cell expressing the Amuc_1100.

A. Amuc 1100

Amuc_1100 is known as an outer-membrane protein which is conserved in Akkermansia muciniphila (“A. muciniphila”) bacteria genus. Along with two other members, Amuc_1099 and Amuc_1101, Amuc_1100 is located within the gene cluster involved in type IV pilus-like formation (Ottman et al., PLoS One, 2017). Exemplary amino acid sequence of Amuc_1100 is disclosed in GenBank: ACD04926.1. The terms “Amuc_1100” and “AMUC_1100” are used interchangeably in the present disclosure.

The term “Amuc_1100” as used herein broadly encompasses Amuc_1100 polypeptide, as well as Amuc_1100 polynucleotide such as a DNA or RNA sequence encoding the Amuc_1100 polypeptide. The term “Amuc_1100” as used herein further encompasses all functional equivalents of a naturally-occurring Amuc_1100. The term “functional equivalent” as used herein with respect to Amuc_1100 means any Amuc_1100 variant that, despite of having difference in amino acid sequences or polynucleotide sequences or in chemical structures, retains at least partially, one or more biological functions of naturally-occurring Amuc_1100. The biological function of naturally-occurring Amuc_1100 include, without limitation, a) modulating and/or promoting gut immune system function in a mammal, b) maintaining, restoring, and/or increasing the physical integrity of the gut mucosal barrier in a mammal, c) activating TLR2, d) improving immune response to a cancer immunotherapy (e.g. an immune checkpoint modulator) in a mammal, and e) reducing, delaying, and/or preventing resistance to immune checkpoint modulator(s) in a mammal.

In certain embodiments, a functional equivalent of Amuc_1100 retains substantial biological activity of the parent molecule. In certain embodiments, a functional equivalent of Amuc_1100 described herein still retain substantially similar functions as the naturally-occurring Amuc_1100, for example, the functional equivalent of Amuc_1100 may retain at least partial (e.g. at least 80%, 85%, 90%, 91%, 92%, 93%, 94%, 95%, 96%, 97%, 98%, 99% or 100%) of the naturally-occurring Amuc_1100's activity, for example, in modulating gut immunity and/or in activating TLR2.

The term “variant” with respect to Amuc_1100 as used herein encompasses all kinds of different forms of Amuc_1100, including without limitation, fragments, mutants, fusions, derivatives, mimetics, or any combination thereof, of a naturally-occurring Amuc_1100.

The term “mutant” as used herein refers to a polypeptide or a polynucleotide having at least 70% sequence identity to a parent sequence. A mutant may differ from the parent sequence by one or more amino acid residues or one or more nucleotides. For example, a mutant may have substitutions (including but not limited to conservative substitutions), additions, deletions, insertions, or truncations, or any combination thereof, of one more amino acid residue or one or more nucleotides of the parent sequence. In certain embodiments, a mutant Amuc_1100 has at least 75%, 80%, 85%, 90%, 91%, 92%, 93%, 94%, 95%, 96%, 97%, 98%, 99% or more sequence identity to a naturally-occurring counterpart.

The term “fragment” as used herein refers to partial sequence of the parent polypeptide or parent polynucleotide of any length. A fragment can still retain at least partial function of the parent sequence.

The term “fusion” or “fused” when used with respect to amino acid sequences (e.g. peptide, polypeptide or protein) refers to combination of two or more amino acid sequences, for example by chemical bonding or recombinant means, into a single amino acid sequence which does not exist naturally. A fusion amino acid sequence may be produced by genetic recombination of two encoding polynucleotide sequences, and can be expressed by a method of introducing a construct containing the recombinant polynucleotides into a host cell.

The term “derivative” as used herein with respect to a polypeptide or a polynucleotide refers to a chemically modified polypeptide or polynucleotide, in which one or more well-defined number of substituent groups have been covalently attached to one or more specific amino acid residues of the polypeptide or one or more specific nucleotides of the polynucleotide. Exemplary chemical modification to polypeptide can be, e.g. alkylation, acylation, esterification, amidation, phosphorylation, glycosylation, labeling, methylation of one or more amino acids, or conjugation with one or more moieties. Exemplary chemical modification to polynucleotide can be (a) end modifications, e.g., 5′ end modifications or 3′ end modifications, (b) nucleobase (or “base”) modifications, including replacement or removal of bases, (c) sugar modifications, including modifications at the 2′, 3′, and/or 4′ positions, and (d) backbone modifications, including modification or replacement of the phosphodiester linkages.

The term “naturally-occurring” as used herein with respect to Amuc_1100, means that the sequence of Amuc_1100 polypeptide or polynucleotide is identical to that or those found in nature. A naturally-occurring Amuc_1100 can be a native or wild-type sequence of Amuc_1100, or a fragment thereof, even if the fragment itself may not be found in nature. A naturally-occurring Amuc_1100 can also include a naturally-occurring variant such as mutants or isoforms or different native sequences found in different bacteria strains. A naturally-occurring full-length Amuc_1100 polypeptide has a length of 317 amino acid residues. Exemplary amino acid sequences of naturally-occurring Amuc_100 include, without limitation, Amuc_1100 (1-317) (SEQ ID NO: 1), Amuc_1100 (31-317) (SEQ ID NO: 2), and Amuc_1100 (81-317) (SEQ ID NO: 3). While not wishing to be bound by any theory, certain naturally-occurring Amuc_1100 fragments (e.g. Amuc_1100 (31-317) (SEQ ID NO: 2), and Amuc_1100 (81-317) (SEQ ID NO: 3)) may bind and activate TLR2 at a higher affinity than the full length counterpart.

In certain embodiments, Amuc_1100 comprises an Amuc_1100 polypeptide. In some embodiments, the Amuc_100 polypeptide comprises or is a naturally-occurring Amuc_1100. In some embodiments, the naturally-occurring Amuc_1100 has an amino acid sequence of SEQ ID NO: 1, SEQ ID NO: 2, or SEQ ID NO: 3.

In some embodiments, the Amuc_1100 polypeptide comprises a functional equivalent of a naturally-occurring Amuc_1100 polypeptide.

The functional equivalent of Amuc_1100 polypeptide may comprise a mutant, a fragment, a fusion, a derivative, an equivalent that improves the stability thereof, or any combination thereof of the naturally-occurring Amuc_1100. The functional equivalent of Amuc_1100 may include artificially-produced variants of the naturally-occurring Amuc_1100, such as artificial polypeptide sequences obtained by recombinant methods or chemical synthesis. The functional equivalent of Amuc_1100 may comprise non-naturally occurring amino acid residues, on the premises that the activity is not affected. Suitable non-natural amino acids include, for example, β-fluoroalanine, 1-methylhistidine, γ-methylene glutamate, α-methyl leucine, 4,5-dehydrolysine, hydroxyproline, 3-fluorophenylalanine, 3-aminotyrosine, 4-methyltryptophan, etc.

In certain embodiments, the Amuc_1100 polypeptides comprises one or more (e.g. 2, 3, 4, 5, 6, 7, 8, 9, 10, 11, 12, 13, 14, 15, 16, 17, 18, 19, 20, 21, 22, 23, 24, 25 or more) mutations at a position selected from the group consisting of: R36, S37, L39, D40, K41, K42, 143, K48, E49, K51, S52, R62, S63, K70, E71, L72, N73, R74, Y75, A76, K77, A78, Y86, K87, P88, F89, L90, A91, F103, Q104, K108, T109, F110, R111, D112, K119, K120, K121, N122, L124, I125, W131, L132, G133, F134, Q135, Y137, 5138, L150, G151, F152, E153, L154, K155, A156, L160, V161, K163, L164, A165, L169, 5170, K171, F172, 1173, K174, V175, Y176, R177, W200, T201, L205, E206, F209, Q210, R213, E214, L217, K218, A219, M220, N221, Y229, L230, R237, I238, R242, M243, M244, P245, K255, P256, L268, T269, K287, P288, Y289, M290, K292, E293, F296, V297, F306, N307, K310 and A311, wherein the numbering is relative to SEQ ID NO: 1. Without wishing to be bound by any theory, it is believed that the mutations may be useful to improve stability of Amuc_1100 polypeptide, such that it is less prone to enzymatic digestion in comparison with naturally-occurring counterparts.

In certain embodiments, the Amuc_1100 polypeptide comprises one or more (e.g. 2, 3, or 4) mutations at a position selected from the group consisting of: S37, V175, Y289 and F296, wherein the numbering is relative to SEQ ID NO: 1. In certain embodiments, the Amuc_1100 polypeptide comprises Y289A mutation, wherein the numbering is relative to SEQ ID NO: 1. In certain embodiments, the Amuc_100 polypeptide comprises an amino acid sequence of SEQ ID NO: 4 or SEQ ID NO: 5.

In certain embodiments, the Amuc_1100 polypeptide further comprises a tag or an amino acid extension at either N-terminus or C-terminus. Tag can be useful for purification of the Amuc_1100 polypeptide. An amino acid extension may be useful in improving stability or reducing clearance. Any suitable tags or extensions can be used, for example a His-tag (e.g. 6×His tag), protein A, lacZ (β-gal), maltose binding protein (MBP), calmodulin binding peptide (CBP), intein-chitin binding domain (intein-CBD) tag, streptavidin/biotin-based tag, tandem affinity purification (TAP) tag, epitope tag, reporter tag, etc.

In certain embodiments, the Amuc_1100 polypeptide further comprises an enzyme digestion site separating the tag from the rest of the Amuc_1100 polypeptide. The enzyme digestion site can be useful to remove the tag when desired. Examples of suitable enzyme digestion sites include an enterokinase recognition site (e.g. DDDDK), a factor Xa recognition site, a genenase I recognition site, a furin recognition site, etc.

Exemplary amino acid sequences of Amuc_1100 polypeptides, as well as Amuc_1100 polypeptides comprising a tag and/or an enzyme digestion site, are provided in SEQ TD NOs: 1-13, as shown in Table 1 below.

TABLE 1
Amino acid sequences of exemplary Amuc_1100

| SEQ ID NO. | Name/Description | Amino Acid Sequence |
|---|---|---|
| 1 | Amuc_1100 | MSNWITDNKPAAMVAGVGLLLFLGLSATG |
| | (1-317) | YIVNSKRSELDKKISIAAKEIKSANAAEITPS |
| | | RSSNEELEKELNRYAKAVGSLETAYKPFLA |
| | | SSALVPTTPTAFQNELKTFRDSLISSCKKKN |
| | | ILITDTSSWLGFQVYSTQAPSVQAASTLGFE |
| | | LKAINSLVNKLAECGLSKFIKVYRPQLPIET |
| | | PANNPEESDEADQAPWTPMPLEIAFQGDRE |
| | | SVLKAMNAITGMQDYLFTVNSIRIRNERM |
| | | MPPPIANPAAAKPAAAQPATGAASLTPADE |
| | | AAAPAAPAIQQVIKPYMGKEQVFVQVSLN |
| | | LVHFNQPKAQEPSED |
| 2 | Amuc_1100 | MIVNSKRSELDKKISIAAKEIKSANAAEITPS |
| | (31-317) | RSSNEELEKELNRYAKAVGSLETAYKPFLA |
| | | SSALVPTTPTAFQNELKTFRDSLISSCKKKN |
| | | ILITDTSSWLGFQVYSTQAPSVQAASTLGFE |
| | | LKAINSLVNKLAECGLSKFIKVYRPQLPIET |
| | | PANNPEESDEADQAPWTPMPLEIAFQGDRE |
| | | SVLKAMNAITGMQDYLFTVNSIRIRNERM |
| | | MPPPIANPAAAKPAAAQPATGAASLTPADE |
| | | AAAPAAPAIQQVIKPYMGKEQVFVQVSLN |
| | | LVHFNQPKAQEPSED |
| 3 | Amuc_1100 | MSLETAYKPFLASSALVPTTPTAFQNELKT |
| | (81-317) | FRDSLISSCKKKNILITDTSSWLGFQVYSTQ |
| | | APSVQAASTLGFELKAINSLVNKLAECGLS |
| | | KFIKVYRPQLPIETPANNPEESDEADQAPW |
| | | TPMPLEIAFQGDRESVLKAMNAITGMQDY |
| | | LFTVNSIRIRNERMMPPPIANPAAAKPAAA |
| | | QPATGAASLTPADEAAAPAAPAIQQVIKPY |
| | | MGKEQVFVQVSLNLVHFNQPKAQEPSED |
| 4 | Amuc_1100 | MIVNSKRSELDKKISIAAKEIKSANAAEITPS |
| | (31-317, Y289A) | RSSNEELEKELNRYAKAVGSLETAYKPFLA |
| | | SSALVPTTPTAFQNELKTFRDSLISSCKKKN |
| | | ILITDTSSWLGFQVYSTQAPSVQAASTLGFE |
| | | LKAINSLVNKLAECGLSKFIKVYRPQLPIET |
| | | PANNPEESDEADQAPWTPMPLEIAFQGDRE |
| | | SVLKAMNAITGMQDYLFTVNSIRIRNERM |
| | | MPPPIANPAAAKPAAAQPATGAASLTPADE |
| | | AAAPAAPAIQQVIKPAMGKEQVFVQVSLN |
| | | LVHFNQPKAQEPSED |
| 5 | Amuc_1100 | MSLETAYKPFLASSALVPTTPTAFQNELKT |
| | (81-317, Y289A) | FRDSLISSCKKKNILITDTSSWLGFQVYSTQ |
| | | APSVQAASTLGFELKAINSLVNKLAECGLS |
| | | KFIKVYRPQLPIETPANNPEESDEADQAPW |
| | | TPMPLEIAFQGDRESVLKAMNAITGMQDY |
| | | LFTVNSIRIRNERMMPPPIANPAAAKPAAA |
| | | QPATGAASLTPADEAAAPAAPAIQQVIKPA |
| | | MGKEQVFVQVSLNLVHFNQPKAQEPSED |
| 6 | Amuc_1100 | MIVNSKRSELDKKISIAAKEIKSANAAEITPS |
| | (31-317) with 6 x | RSSNEELEKELNRYAKAVGSLETAYKPFLA |
| | his tag at | SSALVPTTPTAFQNELKTFRDSLISSCKKKN |
| | C-terminus | ILITDTSSWLGFQVYSTQAPSVQAASTLGFE |
| | | LKAINSLVNKLAECGLSKFIKVYRPQLPIET |
| | | PANNPEESDEADQAPWTPMPLEIAFQGDRE |
| | | SVLKAMNAITGMQDYLFTVNSIRIRNERM |
| | | MPPPIANPAAAKPAAAQPATGAASLTPADE |
| | | AAAPAAPAIQQVIKPYMGKEQVFVQVSLN |
| | | LVHFNQPKAQEPSEDHHHHHH |
| 7 | Amuc_1100 | MSLETAYKPFLASSALVPTTPTAFQNELKT |
| | (81-317) with 6 x | FRDSLISSCKKKNILITDTSSWLGFQVYSTQ |
| | his tag at | APSVQAASTLGFELKAINSLVNKLAECGLS |
| | C-terminus | KFIKVYRPQLPIETPANNPEESDEADQAPW |
| | | TPMPLEIAFQGDRESVLKAMNAITGMQDY |
| | | LFTVNSIRIRNERMMPPPIANPAAAKPAAA |
| | | QPATGAASLTPADEAAAPAAPAIQQVIKPY |
| | | MGKEQVFVQVSLNLVHFNQPKAQEPSEDH |
| | | HHHHH |
| 8 | Amuc_1100 | MIVNSKRSELDKKISIAAKEIKSANAAEITPS |
| | (31-317, Y289A) | RSSNEELEKELNRYAKAVGSLETAYKPFLA |
| | with 6 x his tag at | SSALVPTTPTAFQNELKTFRDSLISSCKKKN |
| | C-terminus | ILITDTSSWLGFQVYSTQAPSVQAASTLGFE |
| | | LKAINSLVNKLAECGLSKFIKVYRPQLPIET |
| | | PANNPEESDEADQAPWTPMPLEIAFQGDRE |
| | | SVLKAMNAITGMQDYLFTVNSIRIRNERM |
| | | MPPPIANPAAAKPAAAQPATGAASLTPADE |
| | | AAAPAAPAIQQVIKPAMGKEQVFVQVSLN |
| | | LVHFNQPKAQEPSEDHHHHHH |
| 9 | Amuc_1100 | MSLETAYKPFLASSALVPTTPTAFQNELKT |
| | (81-317, Y289A) | FRDSLISSCKKKNILITDTSSWLGFQVYSTQ |
| | with 6 x his tag at | APSVQAASTLGFELKAINSLVNKLAECGLS |
| | C-terminus | KFIKVYRPQLPIETPANNPEESDEADQAPW |
| | | TPMPLEIAFQGDRESVLKAMNAITGMQDY |
| | | LFTVNSIRIRNERMMPPPIANPAAAKPAAA |
| | | QPATGAASLTPADEAAAPAAPAIQQVIKPA |
| | | MGKEQVFVQVSLNLVHFNQPKAQEPSEDH |
| | | HHHHH |
| 10 | Amuc_1100 | MHHHHHHDDDDKIVNSKRSELDKKISIAA |
| | (31-317) with 6 x | KEIKSANAAEITPSRSSNEELEKELNRYAKA |
| | his tag and | VGSLETAYKPFLASSALVPTTPTAFQNELK |
| | enterokinase | TFRDSLISSCKKKNILITDTSSWLGFQVYST |
| | recognition site at | QAPSVQAASTLGFELKAINSLVNKLAECGL |
| | N-terminus | SKFIKVYRPQLPIETPANNPEESDEADQAP |
| | | WTPMPLEIAFQGDRESVLKAMNAITGMQD |
| | | YLFTVNSIRIRNERMMPPPIANPAAAKPAA |
| | | AQPATGAASLTPADEAAAPAAPAIQQVIKP |
| | | YMGKEQVFVQVSLNLVHFNQPKAQEPSED |
| 11 | Amuc_1100 | MHHHHHHDDDDKSLETAYKPFLASSALVP |
| | (81-317) with 6 x | TTPTAFQNELKTFRDSLISSCKKKNILITDTS |
| | his tag and | SWLGFQVYSTQAPSVQAASTLGFELKAINS |
| | enterokinase | LVNKLAECGLSKFIKVYRPQLPIETPANNPE |
| | recognition site at | ESDEADQAPWTPMPLEIAFQGDRESVLKA |
| | N-terminus | MNAITGMQDYLFTVNSIRIRNERMMPPPIA |
| | | NPAAAKPAAAQPATGAASLTPADEAAAPA |
| | | APAIQQVIKPYMGKEQVFVQVSLNL VHFN |
| | | QPKAQEPSED |
| 12 | Amuc_1100 | MHHHHHHDDDDKIVNSKRSELDKKISIAA |
| | (31-317, Y289A) | KEIKSANAAEITPSRSSNEELEKELNRYAKA |
| | with 6 x his tag and | VGSLETAYKPFLASSALVPTTPTAFQNELK |
| | enterokinase | TFRDSLISSCKKKNILITDTSSWLGFQVYST |
| | recognition site at | QAPSVQAASTLGFELKAINSLVNKLAECGL |
| | N-terminus | SKFIKVYRPQLPIETPANNPEESDEADQAP |
| | | WTPMPLEIAFQGDRESVLKAMNAITGMQD |
| | | YLFTVNSIRIRNERMMPPPIANPAAAKPAA |
| | | AQPATGAASLTPADEAAAPAAPAIQQVIKP |
| | | AMGKEQVFVQVSLNLVHFNQPKAQEPSED |
| 13 | Amuc_1100 | MHHHHHHDDDDKSLETAYKPFLASSALVP |
| | (81-317, Y289A) | TTPTAFQNELKTFRDSLISSCKKKNILITDTS |
| | with 6 x his tag and | SWLGFQVYSTQAPSVQAASTLGFELKAINS |
| | enterokinase | LVNKLAECGLSKFIKVYRPQLPIETPANNPE |
| | recognition site at | ESDEADQAPWTPMPLEIAFQGDRESVLKA |
| | N-terminus | MNAITGMQDYLFTVNSIRIRNERMMPPPIA |
| | | NPAAAKPAAAQPATGAASLTPADEAAAPA |
| | | APAIQQVIKPAMGKEQVFVQVSLNLVHFN |
| | | QPKAQEPSED |

A person having ordinary skill in the art would understand that, the methionine (M) at the N-terminus of the amino acid sequence of Amuc_110 in Table 1 is encoded by a start codon, and will be removed by enzymes during protein maturation. In some embodiments, the amino acid sequence of the aforementioned Amuc_1100 may not include the N-terminal methionine (M). However, the codon encoding methionine (M) is necessary in the process of translation. Therefore, in the embodiment of the genetically engineered bacteria, the coding sequence of Amuc_1100 includes the codon encoding methionine (M).

The Amuc_1100 polypeptide provided herein can be produced by various well-known methods in the art, for example, isolation and purification from natural materials, recombinant expression, chemical synthesis, etc. The produced Amuc_1100 polypeptide can be further purified by well-known purification methods in the art.

The Amuc_1100 polynucleotide can include a polynucleotide encoding any of the Amuc_1100 polypeptides provided herein.

B. Genetically Engineered Host Cells

In certain embodiments, the methods provided herein comprises administering to the subject an effective amount of the genetically engineered host cells expressing the Amuc_1100 provided herein. It is also an aspect of the present disclosure to provide a genetically engineered host cell comprising an exogenous expression cassette comprising a nucleotide sequence that encodes Amuc_1100 operably linked to a signal peptide, wherein the genetically engineered host cell secrets at least 20 ng, at least 15 ng, at least 10 ng, at least 9 ng, at least 8 ng, at least 7 ng, at least 6 ng, at least 5 ng, at least 4 ng, at least 3 ng, at least 2 ng, at least 1 ng Amuc_1100 from 1×10⁹ CFU of the genetically engineered host cell.

In certain embodiments, the genetically engineered host cells comprises an exogenous expression cassette comprising a nucleotide sequence that encodes Amuc_1100 operably linked to a signal peptide, wherein the genetically engineered host cell secrets at least 20 ng, at least 15 ng, at least 10 ng, at least 9 ng, at least 8 ng, at least 7 ng, at least 6 ng, at least 5 ng, at least 4 ng, at least 3 ng, at least 2 ng, at least 1 ng Amuc_1100 from 1×10⁹ CFU of the genetically engineered host cell.

Genetically engineered host cells (e.g. genetically engineered bacterium) are a new class of therapeutics under exploration in drug development. In some embodiments, disease-curing gene(s) are integrated into the bacterial genome and the live engineered bacteria serve as delivery system to produce gene drug(s) at the site of the body for example the small intestine or colon where regulating the pathogenic signal. It is called as bacterial vector gene therapy or recombinant live biotherapeutic products (LBP). It is the new approach in both fields of the human microbiota and synthetic biology in developing novel class of medicine for human diseases. The present inventors unexpectedly found that the genetically engineered host cells used in the present invention achieved significantly enhanced expression and secretion of Amuc_1100 compared to the amount of Amuc_1100 produced in nature A. muciniphila. In some embodiments, the expression level of Amuc_1100 produced by the genetically engineered host cells used in the present disclosure is at least 10%, 15%, 20%, 30%, 40%, 50%, 60%, 70%, 80%, 90% and 100% higher than that of Amuc_1100 produced by nature A. muciniphila. In addition, some existing research data indicate that A. muciniphila has positive and negative effects on the host's physiological conditions. Although studies support that the use of live or inactivated A. muciniphila can increase the response rate of cancer to PD-1 antibodies, the enhancement conferred by Amuc_1100 protein or genetically engineered bacteria expressing Amuc_1100 should be safer and more specific.

Although the term “genetic engineering” is frequently used to describe a large number of techniques for the artificial modification of the genetic information of an organism, throughout the specification and claims it is employed only in reference to the in vitro technique of forming recombinant DNA's from a donor microorganism and a suitable vector, selecting on the basis of the desired genetic information, and introducing the selected DNA into a suitable microorganism (host microorganism) whereby the desired (foreign) genetic information becomes part of the genetic complement of the host. The term “genetically engineered host cell”, as used throughout the specification and claims, means a host cell prepared by this technique.

The term “host cell” as used herein refers to the cells in which the exogenous expression cassette (including a vector comprising an expression cassette) is introduced in a manner to allow expression of the polynucleotides encoding the Amuc_1100. A host cell also includes any progeny of the subject host cell or its derivatives. It is understood that all progenies may not be identical to the parental cell since there may be mutations that occur during replication. However, such progenies are included when the term “host cell” is used. Host cells, which are useful in the invention, include bacterial cells, fungal cells (e.g., yeast cells), plant cells or animal cells. In certain embodiment, the host cells are lower eukaryotic cell, such as a yeast cell, or the host cell can be a prokaryotic cell, such as a bacterial cell. Introduction of the expression cassette into the host cell can be effected by calcium phosphate transfection, DEAE-Dextran mediated transfection, or electroporation (Davis, L., Dibner, M., Battey, I., Basic Methods in Molecular Biology (1986)).

In some embodiments, the host cell of the present disclosure comprises a probiotic microorganism. The term “microorganism” as used herein includes bacteria, archaea, yeast, algae and filamentous fungi. In some embodiments, the microorganism used in the present invention is non-pathogenic microorganism. “Non-pathogenic microorganism” refer to microorganisms that are not capable of causing disease or harmful responses in a host. In some embodiments, non-pathogenic microorganisms do not contain lipopolysaccharides (LPS). In some embodiments, non-pathogenic microorganisms are commensal bacteria. In some embodiments, non-pathogenic microorganisms are attenuated pathogenic bacteria.

In some embodiments, the genetically engineered host cells (e.g. bacteria) are Gram-negative bacteria. The genetically engineered host cells (e.g. bacteria) are Gram-positive bacteria. Exemplary bacteria include, but are not limited to, Bacillus (e.g. Bacillus coagulans, Bacillus subtilis), Bacteroides (e.g. Bacteroides fragilis, Bacteroides subtilis, Bacteroides thetaiotaomicron), Bifidobacterium (e.g. Bifidobacterium adolescentis, Bifidobacterium bifidum, Bifidobacterium breve UCC2003, Bifidobacterium infantis, Bifidobacterium lactis, Bifidobacterium longum), Brevibacteria, Caulobacter, Clostridium (e.g. Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NY, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum), Corynebacterium parvum, Enterococcus, Escherichia coli (e.g. Escherichia coli MG1655, Escherichia coli Nissle 1917), Lactobacillus, Lactococcus, Listeria, Mycobacterium (e.g. Mycobacterium bovis), Saccharomyces, Salmonella (Salmonella choleraesuis, Salmonella typhimurium), Staphylococcus, Streptococcus, Vibrio (e.g. Vibrio cholera). In certain embodiments, the genetically engineered bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In some embodiments, the genetically engineered bacteria are E. coli.

A “probiotic microorganism” refers to a microorganism that can provide health benefits when consumed. Probiotic microorganisms (also known as “probiotics”) are commonly consumed as part of fermented foods with specially added active live cultures, such as in yogurt, soy yogurt, or as dietary supplements. Generally, probiotics help gut microbiota keep (or re-find) its balance, integrity and diversity. The effects of probiotics can be strain-dependent. In the context of the present invention, a “probiotic composition” is thus not limited to food or food supplements, but it generally designates any bacterial composition comprising microorganisms which are beneficial to the patients. Such probiotic compositions can hence be medicaments or drugs.

In some embodiments, the probiotic microorganism is a probiotic bacterium or a probiotic yeast. In some embodiments, the probiotic bacterium is selected from the group consisting of Bacteroides, Bifidobacterium (e.g. Bifidobacterium bifidum), Clostridium, Escherichia, Lactobacillus (e.g. Lactobacillus acidophilus, Lactobacillus bulgaricus, Lactobacillus paracasei, Lactobacillus plantarum) and Lactococcus, optionally, the probiotic bacterium is of the genus Escherichia, and optionally, the probiotic bacterium is of the species Escherichia coli strain Nissle 1917 (EcN). In some embodiments, the probiotic yeast is selected from the group consisting of Saccharomyces cerevisiae, Candida utilis, Kluyveromyces lactis, and Saccharomyces carlsbergensis. In some embodiments, the probiotic microorganism is Escherichia coli strain Nissle 1917 (EcN).

In some embodiments, the exogenous expression cassette is integrated into a plasmid of the genetically engineered host cell. A “plasmid” means a circular or linear DNA molecule that is substantially smaller than a chromosome, separate from the chromosome or chromosomes of a host cell, and that replicates separately from the chromosome or chromosomes. A “plasmid” can be present in about one copy per cell or in more than one copy per cell. Maintenance of a plasmid within a host cell in general requires an antibiotic selection, but complementation of an auxotroph can also be used.

In some embodiments, the exogenous expression cassette is integrated into the genome of the genetically engineered host cell. In some embodiments, the exogenous expression cassette is integrated into the genome of the genetically engineered host cell by CRISPR-Cas genome editing system. Selected genome sites are tested and used to integrate Amuc_1100 gene cassette. Series of modification on the gene cassette and bacterial genome will be conducted to ensure the heterogeneous gene will be expressed at certain amount and will have no major negative impact on the chassis host cell's biochemical and physiological activity.

i. Expression Cassette

The genetically engineered host cells expressing the Amuc_1100 provided herein comprises an exogenous expression cassette comprising a polynucleotide sequence that encodes the Amuc_1100 polypeptide provided herein, optionally operably linked to a signal peptide and one or more regulatory elements. It is also an aspect of the present disclosure to provide such an exogenous expression cassette comprising a polynucleotide sequence that encodes the Amuc_1100 polypeptide provided herein.

The term “expression cassette” as used herein refers to a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a non-translated RNA, in the sense of antisense direction. The expression cassette comprising the nucleotide sequence of interest may be chimeric, meaning that at least one of its components is heterologous with respect to at least one of its other components.

The expression cassette is suitable for expressing the Amuc_1100 polypeptide in the host cell provided herein. The expression cassette may be used in the form of a naked nucleic acid construct. Alternatively, the expression cassette may be introduced as part of a nucleic acid vector (e.g. an expression vector such as those described above). Suitable vectors for host cells such as bacterial cells, fungal cells (e.g., yeast cells), plant cells or animal cells can include plasmids and viral vectors. A vector may include sequences flanking the expression cassette that include sequences homologous to eukaryotic genomic sequences, such as mammalian genomic sequences, or viral genomic sequences. This will allow the introduction of the expression cassette into the genome of eukaryotic cells or viruses by homologous recombination.

The term “recombinant” as used herein refers to a polynucleotide synthesized or otherwise manipulated in vitro (e.g., “recombinant polynucleotide” or “recombinant expression cassette”), to methods of using recombinant polynucleotides or recombinant expression cassette to produce products in cells or other biological systems, or to a polypeptide (“recombinant protein”) encoded by a recombinant polynucleotide. Recombinant polynucleotides encompass nucleic acid molecules from different sources ligated into an expression cassette or vector for expression of, e.g., a fusion protein; or those produced by inducible or constitutive expression of a polypeptide (e.g., an expression cassette or vector of the invention operably linked to a heterologous polynucleotide, such as an Amuc_1100 coding sequence). Recombinant expression cassette encompasses a recombinant polynucleotide operably linked to one or more regulatory elements.

In some embodiments, the expression cassette comprises one or more regulatory elements that comprise one or more elements selected from the group consisting of: a promoter, a ribosome binding site (RBS), a cistron, a terminator, and any combination thereof.

The term “promoter” as used herein refers to an untranslated DNA sequence typically upstream of a coding region that contains the binding site for RNA polymerase and initiates transcription of the DNA. The promoter region may also include other elements that act as regulators of gene expression. In some embodiments, the promoter is suitable for initiation of the polynucleotide encoding the Amuc_1100 polypeptide in the genetically engineered host cell. In some embodiments, the promoter is a constitutive promoter, or an inducible promoter.

The term “constitutive promoter” refers to a promoter that is capable of facilitating continuous transcription of a coding sequence or gene under its control and/or to which it is operably linked. Constitutive promoters and variants are well known in the art and include, but are not limited to, BBa_J23119, BBa_J23101, BBa_J23102, BBa_J23103, BBa_J23109, BBa_J23110, BBa_J23114, BBa_J23117, USP45_promoter, Amuc_1102_promoter, OmpA_promoter, BBa_J23100, BBa_J23104, BBa_J23105, BBa_114018, BBa_J45992, BBa_J23118, BBa_J23116, BBa_J23115, BBa_J23113, BBa_J23112, BBa_J23111, BBa_J23108, BBa_J23107, BBa_J23106, BBa 114033, BBa_K256002, BBa_K1330002, BBa_J44002, BBa_J23150, BBa_I14034, Oxbl9, oxb20, BBa_K088007, Ptet, Ptrc, PlacUV5, BBa_K292001, BBa_K292000, BBa_K137031, and BBa_K137029. The nucleotide sequences of exemplary constitutive promoters comprise a nucleotide sequence selected from the group consisting of SEQ ID NOs: 14-54 as shown in Table 2, and homologous sequences thereof having at least 80% (e.g. at least 85%, 90%, 91%, 92%, 93%, 94%, 95%, 96%, 97%, 98%, 99%) sequence identity. In some embodiments, the constitutive promoter comprises the nucleotide sequence of SEQ ID NO: 15. In some embodiments, such promoters are active in vitro, e.g., under culture, expansion and/or manufacture conditions. In some embodiments, such promoters are active in vivo, e.g., in conditions found in the in vivo environment, e.g., the gut and/or the tumor microenvironment.

The term “inducible promoter” as used herein refers to a regulated promoter that can be turned on in one or more cell types by an external stimulus, such as a chemical, light, hormone, stress, or a pathogen. Inducible promoters and variants are well known in the art and include, but are not limited to, PLteto1, galP1, PLlacO1, Pfnrs. In some embodiments, the nucleotide sequences of exemplary inducible promoters comprise a nucleotide sequence selected from the group consisting of SEQ ID NOs: 55-58 as shown in Table 2, and homologous sequences thereof having at least 80% (e.g. at least 85%, 90%, 91%, 92%, 93%, 94%, 95%, 96%, 97%, 98%, 99%) sequence identity. In some embodiments, the inducible promoter comprises the nucleotide sequence of SEQ ID NO: 58.

In some embodiments, the promoter is an endogenous promoter, or an exogenous promoter. An “exogenous promoter” as used herein refers to a promoter in operable combination with a coding region wherein the promoter is not the promoter naturally associated with the coding region in the genome of an organism. The promoter which is naturally associated or linked to a coding region in the genome is referred to as the “endogenous promoter” for that coding region.

The term “ribosomal binding site” (“RBS”) as used herein refers to a sequence on mRNA that the ribosome binds to when initiating protein translation. The RBS is approximately 35 nucleotides long and contains three discrete domains: (1) the Shine-Dalgarno (SD) sequence, (2) a spacer region, and (3) the first five to six codons of the Coding Sequence (CDS). RBSs and variants are well known in the art and include, but are not limited to, USP45, Synthesized, Amuc_1102, OmpA. The nucleotide sequences of exemplary RBSs comprise a nucleotide sequence selected from the group consisting of SEQ ID NOs: 70-73 as shown in Table 2, and homologous sequences thereof having at least 80% (e.g. at least 85%, 90%, 91%, 92%, 93%, 94%, 95%, 96%, 97%, 98%, 99%) sequence identity.

The term “cistron” as used herein refers to a segment of nucleic acid sequence that is transcribed and that codes for a polypeptide. Cistrons and variants are well known in the art and include, but are not limited to, GFP, BCD2, luciferase, MBP. The nucleotide sequences of exemplary cistrons comprise a nucleotide sequence selected from the group consisting of SEQ ID NOs: 66-69 as shown in Table 2, and homologous sequences thereof having at least 80% (e.g. at least 85%, 90%, 91%, 92%, 93%, 94%, 95%, 96%, 97%, 98%, 99%) sequence identity.

The term “terminator” as used herein refers to an enzymatically incorporable nucleotide which prevents subsequent incorporation of nucleotides to the resulting polynucleotide chain and thereby halts polymerase-mediated extension. In some embodiments, the terminator used in the present disclosure is T7 terminator. In some embodiments, the terminator comprises a nucleotide sequence selected from the group consisting of SEQ ID NOs: 74-75 as shown in Table 2, and homologous sequences thereof having at least 80% (e.g. at least 85%, 90%, 91%, 92%, 93%, 94%, 95%, 96%, 97%, 98%, 99%) sequence identity.

In certain embodiments, in the exogenous expression cassette provided herein, the polynucleotide sequence that encodes Amuc_1100 is operably linked to a signal peptide.

The term “signal peptide” or “signal sequence” as used herein refers to a peptide that can be used to secrete the heterologous polypeptide into the periplasm or medium of the cultured bacteria or to secrete the Amuc_1100 polypeptide into the periplasm. The signals for the heterologous polypeptide may be homologous to the bacteria, or they may be heterologous, including signals native to the polypeptide being produced in the bacteria. For Amuc_1100, the signal sequence is typically that which is endogenous to the bacterial cells, although it need not be as long as it is effective for its purpose. Non-limiting examples of the secretion peptides include OppA (ECOLIN_07295), OmpA, OmpF, cvaC, TorA, fdnG, dmsA, PelB, HlyA, Adhesin (ECOLIN_19880), DsbA (ECOLIN_21525), Gltl (ECOLIN_03430), GspD (ECOLIN_16495), HdeB (ECOLIN_19410), MalE (ECOLIN_22540), PhoA (ECOLIN_02255), PpiA (ECOLIN_18620), To1B, tort, mglB, and lamB.

In certain embodiments, the signal peptide provided herein for Amuc_1100 secretion comprises Usp45, OmpA, DsbA, pelB, celCD, sat, or the endogenous signal peptide of Amuc_1100. The nucleotide sequences encoding exemplary signal peptides comprise a nucleotide sequence selected from the group consisting of SEQ ID NOs: 59-65 as shown in Table 2, and homologous sequences thereof having at least 80% (e.g. at least 85%, 90%, 91%, 92%, 93%, 94%, 95%, 96%, 97%, 98%, 99%) sequence identity. In some embodiments, the signal peptide comprises an amino acid sequence selected from the group consisting of SEQ ID NOs: 76-82 as shown in Table 3, and homologous sequences thereof having at least 80% (e.g. at least 85%, 90%, 91%, 92%, 93%, 94%, 95%, 96%, 97%, 98%, 99%) sequence identity. In some embodiments, the signal peptide operably linked at the N-terminus of the Amuc_1100.

In some embodiments, the expression cassette comprises a nucleotide sequence that encodes Amuc_100 operably linked to a signal peptide and an autotransporter domain, wherein the signal peptide and the autotransporter domain are operably linked at the opposite terminus of the Amuc_1100, e.g. the signal peptide operably linked at the N-terminus of the Amunc_1100, and the autotransporter domain operably linked at the C-terminus of the Amunc_1100. Such construct can be useful in Type V auto-secretion-mediated secretion, where the N-terminal signal peptide is removed upon translocation of the precursor protein from the cytoplasm into the periplasmic compartment by a native secretion system such as Sec system, and further, once the auto-secretor is translocated across the outer membrane, the C-terminal autotransporter domain can be removed by either an autocatalytic or protease-catalyzed e.g., OmpT cleavage thereby releasing the mature protein (e.g., Amuc_1100 polypeptide) into the extracellular milieu.

TABLE 2
The nucleotide sequences of exemplary promoters, RBS, cistron, signal peptides,
and terminators

| Regulatory | | | SEQ |
|---|---|---|---|
| element | Name | Nucleotide Sequence | ID NO. |
| Constitutive | BBa_J23119 | ttgacagctagctcagtcctaggtataatgctagc | 14 |
| promoter | BBa_J23101 | tttacagctagctcagtcctaggtattatgctagc | 15 |
| | BBa_J23102 | ttgacagctagctcagtcctaggtactgtgctagc | 16 |
| | BBa_J23103 | ctgatagctagctcagtcctagggattatgctagc | 17 |
| | BBa_J23109 | tttacagctagctcagtcctagggactgtgctagc | 18 |
| | BBa_J23110 | tttacggctagctcagtcctaggtacaatgctagc | 19 |
| | BBa_J23114 | tttatggctagctcagtcctaggtacaatgctagc | 20 |
| | BBa_J23117 | ttgacagctagctcagtcctagggattgtgctagc | 21 |
| | USP45_ | aaagtgttttgtaatcataaagaaatattaaggtggg | 22 |
| | promoter | gtaggaatagtataatatgtttattcaaccgaacttaa | |
| | | tg | |
| | Amuc_1102_ | gggccgagcatgtggaaacacctctgccaggtca | 23 |
| | promoter | agcccgcagccatgccgcatgtttttttcttgaccgc | |
| | | ccgggcagaaccgtcgggcggagccgtgtcatg | |
| | | ctccacacacgctacatggaaagaaacaagagatt | |
| | | tttgctagccacagttcggtttttggtgtaactaatc | |
| | OmpA_ | gtaaatttaggattaatcctggaactttttttgtcgcc | 24 |
| | promoter | cagccaatgctttcagtcgtgactaattttccttgcg | |
| | | gaggcttgtctgaagcggtttccgcgattttcttctgt | |
| | | aaattgtcgctgacaaaaaagattaaacgtaccttat | |
| | | acaagacttttttttcatatgcctgacggagttcacac | |
| | | ttgtaagttttcaactacgttgtagactttacategcc | |
| | | aggggtgctcggcataagccgaagatatcggtag | |
| | | agttaatattgagcagatccccggtgaaggatttaa | |
| | | ccgtgttatctcgttggagatattcatggcgtattttg | |
| | | gatga | |
| | BBa_J23100 | ttgacggctagctcagtcctaggtacagtgctagc | 25 |
| | BBa_J23104 | ttgacagctagctcagtcctaggtattgtgctagc | 26 |
| | BBa_J23105 | tttacggctagctcagtcctaggtactatgctagc | 27 |
| | BBa_I14018 | gtttatacataggcgagtactctgttatgg | 28 |
| | BBa_J45992 | ggtttcaaaattgtgatctatatttaacaa | 29 |
| | BBa_J23118 | ttgacggctagctcagtcctaggtattgtgctagc | 30 |
| | BBa_J23116 | ttgacagctagctcagtcctagggactatgctagc | 31 |
| | BBa_J23115 | tttatagctagctcagcccttggtacaatgctagc | 32 |
| | BBa_J23113 | ctgatggctagctcagtcctagggattatgctagc | 33 |
| | BBa_J23112 | ctgatagctagctcagtcctagggattatgctagc | 34 |
| | BBa_J23111 | ttgacggctagctcagtcctaggtatagtgctagc | 35 |
| | BBa_J23108 | ctgacagctagctcagtcctaggtataatgctagc | 36 |
| | BBa_J23107 | tttacggctagctcagccctaggtattatgctagc | 37 |
| | BBa_J23106 | tttacggctagctcagtcctaggtatagtgctagc | 38 |
| | BBa_I14033 | agaggttccaactttcaccataatgaaaca | 39 |
| | BBa_K256002 | caccttcggggggcctttctgcgtttata | 40 |
| | BBa_K1330002 | ggctagctcagtcctaggtactatgctagc | 41 |
| | BBa_J44002 | aaagtgtgacgccgtgcaaataatcaatgt | 42 |
| | BBa_J23150 | ggctagctcagtcctaggtattatgctagc | 43 |
| | BBa_I14034 | taaacaactaacggacaattctacctaaca | 44 |
| | Oxb19 | aagctgttgtgaccgcttgctctagccagctatcga | 45 |
| | | gttgtgaaccgatccatctagcaattggtctcgatct | |
| | | agcgataggcttcgatctagctatgtatcactcatta | |
| | | ggcaccccaggctttacactttatgcttccggctcgt | |
| | | ataatgtgtggtgctggttagcgcttgctat | |
| | oxb20 | aagctgttgtgaccgcttgctctagccagctatcga | 46 |
| | | gttgtgaaccgatccatctagcaattggtctcgatct | |
| | | agcgataggcttcgatctagctatgtagaaacgcc | |
| | | gtgtgctcgatcgcttgataaggtccacgtagctgc | |
| | | tataattgcttcaacagaacatattgactatccggtat | |
| | | tacccggc | |
| | BBa_K088007 | catacgccgttatacgttgtttacgctttg | 47 |
| | Ptet | taattcctaatttttgttgacactctatcgttgatagag | 48 |
| | | ttattttaccactccctatcagtgatagagaaaa | |
| | Ptrc | ttgacaattaatcatccggctcgtataatgtgtggaa | 49 |
| | | ttgtgag | |
| | PlacUV5 | cccaggctttacactttatgcttccggctcgtataat | 50 |
| | | gtgtggaattgtgag | |
| | BBa_K292001 | tgctagctactagagattaaagaggagaaa | 51 |
| | BBa_K292000 | ggctagctcagtcctaggtacagtgctagc | 52 |
| | BBa_K137031 | ccccgaaagcttaagaatataattgtaagc | 53 |
| | BBa_K137029 | atatatatatatatataatggaagcgtttt | 54 |
| Inducible | PLteto1 | tccctatcagtgatagagattgacatccctatcagtg | 55 |
| promoter | | atagagatactgagcacatcagcaggacgcactg | |
| | | acc | |
| | galP1 | attccactaatttattccatgtcacacttttcgcatcttt | 56 |
| | | gttatgctatggttatttcataccataa | |
| | PLlacO1 | ataaatgtgagcggataacaattgacattgtgagc | 57 |
| | | ggataacaagatactgagcacatcagcaggacgc | |
| | | actgacc | |
| | Pfnrs | aaaaacgccgcaaagtttgagcgaagtcaataaa | 58 |
| | | ctctctacccattcagggcaatatctctctt | |
| Signal | Usp45 | atgaagaaaaagatcattagcgcgatcctgatgag | 59 |
| peptide | | caccgtgattctgagcgcggcggcgccgctgagc | |
| | | ggtgtttatgcg | |
| | OmpA | atgaagaaaaccgcgattgcgattgcggtggcgc | 60 |
| | | tggcgggtttcgcgaccgttgcgcaggcg | |
| | DsbA | atgaagaaaatctggctggcgctggcgggtctggt | 61 |
| | | gctggcgttcagcgcgagcgcg | |
| | pelB | atgaagtacctgctgccgaccgcggcggcgggtc | 62 |
| | | tgctgctgctggcggcgcagccggcgatggcg | |
| | celCD | atggaaggaaacactcgtgaagacaattttaaacat | 63 |
| | | ttattaggtaatgacaatgttaaacgc | |
| | sat | atgaataaaatatactcccttaaatatagtgctgcca | 64 |
| | | ctggcggactcattgctgtttctgaattagcgaaaa | |
| | | gagtttctggtaaaacaaaccgaaaacttgtagcaa | |
| | | caatgttgtctctggctgttgccggtacagtaaatgc | |
| | | a | |
| | Endogenous | atgagcaactggatcaccgacaacaaaccggctg | 65 |
| | signal peptide | cgatggttgcgggtgtgggcctgctgctgttcctg | |
| | of Amuc_1100 | ggtctgagcgcgaccggctac | |
| Cistron | GFP | atgcgtaaaggcgaagagaaggaggttaactga | 66 |
| | BCD2 | atgaaagcaattttcgtactgaaacatcttaatcatg | 67 |
| | | ctaaggaggttttctaa | |
| | luciferase | atgattatgtccggttataaggaggttaactga | 68 |
| | MBP | atgaaaatcgaagcaggtaaactggtacagaagg | 69 |
| | | aggttaactga | |
| RBS | USP45 | ggaggaaaaattaaaaaagaac | 70 |
| | Synthesized | aaagaggagaaa | 71 |
| | Amuc_1102 | agggaa | 72 |
| | OmpA | taacgagg | 73 |
| Terminator | Terminator1 | cgagctcgatagtgctagtgtagatcgctactaga | 74 |
| | | gccaggcatcaaataaaacgaaaggctcagtcga | |
| | | aagactgggcctttcgttttatctgttgtttgtcggtg | |
| | | aacgctctctactagagtcacactggctcaccttcg | |
| | | ggtgggcctttctgcgtttatatactagaagcggcc | |
| | | gctgcag | |
| | Terminator2 | cgagctcgatagtgctagtgtagatcgctactaga | 75 |
| | | gccaggcatcaaataaaacgaaagactgggccttt | |
| | | cgttttatctgttgtttgtcggtgaacgctctctactag | |
| | | agtcacactggctcaccttcggggggcctttctgc | |
| | | gtttatatactagaagcggccgctgcag | |

TABLE 3
Amino acid sequence of exemplary signal peptides

| SEQ ID NO. | Name | Amino Acid Sequence |
|---|---|---|
| 76 | Usp45 | MKKKIISAILMSTVILSAAAPLSGVYA |
| 77 | OmpA | MKKTAIAIAVALAGFATVAQA |
| 78 | DsbA | MKKIWLALAGLVLAFSASA |
| 79 | pelB | MKYLLPTAAAGLLLLAAQPAMA |
| 80 | celCD | MEGNTREDNFKHLLGNDNVKR |
| 81 | sat | MNKIYSLKYSAATGGLIAVSELAKRVSG |
| | | KTNRKLVATMLSLAVAGTVNA |
| 82 | Endogenous | MSNWITDNKPAAMVAGVGLLLFLGLSA |
| | signal peptide of | TGY |
| | Amuc_1100 | |

ii. Secretion System and/or Export Pathway in the Host Cell

In some embodiments, the signal peptide can be processed by an export pathway and/or a secretion system present in the genetically engineered host cell (e.g. genetically engineered bacteria). Signal peptide is normally at N-terminus of the exported precursor protein, and can direct the precursor protein to the export pathway in the bacterial cytoplasmic membrane. A secretion system is able to remove the signal peptide from the precursor protein before secreting the mature protein from the engineered host cell such as bacteria.

As used herein, the term “secretion system” is used interchangeably herein with “export system” and “export pathway”, refers to a native or non-native secretion mechanism capable of secreting or exporting an expressed polypeptide product (e.g. Amuc_1100 polypeptide) from the microbial, e.g., bacterial cytoplasm. From outside inward in a Gram-negative bacteria such as E. coli, there are outer membrane (OM), peptidoglycan cell wall, periplasm, inner membrane (IM), and cytoplasm compartments. The OM is a very effective and selective permeability barrier. The OM is a lipid bilayer consisting of phospholipids at the inner leaflet and glycolipids at the outer leaflet as well as lipoproteins and beta-barrel proteins. The OM is stapled to the underlying peptidoglycan by a lipoprotein called Lpp. The periplasm is densely packed with proteins and it is more viscous than the cytoplasm. The IM is a phospholipid bilayer and the major place holding membrane proteins that function in energy production, lipid biosynthesis, protein secretion, and transport.

In bacteria, there are two common protein export pathways including the ubiquitous general secretion (or Sec) pathway and the twin-arginine translocation (or Tat) pathway. Usually, the Sec pathway handles higher molecular weight precursor proteins in an unfolded state with a signal peptide targeting the substrate proteins to the membrane-bound Sec translocase. The precursor target proteins are delivered to the translocase and passed through the SecYEG pore by SecA, SecD and SecF. Chaperones SecB, GroEL-GroES, DnaK-DnaJ-GrpE are assisting the target proteins transport. The Tat pathway normally transports proteins in a completely folded or even oligomeric state and consists of components TatA, TatB and TatC in both Gram-negative and—positive bacteria. After the membrane translocation, the signal peptide is removed by signal peptidase and the mature protein is secreted.

For Gram-negative bacteria, they have specialized one-step secretion system, and non-limiting examples of secretion systems include Sat secretion system, type I secretion system (TlSS), type II secretion system (T2SS), type III secretion system (T3SS), type IV secretion system (T4SS), type V secretion system (T5SS), type VI secretion system (T6SS), and resistance-nodulation-division (RND) family of multi-drug efflux pumps, various single membrane secretion systems.

In some embodiments, the secretion system is native or non-native to the genetically engineered host cell. “Native” to the host cell means the secretion system is normally present in the host cell, while “non-native” to the host cell means the secretion system is not normally present in the host cell, e.g., an extra secretion system, such as a secretion system from a different species, strain, or substrain of bacteria or virus, or a secretion system that is modified and/or mutated as compared to the unmodified secretion system from the host cell of the same subtype.

In some embodiments, the secretion system is engineered and/or optimized such that at least one outer membrane protein encoding gene in the genetically engineered host cell is deleted, inactivated, or suppressed. In some embodiments, such outer membrane protein is selected from the group consisting of OmpC, OmpA, OmpF, OmpT, pldA, pagP, tolA, Pal, To1B, degS, mrcA and 1pp.

In order to make Gram-negative bacteria (for example EcN) capable of secreting desired therapeutic protein or peptide, it is needed to genetically engineer bacteria host and make them have a “leaky” or destabilized outer membrane, yet not to impact host bacteria chemical physical activity. Genes for example 1pp, ompC, ompA, ompF, ompT,pldA, pagP, tolA, tolB, pal, degS, degP, and nlpI can be deleted or mutagenized to make “leaky” outer membrane. Lpp is the most abundant polypeptide in the bacterial cell and functions as the primary ‘staple’ of the bacterial cell wall to the peptidoglycan. Deletion of 1pp had minimal impact on bacterial growth but increased the secretion of Amuc_1100, for example by at least one fold. An inducible promoter can be used to replace the selected gene(s)' endogenous promoter to minimize the negative impact on cell viability.

The “deletion” or “inactivation” or “suppression” of a gene or coding region means causing the enzyme or protein encoded by that gene or coding region not to be produced, or to be produced in a host cell in an inactive form, or to be produced in a host cell at a level that is lower than the level found in the wild type version of the host cell under the same or similar growth conditions. This can be accomplished by, for example, one or more of the following methods: (1) homologous recombination, (2) RNA interference based techniques, (3) ZFN and TALEN, (4) CRISPR/Cas systems.

In some embodiments, the secretion system is engineered such that at least one Chaperone protein encoding gene is amplified, overexpressed or activated.

Chaperone proteins are involved in many important biological processes such as protein folding and aggregation of oligomeric protein complexes, maintaining protein precursors in an unfolded state to facilitate protein transmembrane transport, and enabling denatured proteins to be disaggregated and repaired. It is mainly to assist other peptides to maintain the normal conformation to form the correct oligomeric structure, thereby exerting normal physiological functions. Various Chaperon proteins are well-known in the art. In some embodiments, the Chaperone protein is selected from the group consisting of. dsbA, dsbC, dnaK, dnaJ, grpE, groES, groEL, tig, fkpA, surA, skp, PpiD and DegP. In some embodiments, the Chaperone protein is selected from the group consisting of Ssalp, Ssa2p, Ssa3p and Ssa4p from the cytosolic SSA subfamily of 70 kDa heat shock proteins (Hsp70), BiP, Kar2, Lhsl, Sill, Sec63, Protein disulfide isomerase Pdilp.

The “overexpressed” or “overexpression” of a gene or coding region means causing the enzyme or protein encoded by that gene or coding region to be produced in a host cell at a level that is higher than the level found in the wild type version of the host cell under the same or similar growth conditions. This can be accomplished by, for example, one or more of the following methods: (1) installing a stronger promoter, (2) installing a stronger ribosome binding site, such as a DNA sequence of 5′-AGGAGG, situated about four to ten bases upstream of the translation start codon, (3) installing a terminator or a stronger terminator, (4) improving the choice of codons at one or more sites in the coding region, (5) improving the mRNA stability, and (6) increasing the copy number of the gene, either by introducing multiple copies in the chromosome or placing the cassette on a multicopy plasmid. An enzyme or protein produced from a gene that is overexpressed is said to be “overproduced”. A gene that is being “overexpressed” or a protein that is being “overproduced” can be one that is native to a host cell, or it can be one that has been transplanted by genetic engineering methods from a different organism into a host cell, in which case the enzyme or protein and the gene or coding region that encodes the enzyme or protein is called “foreign” or “heterologous”. Foreign or heterologous genes and proteins are overexpressed and overproduced, since they are not present in the unengineered host cell.

For yeast, the secretion system primarily includes nascent protein translocation, protein folding in the endoplasmic reticulum (ER), glycosylation, protein sorting, and trafficking. Newly synthesized membrane or secretory proteins are recognized by a signal recognition particle (SRP) as soon as the signal peptide emerges from the ribosome during translation, then targeted to post-translational secretion through the Sec61 or Sshl translocon pore. Various approaches have been implemented to improve heterologous protein production in yeast: (1) engineering the signal peptide and increasing the promoter strength and plasmid copy numbers; (2) engineering the post-translational pathway of host strains, for example, over-expression of ER folding chaperones and vesicle trafficking components and decreasing intercellular and extracellular proteolysis. Exemplary yeast secretory signal peptides for secretion of heterogenous proteins include Saccharomyces cerevisiae α-mating factor (α-MF) pre pro-peptide, signal peptide of inulinase from Kluyveromyces marxianus, signal peptide of PHAE from Phaseolus vulgaris agglutinin, signal peptide of viral prepro toxin, signal peptide of Rhizopus oryzae amylase or of hydrophobin signal sequence from fungal Trichoderma reesei.

iii. Genome Integration Site

In some embodiments, the exogenous expression cassette is integrated into the genome of the genetically engineered host cell. In some embodiments, the exogenous expression cassette is integrated into the genome of the genetically engineered host cell by CRISPR-Cas genome editing system. Any suitable host cells provided herein can be engineered such that the exogenous expression cassette is integrated into the genome.

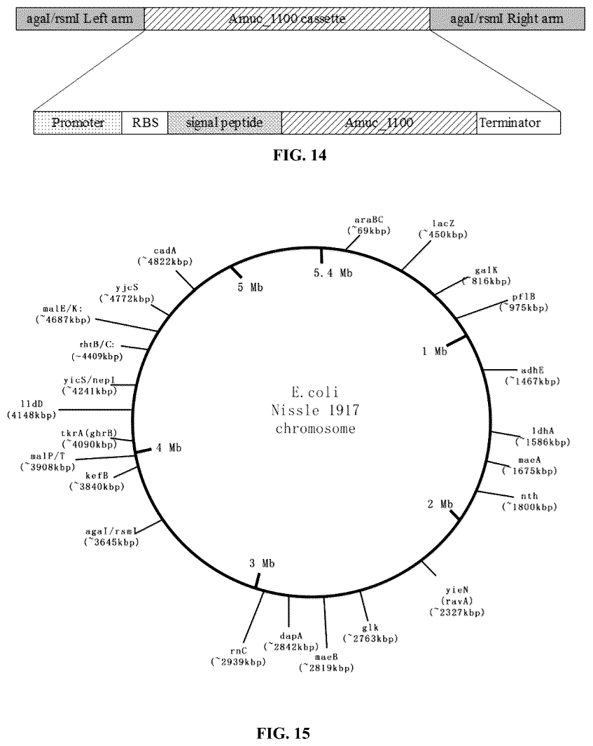

In some embodiments, the genetically engineered host cell is Escherichia coli strain Nissle 1917 (EcN), and the exogenous expression cassette is integrated into a site in EcN genome. Suitable integration sites in the EcN genome can be any site listed in Table 4 (shown in FIG. 14, agaI/rsmI is shown as an example). In some embodiments, the suitable integration sites in the EcN genome are integration sites agaI/rsmI.

The genome sites at EcN genome for Amuc_1100 integration in this invention include any site listed in Table 4. Without wishing to be bound by any theory, but it is believed that the genome sites of EcN listed in Table 4 are advantageous in at least one of the following characteristics for insertion of the expression cassette for Amuc_1100: (1) the bacterial gene(s) impacted by the site's engineering are not essential for EcN's growth and do not change the host bacteria biochemical and physiological activity, (2) the site can be easily edited, and (3) the Amuc_1100 gene cassette in the site can be transcribed. The map of the integration sites are shown in FIG. 15. The sgRNA sequences used to edit corresponding genome sites in EcN are shown in Table 4 below.

TABLE 4
sgRNA sequence used to edit corresponding genome site in EcN.

| Integration site | sgRNA | SEQ ID NO. |
|---|---|---|
| agaI/rsmI (~3645 kbp) | ggcgagttaacgacgacaca | 83 |
| araBC (~69 kbp) | cgttgaactgggtgtggaat | 84 |
| cadA (~4822 kbp) | ctgaaaccgctgcggcgatg | 85 |
| cadA (~4822 kbp) | gttcgcagtggaagtaccgt | 86 |
| dapA (~2842 kbp) | ctgtgcaaacaagtgtctca | 87 |
| kefB (~3840 kbp) | gccggaagacactatgaagc | 88 |
| lacZ (~450 kbp) | tcgcacagcgtgtaccacag | 89 |
| maeB (~2819 kbp) | gaaggggaagaggcgcgcgt | 90 |
| malE/K: (~4687 kbp) | cggtttagttcacagaagcc | 91 |
| malP/T (~3908 kbp) | ttgcgtattttcaaaaagcg | 92 |
| rhtB/C: (~4409 kbp) | tcatcagagtaagtcggata | 93 |
| yicS/nepI (~4241 kbp) | ctgaccaacgcttctttacc | 94 |
| adhE (~1467 kbp) | ccgaagtccctgtgtgcttt | 95 |
| galK (~816 kbp) | ccctgccactcacaccattc | 96 |
| glk (~2763 kbp) | ccttctcctggcaagcttac | 97 |
| ldhA (~1586 kbp) | cgacaagaagtacctgcaac | 98 |
| lldD (~4148 kbp) | gttatcgtgatgcgcattct | 99 |
| maeA (~1675 kbp) | taacacccagcccgatgccc | 100 |
| nth (~1800 kbp) | atattactggaacaacataa | 101 |
| pflB (~975 kbp) | ccgtgacgttatccgcacca | 102 |
| rnC (~2939 kbp) | tatcgccttcatccacacga | 103 |
| tkrA (ghrB) (~4090 kbp) | ccgggctggatgtcttcgaa | 104 |
| yieN (ravA) (~2327 kbp) | gagggtgagccataatgaag | 105 |
| yjcS (~4772 kbp) | ggatatgtggggtaacgacg | 106 |
| LPP (~1846 kbp) | acgttcaggctgctaaagat | 107 |

iv. Auxotroph

In some embodiments, the host cell of the present disclosure further comprises at least one inactivation or deletion in an auxotroph-related gene.

In order to generate an environment-friendly bacteria, some essential genes that necessary for bacterial cell survival can be deleted or inactivated by mutagenesis, making the engineered bacteria become auxotroph. The term “auxotroph” as used herein refers to a host cell (e.g. a strain of microorganism) requiring for growth an external source of a specific metabolite that cannot be synthesized because of an acquired genetic defect. The term “auxotroph-related gene” as used herein refers to a gene required for the host cell (e.g. microorganism such as bacteria) to survive. The auxotroph-related gene can be necessary for the microorganism to produce for a nutrient essential for survival or growth, or can be required for detection of a signal in an environment that modulates activity of the transcription factor, wherein absence of the signal would lead to the cell death.

In some embodiments, an auxotrophic modification is intended to cause the microorganism to die in the absence of an exogenously added nutrient essential for survival or growth because they lack the gene(s) necessary to produce that essential nutrient. In some embodiments, any of the genetically engineered bacteria described herein also comprise a deletion or mutation in a gene required for cell survival and/or growth.

Various auxotroph-related genes in bacteria are well-known in the art. Exemplary auxotroph-related genes include, but not limited to, thyA, cysE, glnA, ilvD, leuB, lysA, serA, metA, glyA, hisB, ilvA, pheA, proA, thrC, trpC, tyrA, uraA, dapF, flhD, metB, metC, proAB, yhbV, yagG, hemB, secD, secF, ribD, ribE, thiL, dxs, ispA, dnaX, adk, hemH, IpxH, cysS, fold, rp1T, infC, thrS, nadE, gapA, yeaZ, aspS, argS, pgsA, yeflA, metG, folE, yejM, gyrA, nrdA, nrdB, folC, accD, fabB, gltX, ligA, zipA, dapE, dapA, der, hisS, ispG, suhB, tadA, acpS, era, rnc, fisB, eno, pyrG, chpR, Igt, ft>aA, pgk, yqgD, metK, yqgF, plsC, ygiT, pare, ribB, cca, ygjD, tdcF, yraL, yihA, ftsN, murl, murB, birA, secE, nusG, rp1J, rp1L, rpoB, rpoC, ubiA, plsB, lexA, dnaB, ssb, alsK, groS, psd, orn, yjeE, rpsR, chpS, ppa, valS, yjgP, yjgQ, dnaC, ribF, IspA, ispH, dapB, folA, imp, yabQ, flsL, fisl, murE, murF, mraY, murD, ftsW, murG, murC, ftsQ, ftsA, ftsZ, IpxC, secM, secA, can, folK, hemL, yadR, dapD, map, rpsB, in/B,nusA, ftsH, obgE, rpmA, rplU, ispB, murA, yrbB, yrbK, yhbN, rpsl, rplM, degS, mreD, mreC, mreB, accB, accC, yrdC, def, fint, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yr/F, asd, rpoH, ftsX, ftsE, ftsY, frr, dxr, ispU, rfaK, kdtA, coaD, rpmB, djp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, IpxA, IpxB, dnaE, accA, tilS, proS, yafF, tsf, pyrH, olA, rlpB, leuS, Int, glnS, fidA, cydA, in/A, cydC, ftsK, lolA, serS, rpsA, msbA, IpxK, kdsB, mukF, mukE, mukB, asnS, fabA, mviN, rne, yceQ, fabD, fabG, acpP, tmk, holB, lo1C, loiD, lolE, purB, ymflC, minE, mind, pth, rsA, ispE, l1iB, hemA, prfA, prmC, kdsA, topA, ribA, fabi, racR, dicA, yd B, tyrS, ribC, ydiL, pheT, pheS, yhhQ, bcsB, glyQ, yibJ, and gpsA.

In one modification, the essential gene thyA is deleted or replaced by another gene making the genetically engineered bacteria dependent on exogenous thymine to grow or survive. Adding thymine to growth media or the human gut naturally having high thymine level can support the growth and survival of thyA auxotroph bacteria. This kind of modification is to ensure that the genetically engineered bacteria cannot grow and survive outside of the gut or in the environment that in lack of the auxotrophic gene product.

In some embodiments, the host cell is an auxotroph for one or more substances selected from the group consisting of uracil, leucine, histidine, tryptophan, lysine, methionine, adenine, and non-naturally occurring amino acid. In some embodiments, the non-naturally occurring amino acid is selected from the group consisting of 1-4,4′-biphenylalanine, p-acetyl-1-phenylalanine, p-iodo-1-phenylalanine, and p-azido-1-phenylalanine.

In some embodiments, the host cell comprises an allosterically regulated transcription factor which is capable of detecting a signal in an environment that modulates activity of the transcription factor, wherein absence of the signal would lead to the cell death. Such “signaling molecule-transcription factor” pairs may include any one or more selected from the group consisting of tryptophan-TrpR, IPTG-LacI, benzoate derivatives-XylS, ATc-TetR, galactose-GalR, estradiol-estrogen receptor hybrid protein, cellobiose-CelR, and homoserine lactone-luxR.

v. Deletion of Endogenous Plasmid

In some embodiments, the genetically engineered bacteria has deletion of one or more endogenous plasmids.

Some chassis bacteria host comprise endogenous plasmid(s) which consume significant resources for their transcription. Without being bound to any theory, it is believed that it is useful to delete these endogenous plasmids to release sources that better used for heterologous gene expression.

The endogenous plasmid(s) can be removed from a target host cell by methods known in the art. For example, EcN comprises two endogenous plasmids pMUT1 and pMUT2, which can be removed from EcN by the methods below. pCBT003_pMUT1_sgRNA expressing sgRNA of pMUT1 and pCBT001 plasmids are transformed into EcN to remove pMUT1 (EcN A pMUT1). To delete pMUT2, a kanamycin-resistant plasmid pMUT2 (pMUT2-kana) is mimicked, made, and transferred into EcN A pMUT1. Then, the transformed strain are selected on LB agar plate supplemented with kanamycin. EcN A pMUT1/pMUT2-kana is grew in LB liquid supplemented with increased concentration of kanamycin for several generation until pMUT2 was completely replaced by pMUT2-kana. The pMUT2 replaced clone of EcN A pMUT1/pMUT2-kana is transformed with pCBT001, and then the plasmid pCBT003_Kana-sgRNA which expressing sgRNA of kanamycin gene to cut and remove pMUT2-kana. The EcN strain that depleting two endogenous plasmids can be verified by PCR detection.

vi. Combination of regulatory elements and integration sites for enhanced secretion yield

The secretion yield of heterogenous protein in a genetically engineered host cell such as bacteria is usually low. In order to treat a disease, the host cell must be able to produce and secrete a sufficient amount of Amuc 1100, and should also maintain health of the host cell. The present inventors tested a variety of genome integration sites, regulatory elements, and secretion systems, and identified combinations that can provide for unexpected effects in Amuc_1100 expression and secretion.

In certain embodiments, the genetically engineered host cell (e.g. bacteria) are characterized in: (1) having a combination of regulatory elements that can increase Amuc_1100 transcription; (2) having more than one copies of Amuc_1100 gene integrated into multiple genome sites; (3) having modified signal peptide for better protein secretion, (4) having a modified secretion system that generates a “leaky” outer membrane; (5) having additional chaperon genes; (6) having reduced or removed endogenous plasmids, or any combination thereof.

The genome integration site, promoter, RBS and cistron have been intensively tested individually or in combination by the inventors, to increase Amuc_1100 gene transcription.

In certain embodiments, the genetically engineered host cell comprises a combination of promoter, RBS, and cistron, that provides for increased transcription of Amuc_1100 gene in the genetically engineered host cell. The promoter, RBS, and cistron can be selected from the list in Table 2, and any of the combinations are encompassed herein.

In some embodiments, the promoter is selected from the group consisting of BBa_J23119, BBa_J23101, BBa_J23102, BBa_J23103, BBa_J23109, BBa_J23110, BBa_J23114, BBa_J23117, USP45_promoter, Amuc_1102_promoter, OmpA_promoter, BBa_J23100, BBa_J23104, BBa_J23105, BBa 114018, BBa_J45992, BBa_J23118, BBa_J23116, BBa_J23115, BBa_J23113, BBa_J23112, BBa_J23111, BBa_J23108, BBa_J23107, BBa_J23106, BBa_114033, BBa_K256002, BBa_K1330002, BBa_J44002, BBa_J23150, BBa_114034, Oxbl9, oxb20, BBa_K088007, Ptet, Ptrc, PlacUV5, BBa_K292001, BBa_K292000, BBa_K137031, BBa_K137029, PLtetol, galP1, PLlacO1, and Pfnrs. In some embodiments, the promoter comprises BBa_J23101. In some embodiments, the promoter is BBa_J23101. In some embodiments, the RBS is selected from the group consisting of USP45, Synthesized, Amuc_1102, and OmpA. In some embodiments, the cistron is selected from the group consisting of GFP, BCD2, luciferase, and MBP.

In some embodiments, the promoter comprises BBa_J23101, the RBS comprises at least one (e.g. 1, 2, 3, 4) selected from the group consisting of USP45, Synthesized, Amuc_1102, and OmpA, and/or the cistron comprises at least one (e.g. 1, 2, 3, 4) selected from the group consisting of GFP, BCD2, luciferase, and MBP.

In some embodiments, the promoter comprises Pfnrs, the RBS comprises at least one (e.g. 1, 2, 3, 4) selected from the group consisting of USP45, Synthesized, Amuc_1102, and OmpA, and/or the cistron comprises at least one (e.g. 1, 2, 3, 4) selected from the group consisting of GFP, BCD2, luciferase, and MBP.

In any of the above provided embodiments, the terminator is selected from any terminator listed in Table 2. In some embodiments, the terminator is T7 terminator.

In some embodiments, the promoter comprises BBa_J23101 or Pfnrs, the RBS comprises SEQ ID NO: 71, the signal peptide comprises USP45, and the terminator comprises Terminator1. In some embodiments, the promoter comprises BBa_J23101 or Pfnrs, the RBS comprises SEQ ID NO: 71, the signal peptide comprises USP45, and the terminator comprises Terminator2. In some embodiments, the promoter comprises SEQ ID NO: 15, the RBS comprises SEQ ID NO: 71, the nucleotide sequence of the signal peptide comprises SEQ ID NO: 59, and the terminator comprises SEQ ID NO: 74. In some embodiments, the promoter comprises SEQ ID NO: 15, the RBS comprises SEQ ID NO: 71, the nucleotide sequence of the signal peptide comprises SEQ ID NO: 59, and the terminator comprises SEQ ID NO: 75.

Exemplary optimized Amuc_100 gene cassettes are shown in SEQ ID NO: 108 and SEQ ID NO: 148 below.

SEQ ID NO: 108
(Promoter J23101 RBS usp45 his tag Enterokinase
Amuc_1100_terminator1):

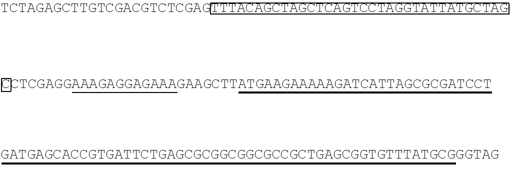

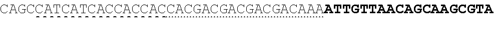

GCGAGCTGGATAAGAAAATCAGCATTGCGGCGAAAGAGATCAAGAGCGCGAAC
GCGGCGGAAATTACCCCGAGCCGTAGCAGCAACGAGGAACTGGAGAAAGAACT
GAACCGTTACGCGAAGGCGGTTGGTAGCCTGGAAACCGCGTATAAACCGTTTCT
GGCGAGCAGCGCGCTGGTGCCGACCACCCCGACCGCGTTCCAAAACGAGCTGA
AAACCTTTCGTGACAGCCTGATCAGCAGCTGCAAGAAAAAGAACATCCTGATTA
CCGATACCAGCAGCTGGCTGGGCTTCCAGGTTTACAGCACCCAAGCGCCGAGC
GTGCAGGCGGCGAGCACCCTGGGTTTTGAGCTGAAAGCGATTAACAGCCTGGT
TAACAAGCTGGCGGAATGCGGCCTGAGCAAATTCATCAAGGTGTATCGTCCGCA
GCTGCCGATTGAAACCCCGGCGAACAACCCGGAGGAAAGCGACGAAGCGGATC
AGGCGCCGTGGACCCCGATGCCGCTGGAGATCGCGTTCCAGGGTGACCGTGAA
AGCGTTCTGAAAGCGATGAACGCGATTACCGGCATGCAAGATTACCTGTTTACC
GTGAACAGCATCCGTATTCGTAACGAACGTATGATGCCGCCGCCAATCGCGAAC
CCGGCTGCGGCGAAGCCGGCTGCGGCGCAGCCGGCGACCGGTGCGGCGAGCC
TGACCCCGGCGGACGAGGCTGCGGCGCCGGCTGCGCCGGCGATCCAGCAAGTT
ATTAAACCGTATATGGGCAAGGAACAGGTGTTCGTTCAAGTGAGCCTGAACCTG

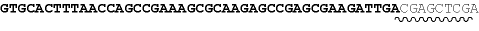

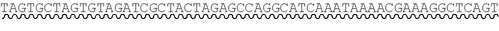

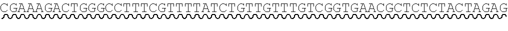

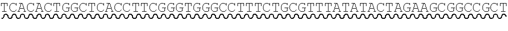

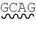

SEQ ID NO: 148
Promoter J23101 RBS usp45 his tag Enterokinase
Amuc_1100_terminator2):

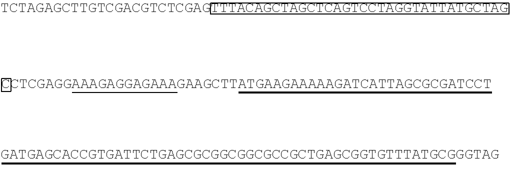

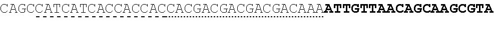

GCGAGCTGGATAAGAAAATCAGCATTGCGGCGAAAGAGATCAAGAGCGCGAAC
GCGGCGGAAATTACCCCGAGCCGTAGCAGCAACGAGGAACTGGAGAAAGAACT
GAACCGTTACGCGAAGGCGGTTGGTAGCCTGGAAACCGCGTATAAACCGTTTCT
GGCGAGCAGCGCGCTGGTGCCGACCACCCCGACCGCGTTCCAAAACGAGCTGA
AAACCTTTCGTGACAGCCTGATCAGCAGCTGCAAGAAAAAGAACATCCTGATTA
CCGATACCAGCAGCTGGCTGGGCTTCCAGGTTTACAGCACCCAAGCGCCGAGC
GTGCAGGCGGCGAGCACCCTGGGTTTTGAGCTGAAAGCGATTAACAGCCTGGT
TAACAAGCTGGCGGAATGCGGCCTGAGCAAATTCATCAAGGTGTATCGTCCGCA
GCTGCCGATTGAAACCCCGGCGAACAACCCGGAGGAAAGCGACGAAGCGGATC
AGGCGCCGTGGACCCCGATGCCGCTGGAGATCGCGTTCCAGGGTGACCGTGAA
AGCGTTCTGAAAGCGATGAACGCGATTACCGGCATGCAAGATTACCTGTTTACC
GTGAACAGCATCCGTATTCGTAACGAACGTATGATGCCGCCGCCAATCGCGAAC
CCGGCTGCGGCGAAGCCGGCTGCGGCGCAGCCGGCGACCGGTGCGGCGAGCC
TGACCCCGGCGGACGAGGCTGCGGCGCCGGCTGCGCCGGCGATCCAGCAAGTT
ATTAAACCGTATATGGGCAAGGAACAGGTGTTCGTTCAAGTGAGCCTGAACCTG

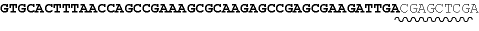

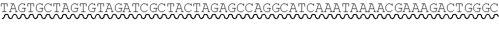

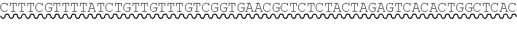

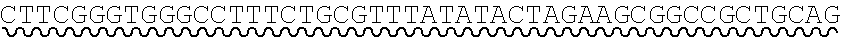

in certain embodiments, the genetically engineered host cell comprises or further comprises more than one copy of Amuc_1100 gene integrated into multiple genome sites of the genetically engineered host cell.

In some embodiments, the integration sites may be any one or more sites selected from the group consisting of agaI/rsmI, araBC, cadA, cadA, dapA, kefB, lacZ, maeB, malE/K, malP/T, rhtB/C, yicS/nepI, adhE, galK, glk, ldhA, lldD, maeA, nth, pflB, rnC, tkrA(ghrB), yieN (ravA), yjcS, and LPP. In some embodiments, the integration sites can be selected from any site as listed in Table 4. In some embodiments, the integration sites are integration sites agaI/rsmI.

In certain embodiments, the genetically engineered host cell comprises or further comprises one or more additional copies of a chaperon gene, or increased expression of the chaperon gene. The chaperon gene can be selected from the group consisting of dsbA, dsbC, dnaK, dnaJ, grpE, groES, groEL, tig, fkpA, surA, skp, PpiD, DegP, and any combination thereof. In certain embodiments, the chaperon genes are selected from the group consisting of dsbA, dsbC, dnaK, dnaJ, grpE, groES, groEL, tig, fkpA, surA, or any combination thereof. In certain embodiments, the genetically engineered host cell comprises one or more chaperon genes selected from the group consisting of dsbA, dsbC, dnaK, dnaJ, grpE, and any combination thereof. In certain embodiments, the genetically engineered host cell comprises one or more chaperon genes selected from the group consisting of groES, groEL, tig, fkpA, surA, and any combination thereof. Some or all of the chaperon genes can be inserted one or more copies in the host cell (e.g. bacterial) genome or their native promoters can be replaced by a stronger promoter in order to increase their expression level.

In certain embodiments, the genetically engineered host cell comprises or further comprises an inactivation or deletion in a gene encoding outer membrane protein. The outer membrane protein is LPP.

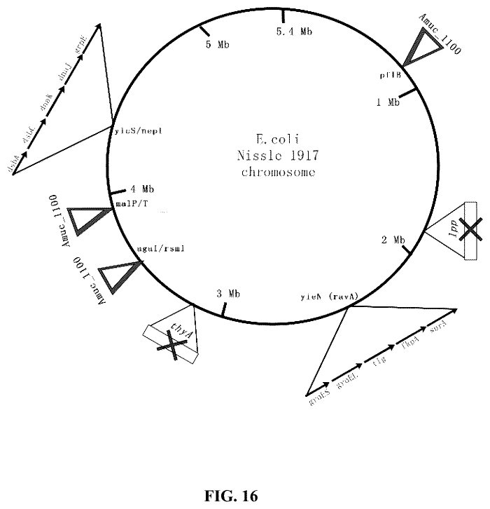

FIG. 16 shows an example of one genetically engineered and optimized EcN that encoding Amuc_1100 gene cassette. In this engineered strain, Amuc_1100 gene regulatory elements including promoter, RBS, cistron, and element encoding signal peptide were adjusted. Three copies of Amuc_1100 gene cassette were inserted into different sites (agaI/rsmI, malPT and pflB), the host EcN was modified by inserting chaperon genes including dsbA, dsbC, dnaK, dnaJ, grpE, groES, groEL, tig, fkpA, and surA, and also by deleting 1pp and thyA genes.

C. Immune Checkpoint Modulators

Immune checkpoints are regulators of the immune system, and belong to immunoinhibitory pathway or immunostimulatory pathway, responsible for co-stimulatory or inhibitory interactions of T-cell responses, and regulate and maintain self-tolerance and physiological immune responses.

As used herein, the term “immunostimulatory pathway” refers generally to a signaling pathway that has a stimulatory effect on the immune system in the host, which can positively modulate cytokine secretion, NK cell activation, T cell proliferation, and antibody production, etc. Immunostimulatory checkpoint molecules used herein can be any known or later discovered immunostimulatory checkpoint molecules. Non-limiting immunostimulatory checkpoint molecules found in the immunostimulatory pathways can include CD2, CD3, CD7, CD16, CD27, CD30, CD70, CD83, CD28, CD80 (B7-1), CD86 (B7-2), CD40, CD40L (CD154), CD47, CD122, CD137, CD137L, OX40 (CD134), OX40L (CD252), NKG2C, 4-1BB, LIGHT, PVRIG, SLAMF7, HVEM, BAFFR, ICAM-1, 2B4, LFA-1, GITR, ICOS (CD278), or ICOSLG (CD275), among others.

As used herein, the term “immunoinhibitory pathway” refers generally to a signaling pathway in a host that has an inhibitory effect on the immune system in the host, which can negatively module cytokine secretion, NK cell activation, T cell proliferation, and antibody production, etc. Immunoinhibitory checkpoint molecules used herein can be any known or later discovered immunoinhibitory checkpoint molecules. Non-limiting immunoinhibitory checkpoint molecules found in the immunoinhibitory pathways can include LAG3 (CD223), A2AR, B7-H3 (CD276), B7-H4 (VTCN1), BTLA (CD272), BTLA, CD160, CTLA-4 (CD152), IDO1, IDO2, TDO, KIR, LAIR-1, NOX2, PD-1, PD-L1, PD-L2, TIM-3, VISTA, SIGLEC-7 (CD328), TIGIT, PVR (CD155), TGFβ, or SIGLEC9 (CD329), among others.

As used herein, the term “immune checkpoint modulator” refers to a molecule that modulates (e.g., totally or partially reduces, inhibits, interferes with, activates, stimulates, increases, reinforces or supports) the function of one or more immune checkpoints within the immunostimulatory or immunoinhibitory pathways.

In some embodiments, the immune checkpoint modulator provided herein comprises an immunoactivator that can totally or partially activates, stimulates, increases, reinforces, supports or positively modulates the function of one or more immune checkpoints within the immunostimulatory pathway, or can totally or partially reduces, inhibits, interferes with, or negatively modulates the function of one or more immune checkpoints within the immunoinhibitory pathways.

Immune checkpoint modulators are typically able to modulate (i) self-tolerance and/or (ii) the amplitude and/or the duration of the immune response. Preferably, the immune checkpoint modulator used in the present disclosure modulates the function of one or more human checkpoint molecules and is, thus, a “human immune checkpoint modulator”.

In some embodiments, the immune checkpoint modulator comprises an activator of one or more immunostimulatory checkpoints selected from the group consisting of CD2, CD3, CD7, CD16, CD27, CD30, CD70, CD83, CD28, CD80 (B7-1), CD86 (B7-2), CD40, CD40L (CD154), CD47, CD122, CD137, CD137L, OX40 (CD134), OX40L (CD252), NKG2C, 4-1BB, LIGHT, PVRIG, SLAMF7, HVEM, BAFFR, ICAM-1, 2B4, LFA-1, GITR, ICOS (CD278), ICOSLG (CD275), and any combination thereof.

In some embodiments, the immune checkpoint modulator comprises an inhibitor of one or more immunoinhibitory checkpoints selected from the group consisting of LAG3 (CD223), A2AR, B7-H3 (CD276), B7-H4 (VTCN1), BTLA (CD272), BTLA, CD160, CTLA-4 (CD152), IDO1, IDO2, TDO, KIR, LAIR-1, NOX2, PD-1, PD-L1, PD-L2, TIM-3, VISTA, SIGLEC-7 (CD328), TIGIT, PVR (CD155), TGFβ, SIGLEC9 (CD329), and any combination thereof.

Details of these above mentioned modulators in the immunostimulatory and immunoinhibitory pathways are known in the art, see, e.g., WO 97/28259; WO 98/16247; WO 99/11275; Krieg A M et al., 1995; Yamamoto S et al. ,1992; Ballas Z K et al., 1996; Klinman D M et al., 1997; Sato Y et al., 1996; Pisetsky D S, 1996; Shimada S et al., 1986; Cowdery J S et al., 1996; Roman H et al., 1997; Lipford G B et al., 1997; WO 98/55495 and WO 00/61151, etc.

In some embodiments, the immune checkpoint modulator of the present disclosure is an antibody or an antigen-binding fragment thereof or a chemical compound against the immune checkpoints of the present disclosure. Non-limiting examples of antibodies against the immune checkpoints include anti-PD-1 antibodies (including but not limited to Nivolumab, Pidilizumab, Pembrolizumab (MK-3475/SCH900475, lambrolizumab, REGN2810, PD-1 (Agenus)), anti-PD-L1 antibodies (including but not limited to durvalumab (MEDI4736), avelumab (MSB0010718C), and atezolizumab (MPDL3280A, RG7446, R05541267)), anti-PDL-2 antibodies (including, but not limited to, AMP-224 (described in WO2010027827 and WO2011066342)), CTLA-4 antibodies (including but not limited to Ipilimumab and Tremelimumab (CP675206)), anti-4-1BB antibodies (including but not limited to PF-05082566, and Urelumab), LAG3 antibodies (including but not limited to BMS-986016), anti-CD27 antibodies (including but not limited to Varlilumab).

In some embodiments, in the methods provided herein, more than one immune checkpoint modulators (e.g. anti-PD-1 antibody and anti-PD-L1 antibody) are used.

In some embodiments, the immune checkpoint modulator modulates (or inhibits) an immunoinhibitory checkpoint. In some embodiments, the immunoinhibitory checkpoint is PD-L1, PD-L2, or PD-1. In some embodiments, the immune checkpoint modulator inhibits interaction between PD-1 and any of its ligands. In some embodiments, the immune checkpoint modulator comprises an antibody (e.g. a human antibody, a humanized antibody, or a chimeric antibody) or an antigen-binding fragment thereof, or a chemical compound that inhibits or reduces interaction between PD-1 and any of its ligands or the signaling (e.g. interaction between PD-L1/PD-L2, or PD-1). In some embodiments, the immune checkpoint modulator comprises an anti-PD-1 antibody, anti-PD-L1 antibody, anti-PD-L2 antibody, a small-molecule PD-1 inhibitor, a small-molecule PD-L1 inhibitor, or a small-molecule PD-L2 inhibitor.

In some embodiments, the antibody described herein (such as an anti-PD-1 antibody, an anti-PD-L1 antibody, or an anti-PD-L2 antibody) further comprises a human or murine constant region. In some embodiments, the human constant region is selected from the group consisting of IgG1, IgG2, IgG2, IgG3, IgG4. In some embodiments, the human constant region is IgG1. In some embodiments, the murine constant region is selected from the group consisting of IgG1, IgG2A, IgG2B, IgG3. In some embodiments, the antibody has reduced or minimal effector function. In some embodiments, the minimal effector function results from production in prokaryotic cells. In some embodiments, the minimal effector function results from an “effector-less Fc mutation” or aglycosylation.

Accordingly, an antibody used herein can be glycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxy amino acid, most commonly serine or threonine, although 5-hydroxyproline or 5-hydroxy lysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

The antibody or antigen binding fragment thereof, may be made using methods known in the art, for example, by a process comprising culturing a host cell containing nucleic acid encoding any of the previously described anti-PD-1, anti-PD-LI, or anti-PDL2 antibodies or antigen-binding fragment in a form suitable for expression, under conditions suitable to produce such antibody or fragment, and recovering the antibody or fragment.

The chemical compound against the immune checkpoint of the present disclosure may be a small molecule compound. The term “small molecule compound” as used herein means a low molecular weight compound that may serve as an enzyme substrate or regulator of biological processes. In general, a “small molecule compound” is a molecule that is less than about 5 kilodaltons (kD) in size. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. In some embodiments, in accordance with the present disclosure, small molecules are not proteins, polypeptides, oligopeptides, peptides, polynucleotides, oligonucleotides, polysaccharides, glycoproteins, proteoglycans, etc. In some embodiments, a small molecule is a therapeutic. In some embodiments, a small molecule is an adjuvant. In some embodiments, a small molecule is a drug.

In some embodiments, the subject has shown poor response, or resistance to the immune checkpoint modulator. “Poor response” or “resistance to” the immune checkpoint modulator refers to the subject who is receiving the treatment of immune checkpoint modulator does not respond to, or poorly respond to the treatment, and thereby the disease, disorder or condition of the subject is not being treated. The term “resistance” as used herein refers to being refractory or relapsed or non-responsive to a therapeutic agent, such as an immune checkpoint modulator. The subject's response to the treatment of immune checkpoint modulator can be determined by means known in the state of the art.

In some embodiments, the subject is determined to have de novo or acquired resistance to the immune checkpoint modulator. “De novo resistance” as used herein refers to resistance that exists prior to treatment with a given agent. Therefore, “de novo resistance to an immune checkpoint modulator” means the subject is resistant or non-responsive to the immune checkpoint modulator prior to the treatment of the immune checkpoint modulator. “Acquired resistance” as used herein refers to resistance that is acquired after at least one treatment with a given agent. Prior to the at least one treatment, the disease does not possess a resistance to the agent (and, as such, the disease responds to the first treatment as would a non-resistant disorder). For example, a subject who has acquired resistance to the immune checkpoint modulator is one that initially responds to at least one treatment of the immune checkpoint modulator and thereafter develops a resistance to subsequent treatments of the immune checkpoint modulator.

D. Indications

As mentioned above, the present disclosure provides a method of treating or preventing cancer in a subject in need thereof, the present disclosure also provides a method of improving therapeutic response to cancer in a subject receiving treatment of an immune checkpoint modulator. The terms “cancer” and “tumor” are used interchangeably herein and refer to a disease, disorder or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, such cells exhibit such characteristics in part or in full due to the expression and activity of oncogenes or the defective expression and/or activity of tumor suppressor genes, such as retinoblastoma protein (Rb). Cancer cells are often in the form of a tumor, but such cells may exist alone within an animal, or may be a non-tumorigenic cancer cell, such as a leukemia cell. As used herein, the term “cancer” includes premalignant as well as malignant cancers.

The phrase “improving therapeutic response” can include, for example, delaying progression of a disease or reducing or inhibiting cancer relapse.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

In some embodiments, the cancer of the present disclosure is selected from the group consisting of colorectal cancer, breast cancer, ovarian cancer, pancreatic cancer, gastric cancer, prostate cancer, pancreatic cancer, renal cancer, cervical cancer, myeloma cancer, lymphoma cancer, leukemia cancer, thyroid cancer, endometrial cancer, uterine cancer, bladder cancer, neuroendocrine cancer, head and neck cancer, liver cancer, nasopharyngeal cancer, testicular cancer, lung cancer (e.g. small cell lung cancer, non-small cell lung cancer), melanoma, basal cell cancer, skin cancer, squamous cell skin cancer, dermatofibrosarcoma protuberans, Merkel cell carcinoma, glioblastoma, glioma, sarcoma and mesothelioma. In some embodiments, the cancer is breast cancer.

As mentioned above, the present disclosure also provides a method of inducing or improving gut immunity in a subject (e.g. human). Gut microbiota has been inhabited with human over millions of years. Bacteria and human have established a relevantly stable and commensal evolutionary relationship. This relation renders gut bacteria the functions to regulate and shape gut immune system and physiology. Gut microbiota dysbiosis has been correlated with a myriad of diseases including cancer, metabolic disease, autoimmune disease, cardiovascular disease, neurodegenerative disease, liver disease, and so on. Recent evidences suggest that gut microbiota composition especially certain bacteria strains can predict or increase the efficacy of cancer immunotherapy. Although many detailed molecular mechanisms controlling the complex interactions between gut immunity and gut microbiota remain to be broken, the present inventors unexpectedly found that the combination of Amuc_1100 (or a genetically engineered host cell expressing the Amuc_1100) and an immune checkpoint modulator can synergistically induce or improve gut immunity in a subject.

E. Combination

The Amuc_1100 or genetically engineered host cell expressing the Amuc_1100 provided herein may be administered by any route known in the art, such as for example parenteral (e.g. subcutaneous, intraperitoneal, intravenous, including intravenous infusion, intramuscular, or intradermal injection) or non-parenteral (e.g. oral, intranasal, intraocular, sublingual, rectal, or topical) routes. In some embodiments, the administration is via oral administration.

In some embodiments, the Amuc_1100 or genetically engineered host cell expressing the Amuc_1100 provided herein that is administered in combination with the immune checkpoint modulator may be administered simultaneously with the immune checkpoint modulator, and in certain of these embodiments the Amuc_1100 or genetically engineered host cell expressing the Amuc_1100 and the immune checkpoint modulator may be administered as part of the same pharmaceutical composition. However, Amuc_1100 or genetically engineered host cell expressing the Amuc_1100 administered “in combination” with an immune checkpoint modulator does not have to be administered simultaneously with or in the same composition as the agent. Amuc_1100 or genetically engineered host cell expressing the Amuc_1100 administered prior to or after the immune checkpoint modulator is considered to be administered “in combination” with the immune checkpoint modulator as the phrase is used herein, even if the Amuc_1100 or genetically engineered host cell expressing the Amuc_1100 and the immune checkpoint modulator are administered via different routes (for example, the Amuc_1100 or genetically engineered host cell expressing the Amuc_1100 is administered orally, while the immune checkpoint modulator is administered by injection). For example, the Amuc_1100 (or genetically engineered host cell expressing the Amuc_1100) may be administered prior to the immune checkpoint modulator (e.g. 5 hours, 10 hours, 12 hours, 24 hours, 48 hours, 72 hours, 96 hours, 1 week, 2 weeks, 3 weeks, etc.), either punctually or several times before the immune checkpoint modulator is administered, as long as they exert effects in the subject during an overlapped timeframe. For another example, the Amuc_1100 (or genetically engineered host cell expressing the Amuc_100) is administered prior to the immune checkpoint modulator for a certain period of time (e.g. 5 hours, 10 hours, 12 hours, 24 hours, 48 hours, 72 hours, 96 hours, 1 week, 2 week, 3 weeks, etc.), and then the Amuc_1100 (or genetically engineered host cell expressing the Amuc_1100) and the immune checkpoint modulator are administered simultaneously. Where possible, immune checkpoint modulator(s) administered in combination with the Amuc_1100 or genetically engineered host cell expressing the Amuc_1100 disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

In some embodiments, the present disclosure also provides a nucleic acid vector comprising the recombinant expression cassette of the present disclosure for use in combination with an immune checkpoint modulator.

In another aspect, the present disclosure also provides a genetically engineered host cell of the present disclosure for use in combination with an immune checkpoint modulator.

III. Kits

In another aspect, the present disclosure provides a kit comprising: (a) a first composition comprising the genetically engineered host cell of the present disclosure or comprising an isolated Amuc_1100; and (b) a second composition comprising an immune check point inhibitor.

In some embodiments, the first composition is an edible composition, and/or the second composition further comprises a pharmaceutically acceptable carrier. In some embodiments, the first composition is a food supplement. In some embodiments, the second composition is suitable for oral administration or parenteral administration.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or as labels, indicating quantities of the components to be administered, guidelines for administration, and/or guidelines for mixing the components, can also be included in the kit.

IV. Compositions

A. Composition Comprising Amuc 1100 or Functional Equivalents Thereof

In one aspect, the present disclosure provides a composition comprising the Amuc_1100 or functional equivalents thereof of the present disclosure, and a physiologically acceptable carrier.

In some embodiments, the Amuc_1100 comprises an Amuc_1100 polypeptide. In some embodiments, the Amuc_1100 polypeptide is a recombinant Amuc_1100 polypeptide. In some embodiments, the Amuc_1100 polypeptide is a purified recombinant Amuc_1100 polypeptide. In some embodiments, the purity of the purified recombinant Amuc_1100 polypeptide is at least 80%, 85%, 90%, 91%, 92%, 93%, 94%, 95%, 96%, 97%, 98%, 99%, 99.5%, or 99.9% by weight.

Physiologically acceptable carriers for use in the compositions disclosed herein may include, for example, physiologically acceptable liquid, gel, or solid carriers, aqueous vehicles, nonaqueous vehicles, antimicrobial agents, isotonic agents, buffers, antioxidants, suspending/dispending agents, sequestering or chelating agents, diluents, adjuvants, excipients, or non-toxic auxiliary substances, other components known in the art, or various combinations thereof.

To further illustrate, the aqueous vehicles may include such as sodium chloride injection, Ringer's injection, isotonic dextrose injection, sterile water injection, or dextrose and lactated Ringer's injection; nonaqueous vehicles may include such as fixed oils of vegetable origin, cottonseed oil, corn oil, sesame oil, or peanut oil; antimicrobial agents may be at bacteriostatic or fungistatic concentrations, and/or may be added to the compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Isotonic agents may include such as sodium chloride or dextrose; buffers may include such as phosphate or citrate buffers; antioxidants may include such as sodium bisulfate; suspending and dispersing agents may include such as sodium carboxymethylcelluose, hydroxypropyl methylcellulose, or polyvinylpyrrolidone; sequestering or chelating agents may include such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

In some embodiments, the compositions provided herein may be a pharmaceutical composition. In some embodiments, the compositions provided herein may be a food supplement. The compositions provided herein may comprise a pharmaceutical, nutritionally or alimentarily or physiologically-acceptable carrier, in addition to the Amuc_100 provided herein. The preferred form will depend on the intended mode of administration and (therapeutic) application. The carrier may be any compatible, physiologically-acceptable, non-toxic substances suitable to deliver the Amuc_1100 provided herein to the GI tract of a mammal (e.g. human), preferably in the vicinity of or within the gut mucosal barrier (more preferably the colon mucosal barrier) in a mammal.

The compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

B. Composition Comprising the Genetically Engineered Host Cell Expressing the Amuc 1100 or Functional Equivalents Thereof

In another aspect, the present disclosure also provides a composition comprising the genetically engineered host cell expressing the Amuc_1100 or functional equivalents thereof, and a physiologically acceptable carrier. The host cell may be present in the composition in an amount ranging from about 104 to about 10¹³ or about 104 to about 10¹⁵ colony forming units (CFUs). For instance, an effective amount of the host cell may be an amount of about 10⁵ CFUs to about 10¹³ or about 10¹⁴ CFUs, preferably about 10⁶ CFUs to about 10¹³ CFUs, preferably about 10⁷ CFUs to about 10¹² CFUs, more preferably about 108 CFUs to about 10¹² CFUs. The host cell may be viable or may be dead. The effectiveness of the host cell correlates with the presence of the Amuc_1100 provided herein.

In some embodiment, the compositions disclosed herein can be formulated for oral administration and may be a nutritional, or alimentary, composition, for example, a food, food supplement, feed, or a feed supplement such as a dairy product, e.g., a fermented dairy product, such as a yogurt or a yogurt drink. In this case, the composition may comprise a nutritionally acceptable or alimentarily acceptable carrier, which may be a suitable food base. The skilled artisan knows a variety of formulas which can encompass living or killed microorganisms and which can present as food supplements (e.g., pills, tablets and the like) or as functional food such as drinks, fermented yoghurts, etc.. The compositions disclosed herein can also be formulated as medicaments, in capsules, pills, liquid solution, for example as encapsulated lyophylized bacteria etc..

The compositions disclosed herein may be formulated to be effective in a given subject in a single administration or over multiple administrations. For example, a single administration is substantially effective to reduce a monitored symptom of a targeted disease condition in a mammalian subject to whom the composition is administered.

In some embodiments, the composition is formulated such that a single oral dose contains at least or at least about 1×10⁴ CFU of the bacterial entities and/or fungal entities, and a single oral dose will typically contain about or at least 1×10⁴, 1×10⁵, 1×10⁶, 1×10⁷, 1×10, 1×10⁹, 1×10¹⁰, 1×10¹¹, 1×10¹², 1×10¹³, 1×10¹⁴, 1×10¹⁵, or greater than 1×10¹⁵ CFUs of the bacterial entities and/or fungal entities. If known, for example the concentration of cells of a given strain, or the aggregate of all strains, is e.g., 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵viable bacterial entities (e.g., CFUs) per gram of composition or per administered dose.

In some formulations, the composition contains at least or at least about 0.5%, 1%, 2%, 5%, 10%, 20%, 30%, 40%, 50%, 60%, 70%, 80%, 90% or greater than 90% host cells of the present disclosure on a mass basis. In some formulations, the administered dose does not exceed 200, 300, 400, 500, 600, 700, 800, 900 milligrams or 1, 1.1, 1.2, 1.3, 1.4, 1.5, 1.6, 1.7, 1.8, or 1.9 grams of host cells of the present disclosure in mass.

C. Composition Comprising the Amuc 1100 (or Genetically Engineered Host Cell Disclosed Herein) and an Immune Checkpoint Modulator

In another aspect, the present disclosure provides a composition comprising: (a) the genetically engineered host cell of the present disclosure or an isolated Amuc_1100 of the present disclosure; and (b) an immune check point inhibitor of the present disclosure. In some embodiments, the composition is edible.

In another aspect, the present disclosure provides a composition comprising the genetically engineered host cell of the present disclosure or an isolated Amuc_1100 for use in combination with a formulation comprising an immune checkpoint modulator of the present disclosure. In some embodiments, the composition can be administered to the subject by any route known in the art, such as for example parenteral (e.g. subcutaneous, intraperitoneal, intravenous, including intravenous infusion, intramuscular, or intradermal injection) or non-parenteral (e.g. oral, intranasal, intraocular, sublingual, rectal, or topical) routes.

In some embodiments, the composition is an oral composition. In some embodiments, the formulation comprising an immune checkpoint modulator is a parenteral (e.g. subcutaneous, intraperitoneal, intravenous, including intravenous infusion, intramuscular, or intradermal injection) formulation. In some embodiments, the formulation comprising an immune checkpoint modulator is an oral formulation.

In another aspect, the present disclosure provides use of the composition comprising Amuc_1100 (or a genetically engineered host cell expressing the Amuc_1100) in the manufacture of a medicament for treating or preventing cancer.

In another aspect, the present disclosure provides use of the composition comprising Amuc_1100 (or a genetically engineered host cell expressing the Amuc_1100) in the manufacture of a medicament for improving therapeutic response to cancer in a subject who receiving treatment of an immune checkpoint modulator.

In another aspect, the present disclosure provides use of the composition comprising Amuc_1100 (or a genetically engineered host cell expressing the Amuc_1100) in the manufacture of a medicament for inducing or improving gut immunity in a subject.

In another aspect, the present disclosure provides use of the composition comprising Amuc_1100 (or a genetically engineered host cell expressing the Amuc_1100) in the manufacture of a medicament for improving therapeutic response to cancer in a subject receiving an anti-cancer treatment and showing decrease in gut immunity.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

V. Amuc 1100 Mutant

Also provided in the present disclosure is a non-naturally occurring Amuc_100 protein. In certain embodiment, the Amuc_1100 polypeptides comprises one or more (e.g. 2, 3, 4, 5, 6, 7, 8, 9, 10, 11, 12, 13, 14, 15, 16, 17, 18, 19, 20, 21, 22, 23, 24, 25 or more) mutations at a position selected from the group consisting of: R36, S37, L39, D40, K41, K42, 143, K48, E49, K51, S52, R62, S63, K70, E71, L72, N73, R74, Y75, A76, K77, A78, Y86, K87, P88, F89, L90, A91, F103, Q104, K108, T109, F110, R111, D112, K119, K120, K121, N122, L124, 1125, W131, L132, G133, F134, Q135, Y137, 5138, L150, G151, F152, E153, L154, K155, A156, L160, V161, K163, L164, A165, L169, S170, K171, F172, I173, K174, V175, Y176, R177, W200, T201, L205, E206, F209, Q210, R213, E214, L217, K218, A219, M220, N221, Y229, L230, R237, 1238, R242, M243, M244, P245, K255, P256, L268, T269, K287, P288, Y289, M290, K292, E293, F296, V297, F306, N307, K310 and A311, wherein the numbering is relative to SEQ ID NO: 1.

In certain embodiments, the Amuc_1100 polypeptide comprises one or more (e.g. 2, 3, or 4) mutations at a position selected from the group consisting of: S37, V175, Y289 and F296, wherein the numbering is relative to SEQ ID NO: 1. In certain embodiments, the Amuc_1100 polypeptide comprises Y289A mutation, wherein the numbering is relative to SEQ ID NO: 1. In certain embodiments, the Amuc_100 polypeptide comprises an amino acid sequence of SEQ ID NO: 4 or SEQ ID NO: 5.

The non-naturally occurring Amuc_100 proteins provided herein, for example, the Amuc_1100 mutant comprising Y289A mutation as mentioned above, exhibit higher resistance to enzymatic digestion than naturally-occurring counterparts, and hence would be advantageous for use in the engineered bacteria expressing Amuc_1100 provided herein in an environment having enzymes that may degrade Amuc_1100 once it is secreted from the engineered bacteria and released into the environment.

In another aspect, the present disclosure also provides a nucleic acid encoding the non-naturally occurring Amuc_1100 proteins as mentioned above. The nucleic acid encoding the non-naturally occurring Amuc_1100 proteins may be obtained and/or optimized by the person skilled in the art using various publicly available tools such as tblastn (available on the website of U.S. National Center for Biotechnology Information (NCBI). A person skilled in the art may use the default parameters provided by the tool or may customize the parameters as appropriate for the blast, such as for example, by selecting a suitable algorithm.

VI. Usp45 Signal Peptide

The present disclosure also provides use of Usp45 signal peptide in the construction of an expression vector comprising a polynucleotide encoding a target polypeptide, wherein the expression vector is suitable for expression in E. Coli to allow expression and secretion of the target polypeptide from the E. Coli.

Also provided herein is Usp45 signal peptide, for use in the construction of an expression vector comprising a polynucleotide encoding a target polypeptide, wherein the expression vector is suitable for expression in E. Coli to allow expression and secretion of the target polypeptide from the E. Coli.

Also provided herein is an expression vector comprising a polynucleotide encoding a target polypeptide operably linked to Usp45 signal peptide, wherein the expression vector is suitable for expression in E. Coli to allow expression and secretion of the target polypeptide from the E. Coli.

In another aspect, the present disclosure also provides a genetically engineered E. coli, comprising an exogenous expression cassette comprising polynucleotide encoding a target polypeptide operably linked to Usp45 signal peptide. The genetically engineered E. coli as provided herein is capable of expressing and secreting the target polypeptide.

In certain embodiment, the Usp45 signal peptide mentioned above comprises the sequence as set forth in SEQ ID NO: 59. In certain embodiment, the E. Coli is a commensal strain of E. Coli, for example, E. coli Nissle 1917 strain.

The target polypeptide may comprise Amuc_1100 provided herein. The target polypeptide may also comprise a polypeptide other than Amuc_1100.

Usp45 signal peptide is the signal peptide of the major secreted protein of Lactococcus lactis, and it is the most-used signal peptide for protein secretion in Lactococcus lactis (see, for example, Jhonatan et al., Appl Environ Microbiol. 2020 August; 86(16): e00903-20.). Generally, Usp45 signal peptide are considered to merely work in Lactococcus lactis and the ability to direct secretion of proteins in E. coli has never been contemplated or explored. Accordingly, the use of the Usp45 signal peptide in E. coli, such as E. coli Nissle 1917 strain, and the engineered E. coli, such as E. coli Nissle 1917 strain that uses Usp45 as a signal peptide for secreting a target polypeptide, are novel and moreover have achieved unexpectedly high secretion of the target polypeptide (e.g., Amuc_100) from E. coli, such as E. coli Nissle 1917 strain.

### EXAMPLES

### Example 1. Production of Recombinant Amuc 1100 and its Mutants

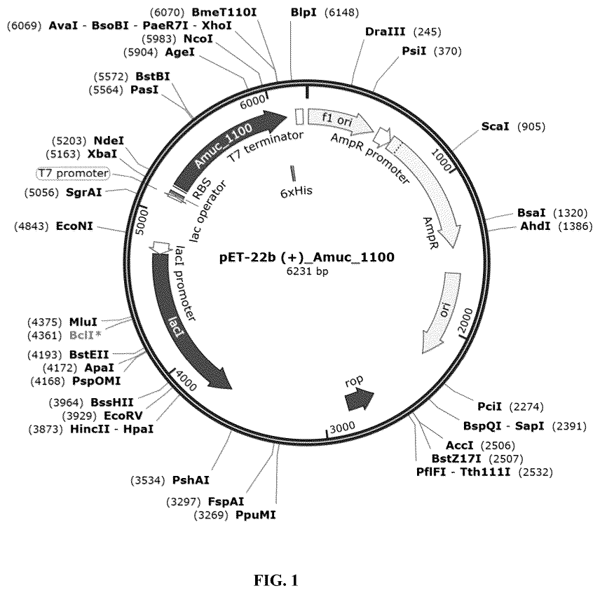

DNA sequences that were code-optimized for Amuc_1100 (31-317) were constructed in plasmid pET-22b(+) (named as “pET-22b (+)_Amuc_1100”, as shown in FIG. 1) at restriction enzyme sites NdeI and XhoI. The plasmid was then transformed into E. coli BL21 (DE3). The transformed bacteria were cultured in LB medium at 37° C. to cell density at OD600 value of 0.8. Amuc_1100 (31-317) expression was induced by the lactose analog IPTG (0.1 mM) overnight at 16° C., or induced by IPTG (1 mM) for 3h at 37° C. Protein expression was detected by SDS-PAGE gel and Coomassie blue staining.

The Amuc_1100 (31-317) expression induced bacteria were collected and resuspended in buffer (20 mM PB, 500 mM NaCl, pH 7.4) and lysed by ultrasonication. The supernatant of cell lysate was purified by Chelating SFF (Ni) column and eluted by Imidazole-containing buffer. The Amuc_1100 collected from Chelating SFF (Ni) column were enriched by Q-HP column and eluted by buffer (20 mM PB, 500 mM NaCl, pH 7.4) containing reduced concentration of NaCl. The purified recombinant Amuc_1100 (31-317) was digested by mixture of Trypsin and Chymotrypsin in non-reduced condition in PBS at 37° C. for 30 min. Samples were analyzed by SDS-PAGE gel and Coomassie blue staining.

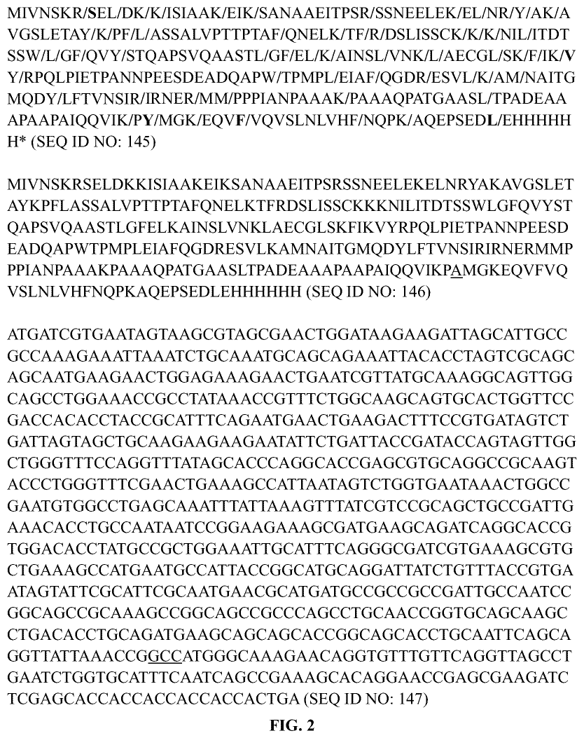

Trypsin and Chymotrypsin-digested Amuc_1100 (31-317) was then subjected to MS analysis for detecting the digestion sites at the protein. As shown in SEQ ID NO: 145 of FIG. 2, the amino acids next to the label “/” are susceptible sites for the enzyme digestion. Particularly, Ser37, Val175, Tyr289, and Phe296 (the numbering is relative to SEQ ID NO: 1) were highlighted as sensitive sites for the enzyme digestion.

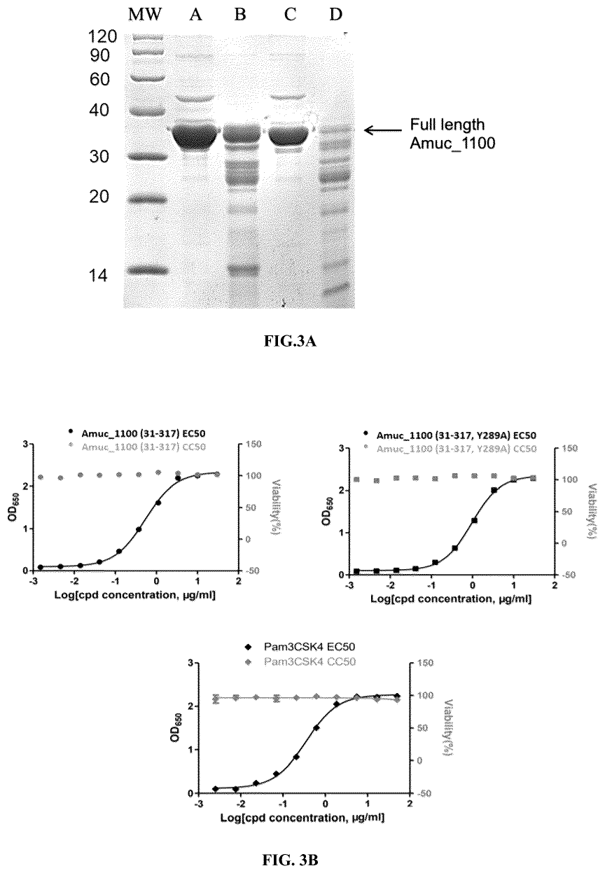

Some mutants of the Amuc_100 (31-317) may reduce protein degradation by proteases. The amino acid sequence (SEQ ID NO: 146) and nucleotide sequence (SEQ ID NO: 147) of Amuc_1100 (31-317, Y289A) with 6×His tag at the C-terminus expressed by the plasmid described above (FIG. 1) were shown in FIG. 2. The recombinant Amuc_1100 (31-317) protein and mutant protein (31-317, Y289A) were digested by proteases trypsin (0.01 mg/ml) and α-chymotrypsin (0.01 mg/ml) at 37° C. for 30 min. The samples were then subjected to SDS-PAGE gel electrophoresis and Coomassie blue staining. As shown in FIG. 3A, the Amuc_1100 (31-317, Y289A) protein were more resistant to enzyme digestion compared to wild type Amuc_1100 protein, which indicates a single point mutation Y289A could confer Amuc_1100 a greater stability.

HEK293 cells expressing human TLR2 receptor and its reporter gene (InvivoGen, hkb-htlr2) were seeded in a 96-well plate and treated by Amuc_100 (31-317) or mutant recombinant Amuc_1100 (31-317, Y289A). The reporter gene activity was detected by QUANTI-Blue™ (InvivoGen, rep-qbs2), and the cell viability was detected by CellTiterGlo (Promega, G7573) by following the reagent kits' instruction. The results were shown in FIG. 3B (Pam3CSK4 is the positive control for TLR2 ligand). As shown in FIG. 3B, the EC₅₀ of Amuc_1100 (31-317), Amuc_1100 (31-317, Y289A) and Pam3CSK4 for TLR2 activation are 0.535 μg/ml, 0.951 μg/ml, 0.351 μg/ml respectively. All agents show no significant impact on cell viability.

### Example 2. Use of Amuc_1100 (31-317) or its Mutants in Cancer Treatment

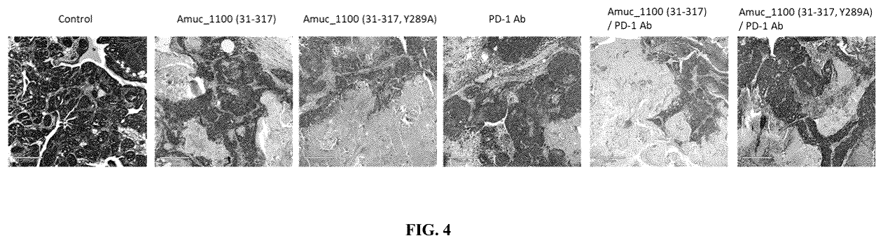

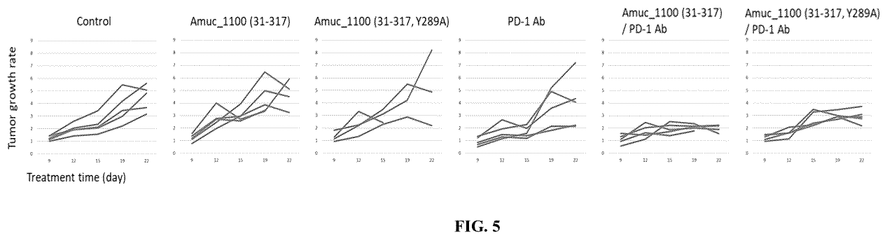

The MMTV-PyVT transgenic C57BL/6 female mice were used to develop spontaneous breast cancer. Mice at age of 9.5 weeks were randomly divided into 6 groups with 4-5 mice in each group and were subjected to agent treatment. The mice in the control group were treated with PBS by oral gavage daily for 4 weeks, plus PBS i.v. injection daily after one week of oral gavage for 3 weeks. The mice in the five test groups were treated with (1) Amuc_1100 (31-317) protein in PBS (3 μg/kg) by oral gavage daily for 4 weeks, (2) Amuc_1100 (31-317, Y289A) protein in PBS (3 μg/kg) by oral gavage daily for 4 weeks, (3) anti-PD-1 antibody (InVivoMAb anti-mouse PD-1 (CD279) (Clone RMP1-14) (BioXcell, catalog #BE0146)) (after one week of oral gavage of PBS) by i.v. injection(10 mg/kg) every three days for 3 weeks, (4) combination of Amuc_1100 (31-317) protein and anti-PD-1 antibody as described above, i.e. Amuc_1100 (31-317) (3 μg/kg) by oral gavage daily for one week, and then anti-PD-1 antibody (10 mg/kg, i.v. injection every three days) simultaneously with Amuc_1100 (31-317) (oral gavage daily) for another 3 weeks, (5) combination of Amuc_1100 (31-317, Y289A) protein and anti-PD-1 antibody as described above, i.e. Amuc_1100 (31-317, Y289A) (3 μg/kg) by oral gavage daily for one week, and then anti-PD-1 antibody (10 mg/kg, i.v. injection every three days) simultaneously with Amuc_1100 (31-317, Y289A) (3 μg/kg) (oral gavage daily) for another 3 weeks. All the spontaneously developed tumors were monitored. Tumor sizes were measured twice every week. The health status of mice was closely monitored. Tumor growth rate of each tumor in mouse following agent treatment was analyzed. At endpoint of the study, tumor tissues were collected and fixed in formalin. H&E staining was performed to determine the tumor cells (dark staining) in the tissue. The staining results were shown in FIG. 4, and the average tumor growth rate of all tumors in each mouse in each group was shown in FIG. 5.

As shown in FIG. 4 and FIG. 5, the Amuc_1100 (31-317) protein, the Amuc_1100 (31-317, Y289A) protein and the anti-PD-1 antibody partially inhibited tumor growth, while the combination of the Amuc_100 (31-317) protein and the anti-PD-1 antibody, and the combination of the Amuc_100 (31-317, Y289A) protein and the anti-PD-1 antibody synergistically inhibited tumor growth, i.e. significantly more potent than either treatment alone.

The use of Amuc_100 (31-317) or Amuc_1100 (Y289A) alone or in combination with anti-PD-1 antibody in the treatment of colorectal cancer mice model (established by subcutaneous injection of MC-38 cells into C57BL/6 mice) is also tested, and similar synergistic effect between Amuc_1100 protein and anti-PD-1 antibody can be observed as well.

### Example 3. Impact of Amuc_1100 or its Mutants on Body Weight Gain in Mouse Model

Mice at age of 9.5 weeks were randomly divided into 3 groups with 4-5 mice in each group and were subjected to agent treatment. The mice in the control group and test groups were treated with PBS, Amuc_1100 (31-317), or Amuc_1100 (31-317, Y289A) (3 μg/kg) by oral gavage daily for 4 weeks, respectively. The body weight of each mouse in each group was measured twice every week. The results showed that, compared to the control group, no significant changes in body weights were observed in the mice of the test group (i.e. treated with Amuc_1100 (31-317) or Amuc_1100 (31-317, Y289A).

### Example 4. Impact of Amuc_1100 or its Mutants on Treg Infiltration

The MMTV-PyVT transgenic C57BL/6 female mice are used to develop spontaneous breast cancer. Mice at age of 9.5 weeks are randomly divided into 3 groups with 4-5 mice in each group and are subjected to agent treatment. The mice in the control group and test groups are treated with PBS, Amuc_1100 (31-317), or Amuc_1100 (31-317, Y289A) by oral gavage daily for 4 weeks, respectively. At endpoint of the study, tumor tissues are collected and fixed in formalin. H&E staining or immunofluorescence assay is performed to determine the distribution of immune cells (including macrophages, Tregs, CD4+ T cells, CD8+ T cells, etc.) in the tumor tissues.

### Example 5. Bacteria Genome Editing Protocol

5.1 sgRNA design

A 20-bp sequence together with NGG PAM sequence (N20NGG) was searched on both strands of the target integration site sequence and blasted against the EnN genome. The unique 20-bp sequences were selected as sgRNA of the target integration sites. A 300-500 bp sequence upstream and downstream of the sgRNA were selected as the left homologous arm (LHA) and right homologous arm (RHA). 5.2 sgRNA plasmid construction

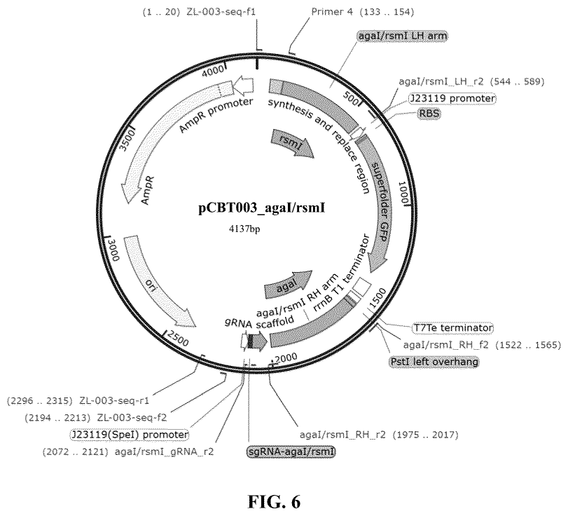

The sgRNA sequence was added to the 5′ end of the reverse primer of the gRNA scaffold on the pCBT003 plasmid, then the gRNA scaffold together with the designed sgRNA sequence was amplified from pCBT003, both the PCR product and the pCBT003 plasmids were digested with PstI and SpeI, the digested pCBT003 plasmid was dephosphorylated, and then ligated with the digested PCR fragments to generate pCBT003_sgRNA plasmid (FIG. 6).

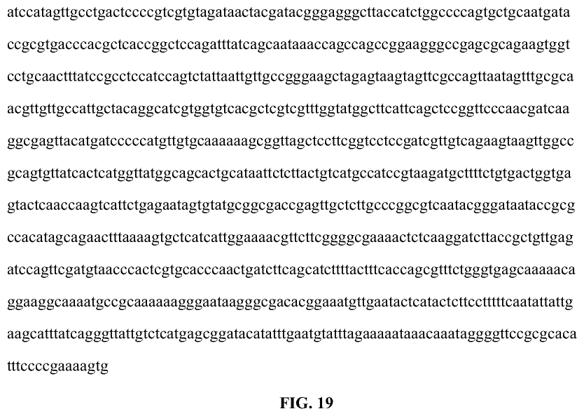

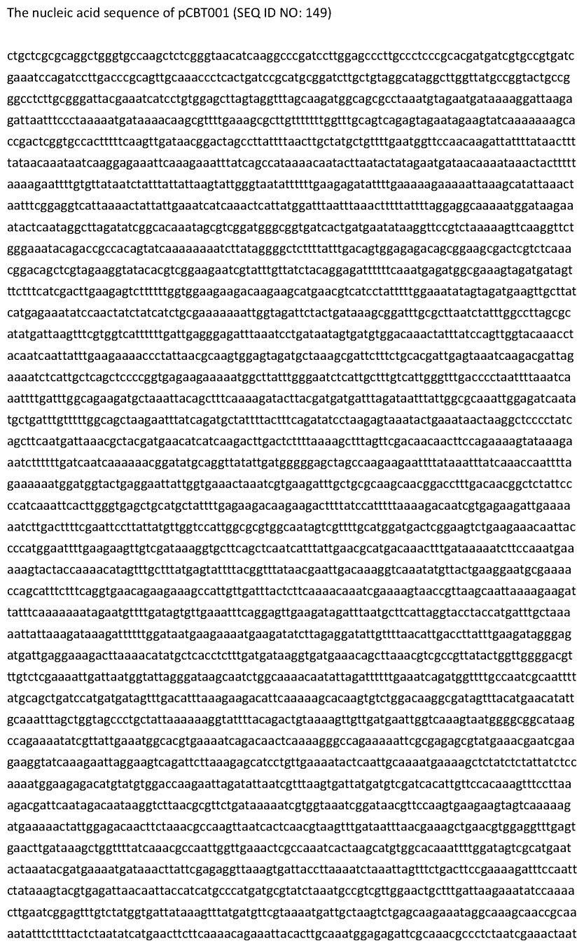

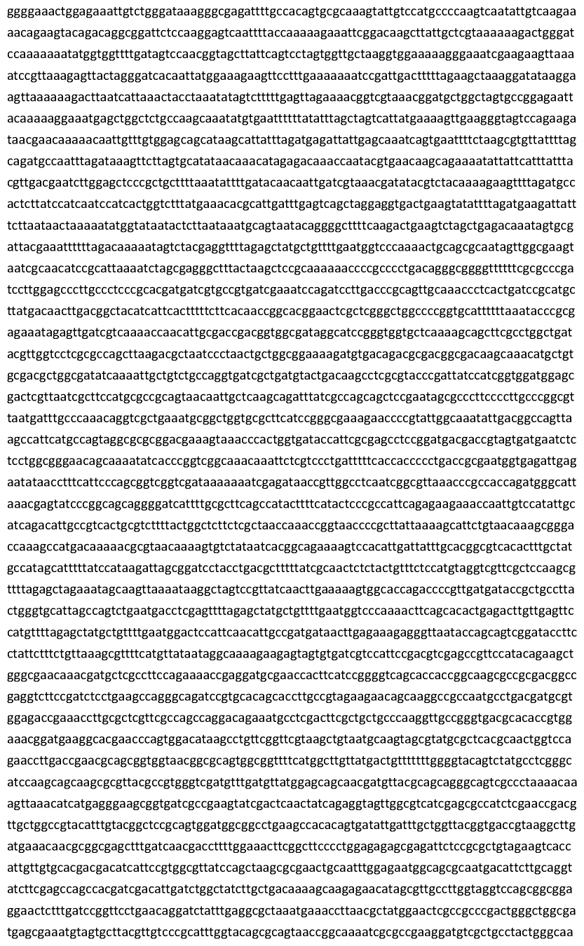

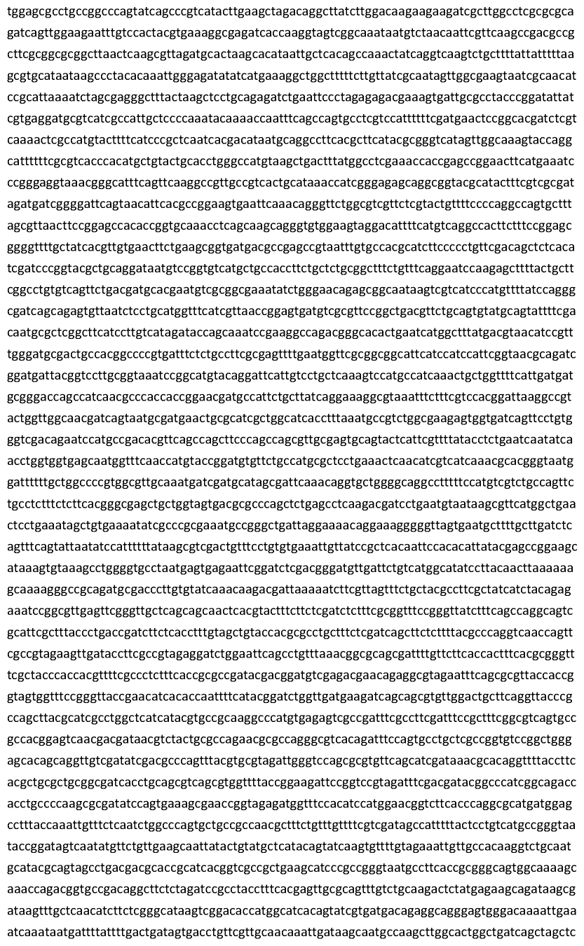

The nucleic acid sequence of pCBT003 is set forth in SEQ ID NO: 150 shown in FIG. 19.

5.3 Donor Gene Cassette Design

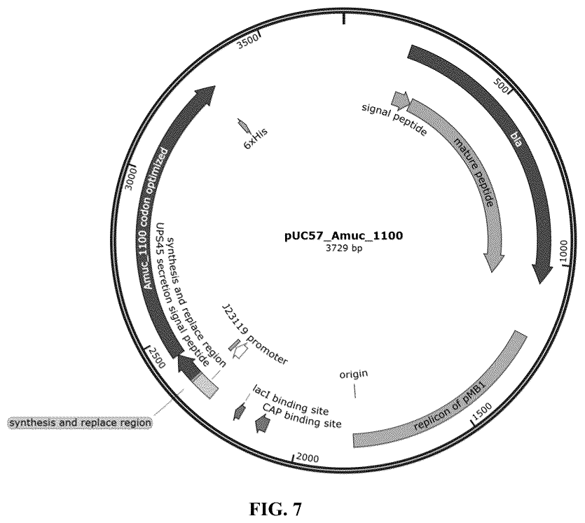

The target gene to be integrated to the genome of EcN was synthesized on a cloning plasmid (e.g. pUC57) by GeneScript (pUC57_Amuc_1100, as shown in FIG. 7). The target gene was amplified from plasmid pUC57_Amuc_1100, LHA and RHA of the selected integration sites were amplified from the genome of EcN. The primers used for PCR of these fragments had 15-20 bp homologous sequence with each other, so that they can be ligated by overlap PCR with the target gene flanked by LHA and RHA. The linear PCR product was used as donor gene cassette.

5.4 Plasmid Electroporation

5.4.1 Making Electrocompetent Cells of EcN

Single colony of EcN on LB agar plates was inoculated to 3 mL LB media in test tube and incubated at 37° C. overnight with shaking (225 rpm), 300 μL of this overnight cultures was inoculated to 30 mL LB media in 125 flasks, incubated at 37° C. with shaking (225 rpm) until OD₆₀₀ reached ˜0.6, then the cells are washed 3 times with 20 mL, 10 mL, and 5 mL 10% ice cold glycerol and final resuspended in 300 μL 10% ice cold glycerol and separated to 1.5 mL centrifuge tubes with 100 μL in each tube.

5.4.2 Electroporation of pCBT001 plasmid

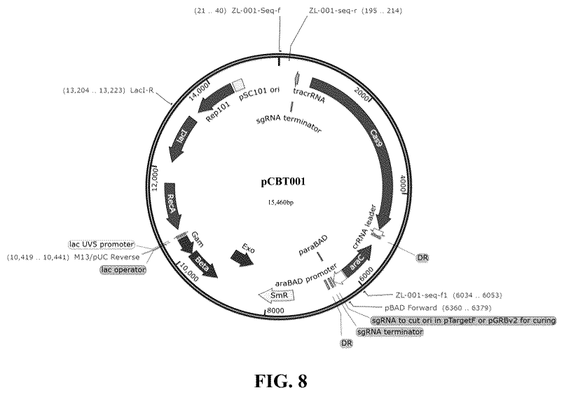

100-200 ng pCBT001 plasmid (i.e. a plasmid expressing Cas9 protein, see polynucleotide sequence in SEQ ID NO: 149) was added to 100 μL EcN electrocompetent cells, the mixture was transferred to a 2 mm electroporation cuvette and electroporated at 2.5 kV, 25 μF, 200Ω. The cells were then recovered by resuspending in 1 mL SOC and incubated at 30° C. with shaking (225 rpm) for two hours. After recovery, all cells were plated on LB agar plates supplemented with 50 μg/mL spectinomycin and streptomycin and incubated at 30° C. overnight. Colonies grew on the plates were EcN harboring pCBT001 plasmid (EcN/pCBT001, as shown in FIG. 8).

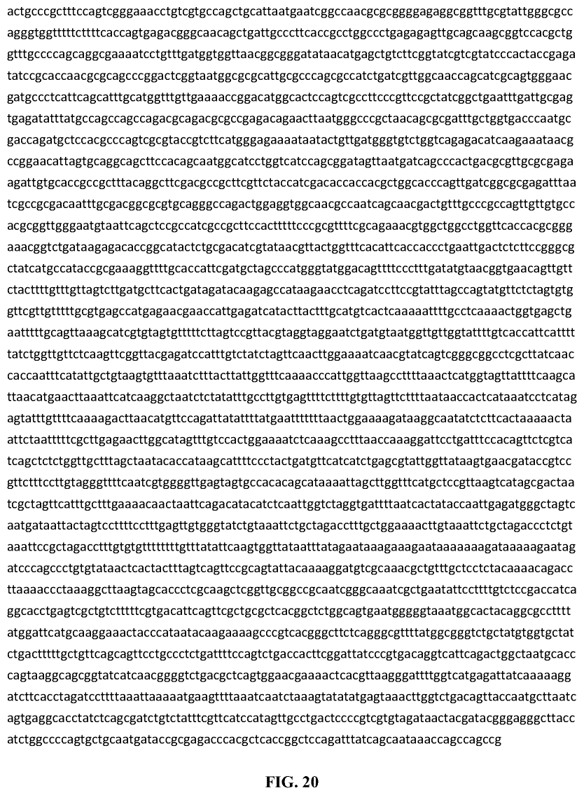

The nucleic acid sequence of pCBT001 is set forth in SEQ ID NO: 149 shown in FIG. 20.

5.4.3 Making electrocompetent cells of EcN/pCBT001

Single colony of EcN/pCBT001 on LB agar plates supplemented with 50 μg/mL spectinomycin and streptomycin was inoculated to 3 mL LB liquid media supplemented with 50 μg/mL spectinomycin and streptomycin in test tube and incubated at 30° C. overnight with shaking (225 rpm), 300 μL of this overnight cultures was inoculated to 30 mL LB liquid media supplemented with 50 μg/mL spectinomycin and streptomycin in 125 flasks, incubated at 30° C. with shaking (225 rpm). After 1 hours incubation, IPTG was added to the culture with a concentration of 1 mM. When OD₆₀₀ reached ˜0.6, the cells were washed 3 times with 20 mL, l0 mL, and 5 mL 10% ice cold glycerol and final resuspended in 300 μL 10% ice cold glycerol and separated to 1.5 mL centrifuge tubes with 100 μL in each tube. 5.4.4 Electroporation of pCBT003_sgRNA plasmid and donor gene cassette

˜2 μg PCR product of the donor cassette contained the target gene flanked by LHA and RHA of the integration site and 100-200ng pCBT003_sgRNA expressing the sgRNA of the integration site are transformed into the electrocompetent cells of EcN/pCBT001 as described in section 5.4.2. After recovery in 1 mL SOC at 30° C. for 2 hours, all the cells were plated LB agar plates supplemented with 50 μg/mL spectinomycin, streptomycin and 100 μg/mL ampicillin.

5.5 Verification of target gene integration

Primers for the verification were designed upstream of LHA and downstream of the RHA. Single colonies grew on the plates of section 5.4.4 were picked, and the right clone with the target gene integration were selected based on the size of PCR products using the verification primer.

5.6 Removal of pCBT003_sgRNA Plasmid

The right clones selected were cultured at 30° C. with shaking (220 rpm) overnight in LB liquid media supplemented with 50 μg/mL spectinomycin, streptomycin and 10 mM arabinose, then the cultures were diluted 106 times and plated 100 μL on LB agar plates supplemented with 50 μg/mL spectinomycin and streptomycin. Single colonies were then picked and spotted on both LB agar plates supplemented with 50 μg/mL spectinomycin, streptomycin and 100 μg/mL ampicillin, and LB agar plates supplemented with 50 μg/mL spectinomycin and streptomycin. The colonies only grew on LB agar plates supplemented with 50 μg/mL spectinomycin and streptomycin were the ones have pCBT003_sgRNA plasmid cured.

5.7 Removal of pCBT001 Plasmid

Clones having pCBT003_sgRNA cured from section 5.6 were incubated in LB liquid media at 42° C. overnight, then the cultures were diluted 10⁶ times and plated 100 μL on LB agar plates. Single colonies were then picked and spotted on both LB agar plates and LB agar plates supplemented with 50 μg/mL spectinomycin and streptomycin. The colonies only grew on LB agar plates were the ones having pCBT001 cured.

5.8 Membrane Protein Knock Out Strategies

The design of sgRNA for knocking out target membrane protein and the construction of sgRNA plasmids have been described above. A 300-500 bp sequence upstream and downstream of the target membrane protein coding gene was selected as the left homologous arm (LHA) and right homologous arm (RHA). The LHA and RHA of the selected knockout sites were amplified from the genome of EcN. The PCR primers used for amplifying these fragments had 15-20 bp homologous sequence with each other, so that they can be ligated by overlap PCR, and the obtained PCR product comprising LHA-RHA was used as donor gene. cassette. The obtained PCR product of the donor fragment and the pCBT003-sgRNA plasmid expressing knockout site sgRNA are transformed into the electrocompetent cells simultaneously. The obtained single colonies were amplified by verification primers of knockout sites, and clones having target proteins successfully deleted from the genome were screened based the size of amplification bands.

The knocked out membrane proteins in the examples include tolQ, tolR, tolA, pal, 1pp, mrcA and ompT. The sgRNA sequence of the tolQ knockout site is set forth in SEQ ID NO: 166, and the sequences of the homologous arms flanking the tolQ knockout site are respectively set forth in SEQ ID NO: 167 and SEQ ID NO: 168; the sgRNA sequence of the tolR knockout site is set forth in SEQ ID NO: 169, and the sequences of the homologous arms flanking the tolR knockout site are respectively set forth in SEQ ID NO: 170 and SEQ ID NO: 171; the sgRNA sequence of the tolA knockout site is set forth in SEQ ID NO: 172, and the sequences of the homologous arms flanking the tolA knockout site are respectively set forth in SEQ ID NO: 173 and SEQ ID NO: 174; the sgRNA sequence of the pal knockout site is set forth in SEQ ID NO: 175, and the sequences of the homologous arms flanking the pal knockout site are respectively set forth in SEQ ID NO: 176 and SEQ ID NO: 177; the sgRNA sequence of the 1pp knockout site is set forth in SEQ ID NO: 178, and the sequences of the homologous arms flanking the 1pp knockout site are respectively set forth in SEQ ID NO: 179 and SEQ ID NO: 180; the sgRNA sequence of the mrcA knockout site is set forth in SEQ ID NO: 181, and the sequences of the homologous arms flanking the mrcA knockout site are respectively set forth in SEQ ID NO: 182 and SEQ ID NO: 183; the sgRNA sequence of the ompT knockout site is set forth in SEQ ID NO: 184, and the sequences of the homologous arms flanking the ompT knockout site are respectively set forth in SEQ ID NO: 185 and SEQ ID NO: 186.

The sgRNA sequence targeting the tolQ site(SEQ ID NO: 166):

gggaaacgggataatctgag

The left homologous arm (LHA) sequence of the tolQ site (SEQ ID NO: 167):

gtgaatacaacgctgtttcgatggccggttcgtgtctactatgaagat
accgatgccggtggtgtggtgtaccacgccagttacgtcgctttttat
gaaagagcacgcacagagatgctgcgtcatcatcatttcagtcaacag
gcgctgatggctgaacgcgttgcctttgtggtacgtaaaatgacggtg
gaatattacgcacctgcgcggctcgacgatatgctcgaaatacagact
gaaataacatcaatgcgtggcacctctttggttttcacgcaacgtatt
gtcaacgccgagaatactttgttgaatgaagcagaggttctggttgtt
tgcgttgacccactcaaaatgaagcctcgtgcgcttcccaagtctatt
gtcgcggagtttaagcagtga

The right homologous arm (RHA) sequence of the tolQ site (SEQ ID NO: 168):

gccatggccagagcgcgtggacgaggtcgtcgcgatctcaagtccgaa
atcaacattgtaccgttgctggacgtactgctggtgctgttgctgatc
tttatggcgacagcgcccatcatcacccagagcgtggaggtcgatctg
ccagacgctactgaatcacaggcggtgagcagtaacgataatccgcca
gtgattgttgaagtgtctggtattggtcagtacaccgtggtggttgag
aaagatcgtctggagcgtttaccaccagagcaggtggtggcggaagtg
tccagccgtttcaaggccaacccgaaaacggtctttctgatcggtggc
gcaaaagatgtgccttacgatgaaataattaaagcactgaacttgtta
catagtgcgggtgtgaaatcggttggtttaatgacgcagcctatctaa
acatctgcgtttcccttgcttgaaagagagcgggtaacaggcgaacag
tttttggaaaccgaga

The sgRNA sequence targeting the tolR site (SEQ ID NO: 169):

tggtattggtcagtacaccg

The left homologous arm (LHA) sequence of the tolR site (SEQ ID NO: 170):

gcatcgtgccaatagccatgcgccggaagccgtagtggaaggggcgtcg
cgtgcgatgcgtatttccatgaatcgtgaacttgaaaatctggaaacgc
acattcctttcctcggtacggttggctccatcagcccgtatattggtct
gtttggtacggtctgggggatcatgcacgcctttatcgccctcgggggg
taaaacaagcaacactgcaaatggttgcgcccggtatcgcagaagcgtt
gattgcgacggcaattggtttgttcgccgcaattccggcggtaatggct
tataaccgcctcaaccagcgcgtaaacaaactggaactgaattacgaca
actttatggaagagtttaccgcgattctgcaccgccaggcgtttaccgt
tagcgagagcaacaaggggtaa

The right homologous arm (RHA) sequence of the tolR site (SEQ ID NO: 171):

acatctgcgtttcccttgcttgaaagagagcgggtaacaggcgaacagt
ttttggaaaccgagagtgtcaaaggcaaccgaacaaaacgacaagctca
agcgggcgataattatttcagcagtgctgcatgtcatcttatttgcggc
gctgatctggagttcgttcgatgagaatatagaagcttcagccggaggc
ggcggtggttcgtccatcgacgctgtcatggttgattcaggtgcggtag
ttgaacagtacaaacgtatgcaaagccaggaatcaagcgcgaagcgttc
tgatgagcagcgcaagatgaaggagcagcaggctgctgaagaactgcgt
gagaaacaagcggctgaacaggaacgcctgaagcaacttgagaaagagc
ggttagcggctcaggagcagaaaaagcaggctgaagaagccgcaaaaca
ggccgagttaaagcagaagcaagcggaagaggc

The sgRNA sequence targeting the tolA site (SEQ ID NO: 172):

agaactgcgtgagaaacaag

The left homologous arm (LHA) sequence of the tolA site (SEQ ID NO: 173):

gccatggccagagcgcgtggacgaggtcgtcgcgatctcaagtccgaaa
tcaacattgtaccgttgctggacgtactgctggtgctgttgctgatctt
tatggcgacagcgcccatcatcacccagagcgtggaggtcgatctgcca
gacgctactgaatcacaggcggtgagcagtaacgataatccgccagtga
ttgttgaagtgtctggtattggtcagtacaccgtggtggttgagaaaga
tcgtctggagcgtttaccaccagagcaggtggtggcggaagtgtccagc
cgtttcaaggccaacccgaaaacggtctttctgatcggtggcgcaaaag
atgtgccttacgatgaaataattaaagcactgaacttgttacatagtgc
gggtgtgaaatcggttggtttaatgacgcagcctatctaaacatctgcg
tttcccttgcttgaaagagagcgggtaacaggcgaacagtttttggaaa
ccgaga

The right homologous arm (RHA) sequence of the tolA site (SEQ ID NO: 174):

tcgcgatgttgactgttccgacggtcaacatcaggcaccggttgccacg
gggttctggtagttttgtgtattttagtttgttaacattctgctaaatt
atcgtgggccatcggtccagataagggagatatgatgaagcaggcatta
cgagtagcatttggttttctcatactgtgggcatcagttctgcatgctg
aagtccgcattgtgatcgacagcggtgtagattccggtcgtcctattgg
tgttgttcctttccagtgggcggggcctggtgcggcacctgaagatatt
ggcggcatcgttgctgctgacttgcgtaacagcggtaaatttaatccgt
tagatcgtgctcgtttgccacagcagccgggtagtgcgcaggaagtaca
accagctgcatggtccgcgctgggcattgacgctgtggttgtcggtcag
gtcactccgaatccggatggttcttacaatgttgcttatcaacttgttg
acactggcgg

The sgRNA sequence targeting the pal site (SEQ ID NO: 175):

agtcaccgtagaaggtcacg

The left homologous arm (LHA) sequence of the pal site (SEQ ID NO: 176):

tggtcgcagtaacaataccgaaccgacctggttcccggacagccagaacctggcatttacttctgaccaggccggtcgtccgcaggt
ttataaagtgaatatcaacggcggtgcgccacaacgtattacctgggaaggttcgcagaaccaggatgcggatgtcagcagcgacg
gtaaatttatggtaatggtcagctccaacggtgggcagcagcacattgccaaacaagatctggcaacgggaggcgtacaagttctgt
cgtccacgttcctggatgaaacgccaagtctggcacctaacggcactatggtaatctacagctcttctcaggggatgggatccgtgct
gaatttggtttctacagatgggcgtttcaaagcgcgtcttccggcaactgatggacaggtcaaattccctgcctggtcgccgtatctgtg
ataataattaattgaatagtaaaggaatcattgaa

The right homologous arm (RHA) sequence of the pal site (SEQ ID NO: 177):

gagaattgcatgagcagtaacttcagacatcaactattgagtctgtcgttactggttggtatagcggccccctgggccgcttttgctcag
gcaccaatcagtagtgtcggctcaggctcggtcgaagaccgcgtcactcaacttgagcgtatttctaatgctcacagccagcttttaac
ccaactccagcaacaactctctgataatcaatccgatattgattccctgcgtggtcagattcaggaaaatcagtatcaactgaatcaggt
cgtggagcggcaaaagcagatcctgttgcagatcgacagcctcagcagcggtggtgcagcggcgcaatcaaccagcggcgatca
aagcggtgtggcggcatcaacgacgccgacagctgatgctggtactgcgaatgctggcgcgccggtgaaaagcggtgatgcaaa
cacggattacaatgcagctattgcgctggtgcag

The sgRNA sequence targeting the 1pp site (SEQ ID NO: 178):

acgttcaggctgctaaagat

The left homologous arm (LHA) sequence of the 1pp site (SEQ ID NO: 179):

aatacttcccggatgccaccatcctggcactgaccaccaacgaaaaaacggctcaccagttagtactgagcaaaggtgttgtgccgc
agctggttaaagagatcacatctactgatgacttctaccgtctgggtaaagaactggctctgcagagcggtctggcacacaaaggtga
tgtcgtagttatggtttctggtgcactggtaccgagcggcactactaacaccgcatctgttcacgtcctgtaatattgcttttgtgaattaat
ttgtatatcgaagcgccctgatgggcgctttttttatttaatcgataaccagaagcaataaaaaatcaaatcggatttcactatataatctca
ctttatctaagatgaatccgatggaagcatcctgttttctctcaatttttttatctaaaacccagcgttcgatgcttctttgagcgaacgatca
aaaataagtgccttcccatcaaaaaaa

The right homologous arm (RHA) sequence of the 1pp site (SEQ ID NO: 180):

cctgtgaagtgaaaaatggcgcacattgtgcgccatttttttgcctgctatttaccgctactgcgtcgcgcgtaacatattcccttgctctg
gttccccattctgcgctgactctactgaaggcgcattgctggctgcgggagttgctccactgctcaccgcaaccggataccctgcccg
acgatacaacgctttatcgactaacttctgatctacagccttattgtctttaaattgcgtaaagcctgctggcagcgtgtacggcattgtct
gaacgttctgctgttcttctgccgatagtggtcgatgtacttcaacataacgcatcccgttaggttccacggaatatttcaccggttcgttg
atcactttcaccggtgttcccgtccgcacgctggagaataaagctttaatatccggtgcattcatgcgaataca

The sgRNA sequence targeting the mrcA site (SEQ ID NO: 181):

caaaccgttcctctacaccg

The left homologous arm (LHA) sequence of the mrcA site (SEQ ID NO: 182):

gacagccaggccatttgctcccgctcaccgagggacatcgacgggcgtggaaatgaccgctgtaatgtgcgactggcgggaaacg
ccaacataatgtgatgacgctgcggcagttcacgactccacggtaacaacgtgttagccagccgctgcacatcaacaatccgcccat
ctttgataatgtcgttctccagtggcaaccgccaccagcggtgcaataagcattcttttgcgctccgcacgatagcaaccgctaccgctt
cttgctgttgtaaatgcaaaccaatttgccagttcttaaatgccattgtgatgatctccttatcacccgtcactctgacgggtatatcaatgc
gtctggcttgcctttatactaccgcgcgtttgtttataaactgcccaaatgaaactaaatgg

The right homologous arm (RHA) sequence of the mrcA site (SEQ ID NO: 183):

ttaaaaaggcgcttcggcgcctttttcagtttgctgacaaagtgcacttgtttatgccagatgcgggtgaacgcgttatccggccaacaa
aaaattgaaaattcaataaattgcaggaacttcgtaagcctgataagcgttgtgcatcaggcaaacttcacgcatttacactcgcccctg
ccctttcaaccattcgcgcacgaggaacagcgcgctgacattacgcgcttcgttgaagtcaggggcttccagcaaatccatcatatgc
gccagcggccagcgcacctgtggtagcggctctggctcatcgccttccagtgattccgggtagagatcttgcgctaccacgatattca
ttttgctggaaaagtaagacggtgccatgctgagcttcttcaaaaaagtcagatcgttcgcaccaaatccaacctcttcttttagctcgcg
gttagcggcttca

The sgRNA sequence targeting the ompT site (SEQ ID NO: 184):

cctgggactcatggccggat

The left homologous arm (LHA) sequence of the ompT site (SEQ ID NO: 185):

cccagcccttgtcgatgccgcgttcaggctagcttcgttcaaatccattaaagatatcagactactctcaggtacgtgagtaaggtaaaa
cccagttccaacgccaatatccagatggttgttacctaaatgttccagaaagtgtggaagaaggtgttcctttgtaggacatccccatgc
aagccgatttgatactcccaaaacccaccagtcataaagctttagggtaagtggtgtgtaaattttagccccatcatctgtgttttttttcatt
agtttcaccgtgttatagttttatttgtgaattaaatcaattatagagatgaattacaaggggttaaatgatgccacggcataacgaatttga
atatattgggtcttgtgttgcggtaagaggttacacgccaggacgcaggttaaatgcttacaatattaggggatattgtttgcttttaacgg
taaacaaattccccggggctatacaataccaccggggagaaaatctatttaacgttg

The right homologous arm (RHA) sequence of the ompT site (SEQ ID NO: 186):

aaaaggtctccattcaatcgttttaatgattgagtatgtattttttatatctaacttaatgagtcaattgtatattgccccactgtttatattt
tgtttaacattgaatttttgtcacaatgcgctgtcagttttttagtttaattgtctgtttgtgttttgtattgtggttttatgggctaatta
tttttttctttgtggagtttttatctattcaagtgccatgcctttagatgcatattagcgataatagcatgaggtttatcctcaattgctgtg
ttttttagtataaaaaagaggaacaaaactgagacacataaggcctcgcaatggcttgcaaggttttacatattttgaggtggtggaacgtgt
gaacgcagagatggcgcggtaatttgttgatttaaaatgtcgttcttggagtttctaagtcgtgggctacaggttcgaatcctgcagggcgag
ccatttcctcgccatttatttgttcccttttacaaattacaccatcactttgttgtcgcggttttgctaaataattggaacacgtcagacgaa
taaaatgaaaacagaaattctgctaaacaattcatcttgccaggattttgattaggaaagtgaaa

### Example 6. Production of Genetically Engineered Bacteria Encoding and Secreting Amuc_1100 (31-317)

6.1 Construction of Amuc_1100 Expression Cassette

(1) PCR of the Amuc_1100 Expression Cassettes

The Amuc_1100 encoding sequence, signal peptide, promoter, RBS, cistron and such were synthesized on a cloning plasmid pUC57 by GeneScript. The Amuc_1100 encoding sequence, signal peptide, promoter, RBS, cistron and such were amplified with the synthesized plasmid as a template. (pUC57_Amuc_1100, as shown in FIG. 7). The LHA and RHA of the integration sites were amplified from the genome of EcN. The transcription terminator was amplified with plasmid pCBT003 as a template. The PCR primers used for amplifying these fragments had 15-20 bp homologous sequence with each other, so that they can be ligated by overlapping PCR, to obtain the donor gene fragment cassette flanked by LHA and RHA. The elements of the cassette were arranged from 5′ to 3′ as follows: 5′-promoter-RBS-cistron-signal peptide-Amuc_1100 encoding sequence-terminator. The Amuc_1100 encoding sequence is wild-type Amuc_1100 encoding sequence (SEQ ID NO: 1, denoted as “Wildtype” in table 5) or Amuc_1100 Y289A mutant (the numbering of the position 289 is relative to SEQ ID NO: 1, denoted as Y289A in table 5).

PCR reaction was performed to amplify the target gene, and then the electro-competent cells were prepared according to the methods similar to those described in section 5.4.1. Then, electroporation of the electro-competent cells was performed according to the methods similar to those described in section 5.4.2. Recovery after electroporation was conducted by the procedures below. 1 mL SOC was added to the cells immediately after electroporation, and the cells were incubated at 30° C., 220 rpm for 1-2 hours, then all the cells were plated to LB agar plates with antibiotics, incubated at 30° C. overnight.

(2) Genomic Integration of Amuc_1100 Expression Cassettes

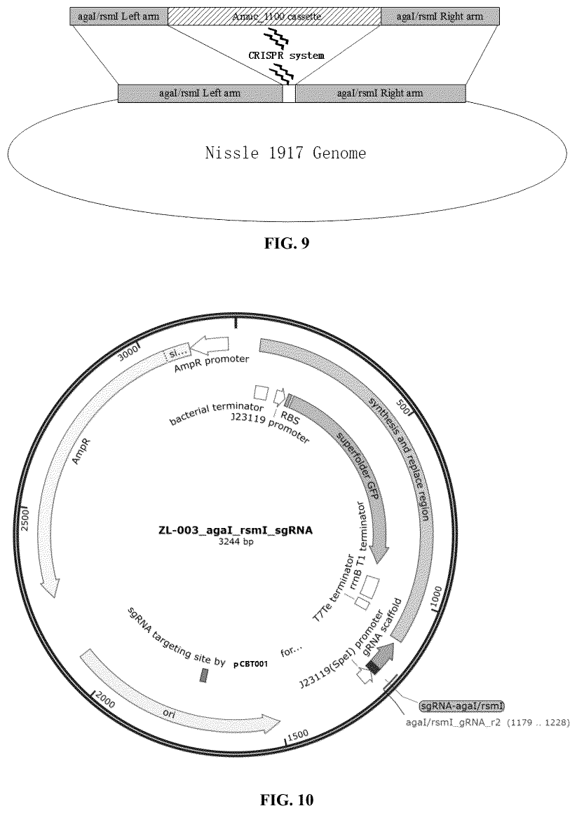

The PCR fragments of Amuc_1100 expression cassette was integrated to selected sites of EcN using CRISPR-Cas9 (FIG. 9). Briefly, the Amuc_1100 expression cassette PCR fragments and the plasmid pCBT003_agaI/rsmI_sgRNA, pCBT003_malP/T-sgRNA, pCBT003_pflB-sgRNA or pCBT003_lldD-sgRNA (a representative, pCBT003_agaI/rsmI_sgRNA, is shown in FIG. 10, referred to as ZL-003_agaI/rsmI_sgRNA) were electroporated to the EcN strain containing pCBT001 (ZL-001). The successful integration of respective Amuc_1100 expression cassette was verified by PCR.

To obtain Amuc_100 expressing strains with multiple copies, two, three or more cassette were incorporated into EcN genome; when one copy of Amuc_1100 was inserted, the integration site is the agaI/rsmI site of the EcN genome; when two copies of Amuc_1100 were inserted, the first and second integration sites were the agaI/rsmI site and malP/T site of the EcN genome; when three copies of Amuc_1100 were inserted, the first, second and third integration sites were the agaI/rsmI site, malP/T site and pflB site.

The sgRNA sequence targeting the agaI/rsmI site is set forth in SEQ ID NO: 200, and the sequences of the flanking homologous arms of the agaI/rsmI site are set forth in SEQ ID NO: 201 and 202 respectively. The sgRNA sequence targeting the malP/T site is set forth in SEQ ID NO: 203, and the sequences of the flanking homologous arms of the malP/T site are set forth in SEQ ID NO: 204 and 205 respectively. The sgRNA sequence targeting the pflB site is set forth in SEQ ID NO: 206, and the sequences of the flanking homologous arms of the pflB site are set forth in SEQ ID NO: 207and 208 respectively. The sgRNA sequence targeting the lldD site is set forth in SEQ ID NO: 209, and the sequences of the flanking homologous arms of the malP/T site are set forth in SEQ ID NO: 210 and 211 respectively. The sgRNA sequence targeting the maeA site is set forth in SEQ ID NO: 212, and the sequences of the flanking homologous arms of the maeA site are set forth in SEQ ID NO: 213 and 214 respectively.

The sgRNA sequence targeting the agaI/rsmI site (SEQ ID NO: 200):

tgtgtcgtcgttaactcgcc

The left homologous arm (LHA) sequence of the agaI/rsmI site (SEQ ID NO: 201):

gccgcgcacgctgattttttatgaatctacccaccgtctgttagatagcctggaagatatcgttgcggtattaggcgaatcccgttacgtg
gttctggcgcgtgagctaaccaaaacctgggaaaccattcacggcgcgcccgttggcgagctgctggcgtgggtaaaagaagatg
aaaaccgtcgcaaaggcgaaatggtgctgattgtcgaaggccataaagcgcaggaaaatgacttgcctgctgatgccctgcgcacg
ctggcgctgctacaggcagaactgccgctgaaaaaagcggcggcgctggccgcagaaattcacggcgtgaagaaaaatgcgctg
tataagtatgcgctggagcagcagggagagtgattacaggcgtcgcagcaggaatagcgctac

The right homologous arm (RHA) sequence of the agaI/rsmI site (SEQ ID NO: 202):

tcacgcatgtgaaatggttatgcagccataaaaatgaagccgggatagcggtggagactttagccgtgagaaaacgttctgtcgcatc
ctgttttccttcgccagtcaccagtaacaaaacttcgcgggcattgagaatatccttcaggcctaaggtgatcccacgagtcacgggac
ggtccgcggtttttaacatctcatgttgctgtgttctggcatcaagttgactgatatggcaggctggttgcaggctttctcccggttcgttc
agcccaagatgaccgtttttccccaatccgagaacgcataaatccagaccgcctttgcgcgcaatcaggttcgttacccgttcgcactc
tgtctcatttatctcttcggagcgaaagctgatgagctggtcttcac

The sgRNA sequence targeting the malP/I site (SEQ ID NO: 203):

Cgctttttgaaaatacgcaa

The left homologous arm (LHA) sequence of the malP/T site (SEQ ID NO: 204):

accagccgagattcaacaggttgttgcccgtcaggcgaccaatcaaaaac
tccattgagatgtagttaacatgtcgctgattcgccactggcttggcgaa
tggctgagcacgcagcatttcggccagtgcttcgctcactgccagccacc
actggcgaggagtcatttcagccgcagaatttaagccataacgctgccac
tgacgtgaaagcgcttcctgaaattgcttatcgttaaaaataggttgtga
cataggagttccacttttcttagattttcaacacaacgttatcgctagtt
tgccaggctcgatgttgaccttcctcatcctgcgggggattaggcaggga
ggagttgcggggatgagcaaggaaatgtgatctcgaccacttaaagctag
tgcaatccacaggattagcagcaaatcaatgccataccgcgcagaaaatc
tgtatctaagtgcaaaaaatggccgtt

The right homologous arm (RHA) sequence of the malP/T site (SEQ ID NO: 205):

cgtattttcaaaaagcggaaggtaactctataaattaagtaaaggagtga
aacagtttcacaagtaaaatatcaagtgtgctccatctcattcttaatag
atttattaagatcatctttttagatggcactttcatcagaaatgaagaag
aaacccttgcttaaatgaaactgatgaacataaggaaaataccgtacaac
ccagtattcacgctggatcagcgtcgttttaggtgagttgttaataaaca
tttggaattgtgacacagtgcaaattcagacacataaaaaaacgtcatcg
cttgcattagaaaggtttctggccgaccttataaccattaattacgaagc
gcaaaaaaaataatatttcctcattttccacagtgaagtgattaactatg
ctgattccgtcaaaattaagtcgtccggttcgactcgaccataccgtggt
tcgtga

The sgRNA sequence targeting the pflB site (SEQ ID NO: 206):

ccgtgacgttatccgcacca

The left homologous arm (LHA) sequence of the pflB site (SEQ ID NO: 207):

acgatgacctgatgcgtccggacttcaacaacgatgactacgctattgct
tgctgcgtaagcccgatgatcgttggtaaacaaatgcagttcttcggtgc
gcgtgcaaacctggcgaaaaccatgctgtacgcaatcaacggcggcgttg
acgaaaaactgaaaatgcaggttggtccgaagtctgaaccgatcaaaggc
gatgtcctgaactatgatgaagtgatggagcgcatggatcacttcatgga
ctggctggctaaacagtacatcactgcactgaacatcatccactacatgc
acgacaagtacagctacgaagcctctctgatggcgctgcacgaccgtgac
gttatcc

The right homologous arm (RHA) sequence of the pflB site (SEQ ID NO: 208):

gtgacgctatcccgactcagtctgttctgaccatcacttctaacgttgtg
tatggtaagaaaactggtaacaccccagacggtcgtcgtgctggcgcgcc
gttcggaccgggtgctaacccgatgcacggtcgtgaccagaaaggtgctg
tagcgtctctgacttccgttgctaaactgccgtttgcttacgctaaagat
ggtatctcctacaccttctctatcgttccgaacgcactgggtaaagacga
cgaagttcgtaagaccaacctggctggtctgatggatggttacttccacc
acgaagcatccatcgaaggtggtcagcacctgaacgttaacgtgatgaac
cgtgaaatgctgctcgacgcgatggaaaacccggaaaaatatccgcagct
gaccatccgtgtatctggctacgcagtacgtttcaactcgct

The sgRNA sequence targeting the lldD site (SEQ ID NO: 209):

gttatcgtgatgcgcattct

The left homologous arm (LHA) sequence of the lldD site (SEQ ID NO: 210):

ttccgcagccagcgattatcgcgccgcagcgcaacgcattctgccgccgt
tcctgttccactatatggatgggggggcatattctgaatacacgctgcgc
cgcaacgtggaagatttgtcagaagtggcgctgcgccagcgtattctgaa
aaacatgtctgacttaagcctggaaacgacgctgtttaatgagaaattgt
cgatgccggtggcgctaggtccggtaggtttgtgtggcatgtatgcgcga
cgcggcgaagttcaggctgccaaagcagcagatgcgcatggcattccgtt
tactctctcgacggtttccgtttgcccgattgaagaagtggctccggcta
tcaaacgtccgatgtggttccagctttatgtgctgcgcgatcgcggcttt
atgcgtaacgccctggagcgagcaaaagccgcgggttgttcgacgctggt
tttcacc

The right homologous arm (RHA) sequence of the lldD site (SEQ ID NO: 211):

atccgcgatttctgggatggcccgatggtgatcaaagggatcctcgatcc
ggaagatgcgcgcgatgcagtacgttttggtgctgatggaattgtggttt
ctaaccacggtggccgccagttagatggcgtactctcttctgctcgtgca
ctgcctgctattgcggatgcggtgaaaggtgatatcgccattctggcgga
tagcggaatacgtaacgggcttgatgtcgtgcgtatgattgcgctcggtg
ccgacaccgtactgctgggtcgtgctttcctgtatgcactggcaacagcg
ggccaggcgggtgtagctaatctgctaaatctgatcgaaaaagagatgaa
agtggcgatgacgctgactggcgcgaaatcgattagcgaaattacgcaag
attcgctggtgcaggggctgggtaaagagttgcctgcggca

The sgRNA sequence targeting the maeA site (SEQ ID NO: 212):

taacacccagcccgatgccc

The left homologous arm (LHA) sequence of the maeA site (SEQ ID NO: 213):

ttctgcatagcaggtgaggcaaatgagatttattcgccactacccagtat
ggatgagatctgaaaaagggagaggaaaattgcccggtagccttcactac
cgggcgcaggcttagatggaggtacggcggtagtcgcggtattcggcgtg
ccagaaattatcatcaatggcctgttgcagagcttcggcagacgttttca
ccgccacgccttgctgctgcgccattttgccaaccgcaaaggcaattgcg
cgggagactttctgaatatctttcagttccggcagtaccagaccttcgcc
gttcaggaccagcggcgaatactgagcaagcatttcacttgccgacatca
gcatctc

The right homologous arm (RHA) sequence of the maeA site (SEQ ID NO: 214):

gagattttcgcgcttctgcaccagtttggtctggaaaggcagcaggttcg
gcatcttgtcggtcagcaggccaaagcgatcgaccataaagactttctgc
cgcgccgcttcctcgcttaatccttcgcgctgggtctgggcgatgatcat
ttcggcaatgccgcatcccgctgaacctgcgccaaggaagacgatttttt
tctcgcttaactgaccacctgccgcgcggctggctgcgatcagtgtgccg
actgttaccgccgcggtgccctgaatgtcatcgttaaaagaacaaatttc
attgcgatagcggttaagtaacggcatcgcatttttttgtgcgaagtctt
caaactgcaacagtacgtccggccagcgttgtttcacagcctggataaat
tcatcaacaaattcatagtattcgtcgtcagtgatacgcggattacgcca
gcccatatacagcggatcgttaagcagctgttggttgttggtgccgacat
ccagcaccaccggaagggtataggccggactgatgccgccacag

The genetically engineered strains in the examples were summarized in Table 5

TABLE 5
Amuc_1100 expressing strains

| | | | | | | | | Whether |
|---|---|---|---|---|---|---|---|---|
| | | | | | Amuch_ | | | or not |
| Name of | | | | | 1100 | | | express |
| engineered | | | | Signal | encoding | | Knocked- | chaperon |
| bacteria | Promoter | RBS | Cistron | peptide | sequence | Terminator | out protein | or not |
| CBT1101 | BBa_J23101 | Synthesized | / | usp45 | Y289A | Terminator1 | lpp | No |
| CBT1102 | BBa_J23110 | Synthesized | / | usp45 | Y289A | Terminator1 | lpp | No |
| CBT1103 | BBa_J23110 | Synthesized | / | usp45 | Y289A | Terminator1 | lpp | No |
| | BBa_J23101 | Synthesized | / | usp45 | Y289A | Terminator1 | lpp | No |
| CBT1104 | BBa_J23101 | Synthesized | / | usp45 | Y289A | Terminator1 | lpp | No |
| | BBa_J23101 | Synthesized | / | usp45 | Y289A | Terminator1 | lpp | No |
| | BBa_J23101 | Synthesized | / | usp45 | Y289A | Terminator1 | lpp | No |
| CBT1105 | BBa_J23101 | Synthesized | / | usp45 | Y289A | Terminator1 | lpp | No |
| | BBa_J23101 | Synthesized | / | usp45 | Y289A | Terminator1 | lpp | No |
| | BBa_J23110 | Synthesized | / | usp45 | Y289A | Terminator1 | lpp | No |
| CBT1106 | BBa_J23101 | Synthesized | / | usp45 | Y289A | Terminator1 | lpp | No |
| | BBa_J23110 | Synthesized | / | usp45 | Y289A | Terminator1 | lpp | No |
| | BBa_J23110 | Synthesized | / | usp45 | Y289A | Terminator1 | lpp | No |
| CBT1107 | Pfnrs | Synthesized | / | usp45 | Wildtype | Terminator1 | lpp + ompT | No |
| CBT1108 | Pfnrs | Synthesized | / | usp45 | Wildtype | Terminator1 | lpp + ompT | No |
| | Pfnrs | Synthesized | / | usp45 | Wildtype | Terminator1 | | No |
| CBT1109 | BBa_J23110 | Synthesized | / | usp45 | Wildtype | Terminator1 | lpp | No |
| | BBa_J23110 | Synthesized | / | usp45 | Wildtype | Terminator1 | | No |
| | Pfnrs | Synthesized | / | usp45 | Wildtype | Terminator1 | | No |
| CBT1110 | BBa_J23110 | Synthesized | / | usp45 | Wildtype | Terminator1 | lpp | No |
| | BBa_J23110 | Synthesized | / | usp45 | Wildtype | Terminator1 | | No |
| | Pfnrs | Synthesized | / | usp45 | Wildtype | Terminator1 | | No |
| | Pfnrs | Synthesized | / | usp45 | Wildtype | Terminator1 | | No |
| CBT1111 | Pfnrs | Synthesized | / | usp45 | Wildtype | Terminator1 | lpp + ompT | No |
| | Pfnrs | Synthesized | / | usp45 | Wildtype | Terminator1 | | No |
| | Pfnrs | Synthesized | / | usp45 | Wildtype | Terminator1 | | No |
| CBT1112 | Pfnrs | Synthesized | / | usp45 | Wildtype | Terminator1 | lpp + ompT | No |
| | Pfnrs | Synthesized | / | usp45 | Wildtype | Terminator1 | | No |
| | Pfnrs | Synthesized | / | usp45 | Wildtype | Terminator1 | | No |
| | Pfnrs | Synthesized | / | usp45 | Wildtype | Terminator1 | | No |
| CBT1113 | BBa_J23101 | Synthesized | / | usp45 | Y289A | Terminator1 | | No |
| | BBa_J23110 | Synthesized | / | usp45 | Y289A | Terminator1 | | No |
| | BBa_J23110 | Synthesized | / | usp45 | Y289A | Terminator1 | | No |
| CBT1114 | BBa_J23110 | Synthesized | / | usp45 | Wildtype | Terminator1 | lpp | No |
| | BBa_J23110 | Synthesized | / | usp45 | Wildtype | Terminator1 | lpp | No |
| CBT1115 | BBa_J23110 | Synthesized | / | usp45 | Wildtype | Terminator1 | lpp + ompT | No |
| | BBa_J23110 | Synthesized | / | usp45 | Wildtype | Terminator1 | lpp + ompT | No |
| CBT1116 | BBa_J23110 | Synthesized | / | usp45 | Y289A | Terminator1 | lpp | Yes |
| | BBa_J23101 | Synthesized | / | usp45 | Y289A | Terminator1 | lpp | |
| CBT1117 | BBa_J23110 | Synthesized | / | usp45 | Wildtype | Terminator1 | lpp | No |
| | BBa_J23101 | Synthesized | / | usp45 | | Terminator2 | | |

### 6.2 Optimization of the Amuc_1100 (31-317) Expression Cassette

After being administered to a subject, for example, to intestinal tract of the subject, the engineered bacteria described herein can not only retain the activity of the bacteria, but can also effectively express Amuc 1100, and further effectively secrete the Amuc_1100 protein to the outside of the engineered bacteria. It requires substantive optimization trials in genetic engineering of the chassis bacterial to obtain the engineered bacteria that can express and secrete Amuc_100 protein while not affecting the activity of the bacteria per se. There are few and limited studies and reports on the expression and secretion of therapeutic proteins from engineered bacteria, especially engineered E. coli. Therefore, this example further tested the expression and secretion of Amuc_1100.

6.2.1 Signal peptide

(1) pelB (from Pectobacterium carotovorum), dsbA (from E. coli), ompA (from E. coli), Cel-CD (catalytic domain of cellulase from Bacillus sp.), Amuc1100 (from Akkermansia muciniphila) and usp45 (from Lactococcus lactis) signal peptides were selected to construct expression cassettes that could express and secrete Amuc_1100 protein: the express cassette, Pfnrs-signal peptide-Amuc_100 protein-rrnB_T1_T7Te terminator, was incorporated into the plasmid vector pCBT010 (SEQ ID NO: 151). (2) The constructed plasmid was electroporated into EcNΔlpp. (3) The overnight culture broth was inoculated into 50 mL LB medium at a ratio of 1%, cultured at 37° C. and 220 rpm for 6h, and centrifuged at 7500 rpm to collect the culture supernatant and precipitate. (4) The expression of Amuc_1100 secreted by different signal peptide strains was measured by Western Blot. The Western Blot sample preparation method was: 40 μL supernatant was added to 10μL loading buffer, and the loading volume was 10 μL.

The nucleic acid sequence of pCBT010 (SEQ ID NO: 151):

gaattccggatgagcattcatcaggcgggcaagaatgtgaataaaggccg
gataaaacttgtgcttatttttctttacggtctttaaaaaggccgtaata
tccagctgaacggtctggttataggtacattgagcaactgactgaaatgc
ctcaaaatgttctttacgatgccattgggatatatcaacggtggtatatc
cagtgatttttttctccattttagcttccttagctcctgaaaatctcgat
aactcaaaaaatacgcccggtagtgatcttatttcattatggtgaaagtt
ggaacctcttacgtgccgatcaacgtctcattttcgccaaaagttggccc
agggcttcccggtatcaacagggacaccaggatttatttattctgcgaag
tgatcttccgtcacaggtatttattcgggatcctcgctcactgactcgct
acgctcggtcgttcgactgcggcgagcggaaatggcttacgaacggggcg
gagatttcctggaagatgccaggaagatacttaacagggaagtgagaggg
ccgcggcaaagccgtttttccataggctccgcccccctgacaagcatcac
gaaatctgacgctcaaatcagtggtggcgaaacccgacaggactataaag
ataccaggcgtttccccctggcggctccctcgtgcgctctcctgttcctg
cctttcggtttaccggtgtcattccgctgttatggccgcgtttgtctcat
tccacgcctgacactcagttccgggtaggcagttcgctccaagctggact
gtatgcacgaaccccccgttcagtccgaccgctgcgccttatccggtaac
tatcgtcttgagtccaacccggaaagacatgcaaaagcaccactggcagc
agccactggtaattgatttagaggagttagtcttgaagtcatgcgccggt
taaggctaaactgaaaggacaagttttggtgactgcgctcctccaagcca
gttacctcggttcaaagagttggtagctcagagaaccttcgaaaaaccgc
cctgcaaggcggttttttcgttttcagagcaagagattacgcgcagacca
aaacgatctcaagaagatcatctgtaggtcgttcgctccaagctggaagg
atcttcacctagatccttttaaattaaaaatgaagttttaaatcaatcta
aagtatatatgagtaaacttggtctgacagaatttctgccattcatccgc
ttattatcacttattcaggcgtagcaaccaggcgtttaagggcaccaata
actgccttaaaaaaattacgccccgccctgccactcatcgcagtactgtt
gtaattcattaagcattctgccgacatggaagccatcacaaacggcatga
tgaacctgaatcgccagcggcatcagcaccttgtcgccttgcgtataata
tttgcccatggtgaaaacgggggcgaagaagttgtccatattggccacgt
ttaaatcaaaactggtgaaactcacccagggattggctgagacgaaaaac
atattctcaataaaccctttagggaaataggccaggttttcaccgtaaca
cgccacatcttgcgaatatatgtgtagaaactgccggaaatcgtcgtggt
attcactccagagcgatgaaaacgtttcagtttgctcatggaaaacggtg
taacaagggtgaacactatcccatatcaccagctcaccgtctttcattgc
catacg

The results are shown in FIG. 12B and Table 6. Compared with the original signal peptide of Amuc_1100, the E. coli signal peptides Dsb and ompA, the Cel-CD signal peptide from Bacillus sp. and pelB signal peptide from Pectobacterium carotovorum, the use of Usp45 signal peptide could significantly increase the secretion of Amuc_1100. This result was unexpected as Usp45 signal peptide has been thought to merely work in Lactococcus lactis and the ability to direct secretion of proteins in E. coli has never been contemplated or explored.

To further test the efficiency of different signal peptides in the context of genetically engineered bacteria, other than bacteria transfected with expression vectors as shown above, signal peptides DsbA, OmpA, PelB and YebF were selected, together with Usp45 signal peptide, to construct engineered bacteria whose genome was integrated with expression cassettes carrying different signal peptides and expressing a test protein other than the Amuc_1100 protein. The supernatant and cytoplasmic production of the test protein in the culture after being cultured in LB for 24 hours were shown in Table 6. Among them, the test strain 1 used the dsbA signal peptide; the test strain 2 used the ompA signal peptide; the test strain 3 used the pelB signal peptide; the test strain 4 used the yebF signal peptide, the test strain 5 used the USP45 signal peptide. At the same dilution, the concentrations of the test protein in the engineered bacteria samples using OmpA, PelB, and YebF signal peptides were lower than the detection baseline; the concentration of the test protein in the engineered bacteria samples using DsbA signal peptides was higher than the detection baseline. When the USP45 signal peptide was used, the yield of the test protein was significantly higher than that of the engineered bacteria using DsbA signal peptide.

TABLE 6
Engineered bacteria using different signal peptides
and the production of the test protein

| | Signal peptide in | production of the test protein | |
|---|---|---|---|
| | the expression | (ng/mL) | |
| Strains | cassette | Supernatant | Cytoplasmic |
| test strain 1 | DsbA | 1.02 ± 0.73 | 1.23 ± 0.05 |
| test strain 2 | OmpA | —* | — |
| test strain 3 | PelB | — | — |
| test strain 4 | YebF | — | — |
| test strain 5 | USP45 | 6.15 ± 4.12 | 4.47 ± 3.39 |

*“—” indicates below the detection baseline

The above results indicate that Usp45 signal peptide is the most suitable signal peptide for engineering E. coli to express and secrete exogeneous proteins.

6.2.2 Promoter and Copy Number

First, the effect of the constitutive promoters BBa_J23101 and BBa_J23110 and two-copy combination thereof on the Amuc_1100 (Y289A) protein were tested. For this reason, the strains were constructed as follows: (1) CBT1101, containing a single copy of Amuc_1100 (Y289A) expression cassette driven by the BBA_J23101 promoter; (2) CBT1102, containing a single copy of the Amuc_100 (Y289A) expression cassette driven by the BBA_J23110 promoter; (3) CBT1103, containing two copies of the Amuc_1100 (Y289A) expression cassettes driven by the BBA_J23110 promoter; (4) CBT1104, containing three copies of the Amuc_1100 (Y289A) expression cassettes driven by the BBA_J23101 promoter, (5) CBT1105, containing a single copy of the Amuc_100 (Y289A) expression cassette driven by the BBA_J23101 promoter and two copies of the Amuc_1100 (Y289A) expression cassette driven by the BBA_J23110 promoter; (6) CBT1106, containing three copies of the Amuc_100 (Y289A) expression cassettes driven by the BBA_J23110 promoter.

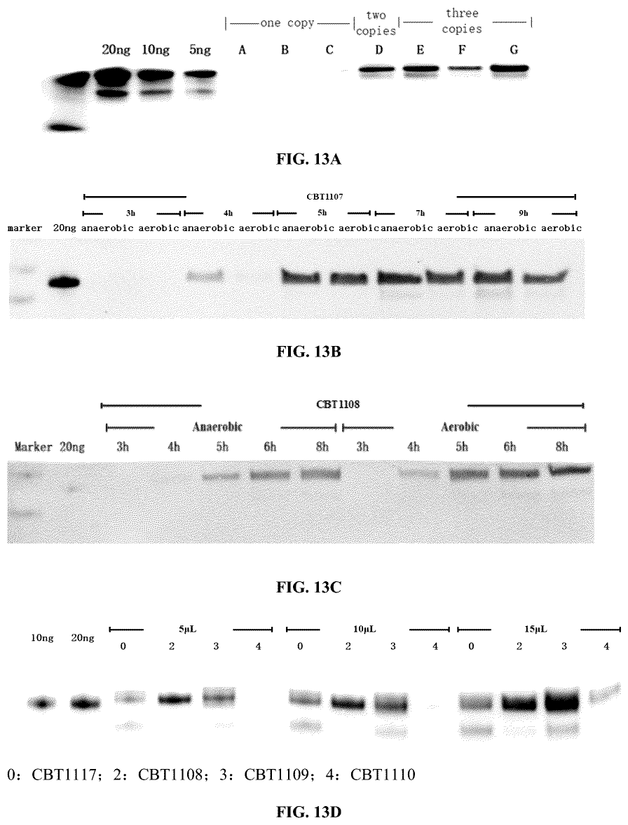

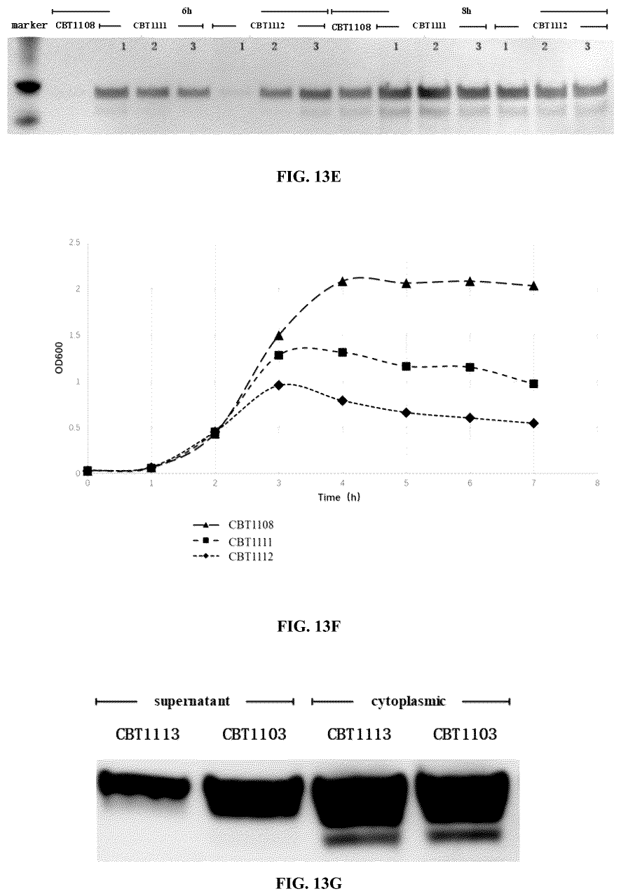

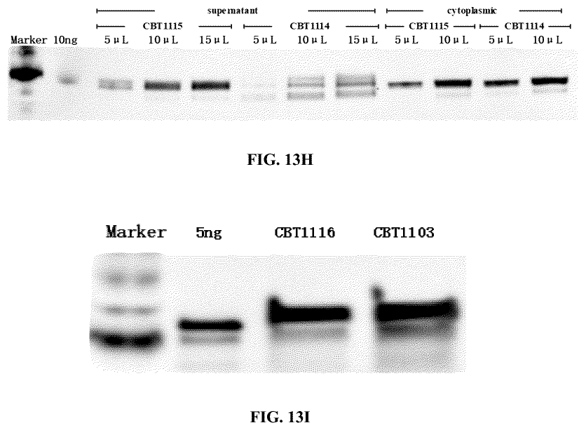

Experimental method: the overnight cultured bacterial solution was inoculated into 50 mL LB medium at a ratio of 1%, cultured at 37° C. 220 rpm for 6 hours, and centrifuged at 12000 rpm to collect the culture supernatant and precipitate. Western Blot (WB) sample processing method: 40 μL of supernatant was taken and added with 10 μL of loading buffer; WB (loading volume of 10 μL) was used to determine the concentration of Amuc_1100, and the result was shown in FIG. 13A, all strains could successfully secrete Amuc_1100 protein, and strains having two copies and three copies of expression cassettes exhibited significantly higher expression level. In addition, the expression intensities of Amuc_1100 when using the two promoters mentioned above were similar, and both can be used for the expression of Amuc_1100 protein. Conclusion: Both the BBA_J23110 promoter and the BBA_J23101 promoter can be used for Amuc_1100 expression; and increasing the copy number of expression cassettes can increase the expression and secretion of Amuc_1100 protein.

Considering that after the engineered bacteria enter the intestinal environment, they will be in a hypoxic environment, so the expression and secretion effects of the anaerobic promoter was further tested, and the strain CBT1107 using a single copy of the anaerobic promoter Pfnrs was constructed and CBT1108 using double copies of the anaerobic promoter Pfnrs was constructed (see Table 5 for specific structure information of the strains).

Experimental method: the overnight cultured bacterial solution was inoculated with 2 bottles of 50 mL medium at a ratio of 1%, and cultured at 37° C. 220 rpm to an OD 0.8. Then transfer one bottle to the anaerobic incubator. Thereafter, 4 bottles of bacterial solution were sampled every 1 or 2 hours, centrifuged at 12000 rpm, and the supernatant and precipitation samples were stored for WB detection of Amuc_1100 concentration. Western Blot sample processing method: 40 μL of supernatant was taken and added with 10 μL of loading buffer; WB (loading volume of 15 μL) was used to determine the concentration of Amuc_1100. The results were shown in FIGS. 13B and 13C. By comparing the protein production (compared with the relative value of the 20ng fluorescence intensity of the standard product) from the CB T 1107 and CBT1108 after 5h aerobic or anaerobic culture, it can be seen that the protein secretion from the CBT1108 strain was about twice that from the CBT1107 strain. This indicated that the anaerobic promoter Pfnrs can be used in E. coli for expressing the Amuc_1100 protein, and that increasing the copy number can improve the expression and secretion effect.

Next, more strains with different promoter and copy number combinations were constructed, and the effects of aerobic fermentation conditions (simulating an in vitro production environment) on the secretion of Amuc_1100 protein were tested. The constructed and tested strains were as follows: CBT1117 (containing two constitutive promoters, namely BBa_J23110 and BBa_J23101), CBT1108 (containing two copies of the Pfnrs promoter), CBT1109 (containing two copies of the BBa_J23110 promoter+single copy of the Pfnrs promoter) and CBT1110 (containing two copies of the BBa_J23110 promoter+two copies of the Pfnrs promoter).

Experimental process: picked single colonies of CBT1117, CBT1108, CBT1109 and CBT1110 into 50 mL LB medium for culture at 37° C. 220 rpm for 7.5h, centrifuged at 12000 rpm to take the supernatant and precipitate and prepared Western Blot samples. Western Blot sample processing method: Took 40 μL of supernatant, added 10 μL of loading buffer, used WB (loading volume of 5 μL) to determine the concentration of Amuc_1100, and the result was shown in FIG. 13D.

We unexpectedly discovered that in the case of aerobic fermentation (simulating the industrial conditions for strain production), the use of anaerobic promoter Pfnrs had more advantages in maintaining the activity of the strain to secrete Amuc_1100 protein (by comparing CBT1108 and CBT1117). At the same time, adding one copy of the anaerobic promoter strain to the constitutive promoter strain (CBT1109) can increase the secretion of Amuc_1100 protein. However, if the number of copies of the anaerobic and aerobic promoters was further increased to four (see strain CBT1110), it did not bring more benefits, rather the expression level decreased sharply.

Next, the effects of Pfnrs promoters with different copy numbers (such as two copies, three copies and four copies) were studied. The strains constructed and/or tested were as follows: CBT1108 (two copies), CBT1111 (three copies) and CBT1112 (four copies). Experimental method: CBT1108, CBT1111 and CBT1112 single colonies were added to 50 mL of LB medium supplemented with 1% Glu, and after aerobic culture at 37° C. at 220 rpm for 2 hours, they were transferred to an anaerobic incubator to continue the culture. The total culture time was 8h, during which the bacterial solution was taken at 6h and 8h respectively, and the supernatant and precipitate were collected by centrifuging at 12000 rpm and Western Blot samples were prepared. The Western Blot sample processing method was: took 20 μL of supernatant, added 10 μL of loading buffer and 20 μL of PBS buffer, and used WB (loading volume of 5 μL) to determine the concentration of Amuc_1100. The results were shown in FIG. 13E. Under aerobic to anaerobic conditions, the culture supernatant of the strain having three copies and four copies of the Pfnrs promoter contained more Amuc_1100 protein than the strain having two copies of the Pfnrs promoter. However, further analysis of the growth status of the strains showed that the two strains containing three-copy and four-copy anaerobic promoters had maintained a very low OD₆₀₀ value of the bacterial solution shortly after the anaerobic culture (see FIG. 13F). Further, it was found that bacterial lysis occurred in the culture medium. This was probably due to the excessive expression of Amuc_1100 caused by too many copies of anaerobic promoter, which brought greater pressure and made the bacteria unable to maintain the normal form and ultimately led to lysis.

6.2.3 The Effect of Knocking Out Outer Membrane Protein

1) The LPP Protein Knock Out

Knocked out the LPP protein in the engineered bacteria according to the protocol in Example 5.8. The overnight cultured CBT1103 and CBT1113 were inoculated into 50 mL LB medium at a ratio of 1%, cultured at 37° C. at 220 rpm for 7 hours and centrifuged at 12000 rpm to collect the supernatant and precipitate and prepare Western Blot samples. Western Blot sample processing method: took 40 μL of supernatant, added 10 μL of loading buffer, and used WB (loading volume of 10 μL) to determine the concentration of Amuc_1100. The results were shown in FIG. 13G. CBT1103, which has two copies of Amuc_1100 expression cassette and has LPP knocked out, secreted even more Amuc_1100 than CBT1113 which has three copies of Amuc_1100 expression cassette and has intact LPP, suggesting that knocking out LPP could effectively promote the secretion of the Amuc_1100 protein.

2) the Effect of ompT Gene Knockout on the Secretion of Amuc_1100 Protein

Experimental method: the overnight cultured CBT1114 and CBT1115 bacterial solutions were inoculated into 50 mL LB medium at a ratio of 1%, cultured at 37° C. 220 rpm for 7 hours, and then centrifuged at 12000 rpm to take the supernatant and precipitate to prepare Western Blot samples. Western Blot sample processing method: took 40 μL of supernatant, added 10 μL of loading buffer, and used WB (loading volume of 10 μL) to determine the concentration of Amuc_1100.

As shown in FIG. 13H, CBT1114 without ompT gene knockout had obvious double bands, while the double bands of CBT1115 with ompT gene knockout were significantly weakened. It suggested that knocking out ompT gene could effectively prevent the degradation of the Amuc 1100 protein.

6.2.4 Chaperone

The effect of molecular chaperones on the expression of Amuc_1100 protein. Experimental method: the overnight cultured CBT1103 and CBT1116 were inoculated into 50 mL LB medium at a ratio of 1%, culture at 37° C. at 220 rpm for 7 hours and centrifuged at 12000 rpm to collect the supernatant and precipitate and prepared Western Blot samples. Western Blot sample processing method: took 40 μL of supernatant, added 15 μL of loading buffer, and used WB (loading volume of 10 μL) to determine the concentration of Amuc_1100.

As shown in FIG. 131, whether or not to use molecular chaperones had no significant effect on the secretion of Amuc_1100 protein.

Cistrons

(1) Construction of EcN_agaI/rsmI_J23101_MBP_usp45_Amuc_1100

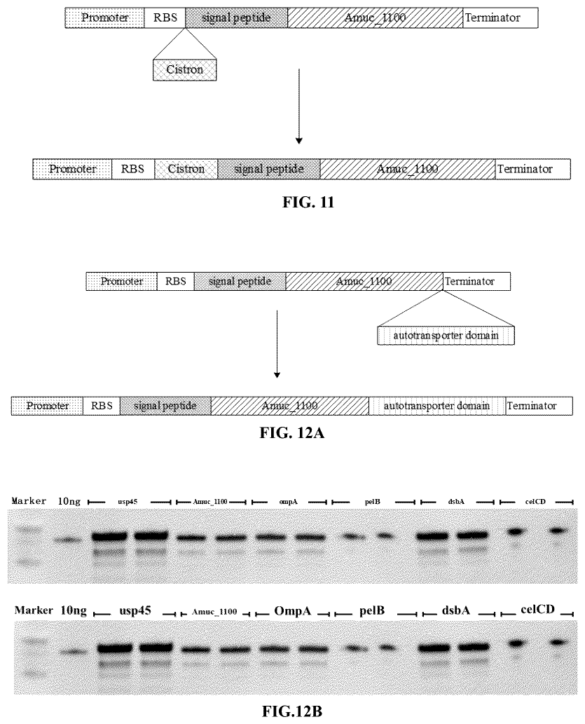

Cistrons were used to increase the transcription of Amuc_1100 (31-317), any cistron listed in Table 2 can be inserted to the Amuc_1100 (31-317) expression cassette as shown in FIG. 11. Primers used for the construction of EcN_agaI/rsmI_J23101_MBP_usp45_Amuc_1100 are listed in Table 7 below.

TABLE 7
agaI/rsmI_J23101_MBP_usp45_Amuc_1100 primers

| SEQ ID NO. | Name | Sequence |
|---|---|---|
| 119 | agaI/rsmI_LH-F | gccgcgcacgctgattttttatgaatc |
| 120 | agaI/rsmI_LH-R | ctcgagacgtcgacaagctctagagtagcgctattcctgctgcga |
| 121 | J23101_MBP-F | cgctactctagagcttgtcgacgtctc |
| 122 | J23101_MBP-R | cctccttctgtaccagtttacctgcttcgattttcataagcttctttctcctctttcct |
| 123 | MBP_usp45_F | agcaggtaaactggtacagaaggaggttaactgatgaagaaaaagatcattagct |
| 124 | MBP_usp45-R | ctacgcttgctgttaacaatcgcataaacaccgctcagcg |
| 125 | Amuc_1100-F | ctgagcggtgtttatgcgattgttaacagcaagcgtagcg |
| 126 | Amuc_1100-R | cgatctacactagcactatcgagctcgttattagtggtgg |
| 127 | Terminator-F | ccaccactaataacgagctcgatagtgctagtgtagatcg |
| 128 | Terminator-R | ctgcagcggccgcttctagtat |
| 129 | agaI/rsmI_RH-F | tactagaagcggccgctgcagtcacgcatgtgaaatggttatgc |
| 130 | agaI/rsmI_RH-R | gtgaagaccagctcatcagctttcgc |

Sat Secretion System

Construction of EcN agaI/rsmI_J23101_sat Amuc_1100_β domain

Sat secretion system, which uses the autotransporter domain (P3 domain) as shown in FIG. 12A was used to increase the secretion of Amuc_1100 (31-317). The primers used for the construction of EcN_agaI/rsmI_J23101_sat_Amuc_1100_β domain are listed in Table 8 below.

TABLE 8
agaI/rsmI_J23101_sat_Amuc_1100_β domain Primers

| SEQ ID NO. | Name | Sequence |
|---|---|---|
| 131 | agaI/rsmI_LH-F | gccgcgcacgctgattttttatgaatc |
| 132 | agaI/rsmI_LH-R | ctcgagacgtcgacaagctctagagtagcgctattcctgctgcga |
| 133 | J23101_sat-F | cgctactctagagcttgtcgacgtctc |
| 134 | J23101_sat-R | attcataagcttctttctcctctttcctc |
| 135 | sat_F | ctcgaggaaagaggagaaagaagcttatgaataaaatatact |
| | | cccttaaatatagtgc |
| 136 | sat-R | tgcatttactgtaccggcaaca |
| 137 | Amuc_1100-F | tctctggctgttgccggtacagtaaatgcaattgttaacagcaa |
| | | gcgtagcg |
| 138 | Amuc_1100-R | cgcagatcacccatacgtttgttaaggttgttgtggtggtggtg |
| | | atgatgatct |
| 139 | β domain-F | aaccttaacaaacgtatgggtgat |
| 140 | β domain-R | cactatcgagctcgttatcagaaagagtaacggaagttggcg |
| 141 | Terminator-F | acttccgttactctttctgataacgagctcgatagtgctagtg |
| 142 | Terminator-R | ctgcagcggccgcttctagtat |
| 143 | agaI/rsmI_RH-F | tactagaagcggccgctgcagtcacgcatgtgaaatggttatgc |
| 144 | agaI/rsmI_RH-R | gtgaagaccagctcatcagctttcgc |

### Example 7. Secreted Amuc_1100 (31-317) from Genetically Engineered EcN Stimulated TLR2 Signal

The genetically engineered bacteria EcN strain CBT1108 obtained in Example 6 were cultured, and the culture media was collected at late exponential growth stage. The Amuc_100 (31-317) protein was isolated from the culture media by his-tag antibody-containing resin column. The endotoxin was removed from the isolated Amuc_1100 (31-317) protein.

The Amuc_1100 (31-317) activity was tested by TLR2 reporter assay. Specifically, HEK293/TLR2 reporter cells were stimulated in 96-well plate with certain amount of protein for 24 hours, the reporter enzyme's substrate was added into the cell media and the activity was measured by colorimetric method.

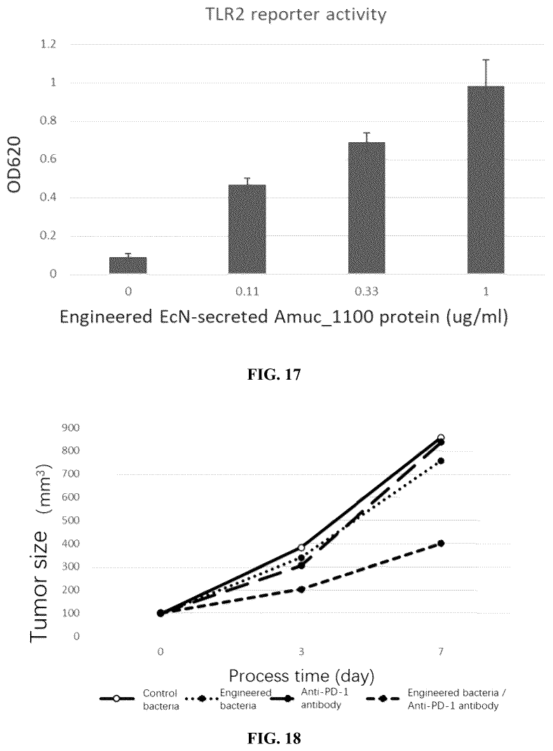

As shown in FIG. 17, the secreted Amuc_1100 (31-317) from genetically engineered EcN stimulated TLR2 signal. The engineered EcN provided in the present disclosure can significantly increase the activity of Amuc_1100, for example, stimulating the activity of TLR2 signals.

### Example 8. Impact of the Combination of Genetically Engineered EcN Encoding Amuc 1100 (31-317) (or its Mutants) and Anti-PD-1 Antibody on Inhibiting Tumor Growth in a Breast Cancer Mouse Model

The genetically engineered EcN strain tested in this Example is CBT1108. The MMTV-PyVT transgenic C57BL/6 female mice and EMT-6 orthotopic mice are used as breast cancer animal models. Mice at age of 9.5 weeks are randomly divided into 6 groups with 4-5 mice in each group and are subjected to agent treatment. The mice are treated with each agent when tumor size reaches to 100 mm³. Specifically, the mice in the control group are treated with PBS by oral gavage daily for 4 weeks, plus PBS i.v. injection daily after one week of oral gavage for 3 weeks. The mice in the five test groups are treated with (1) genetically engineered EcN encoding Amuc_1100 (31-317) by oral gavage daily for 4 weeks, (2) genetically engineered EcN encoding Amuc_1100 (31-317, Y289A) protein by oral gavage daily for 4 weeks, (3) anti-PD-1 antibody (InVivoMAb anti-mouse PD-1 (CD279) (Clone RMP1-14) (BioXcell, catalog #BE0146)) by i.v. injection every three days for 3 weeks, (4) genetically engineered EcN encoding Amuc_1100 (31-317) by oral gavage daily for one week, and then anti-PD-1 antibody (i.v. injection every three days) simultaneously with genetically engineered EcN encoding Amuc_1100 (31-317) (oral gavage daily) for another 3 weeks, (5) genetically engineered EcN encoding Amuc 1100 (31-317, Y289A) by oral gavage daily for one week, and then anti-PD-1 antibody (i.v. injection every three days) simultaneously with genetically engineered EcN encoding Amuc_1100 (31-317, Y289A) (oral gavage daily) for another 3 weeks. Tumor growth rate of each mouse is measured twice every week. At endpoint of the study, tumor tissues are collected and fixed in formalin. H&E staining is performed to determine the tumor cells in the tissue.

### Example 9. Impact of the Combination of Genetically Engineered EcN Encoding Amuc 1100 (31-317) and Anti-PD-1 Antibody on Inhibiting Tumor Growth in a Breast Cancer Mouse Model

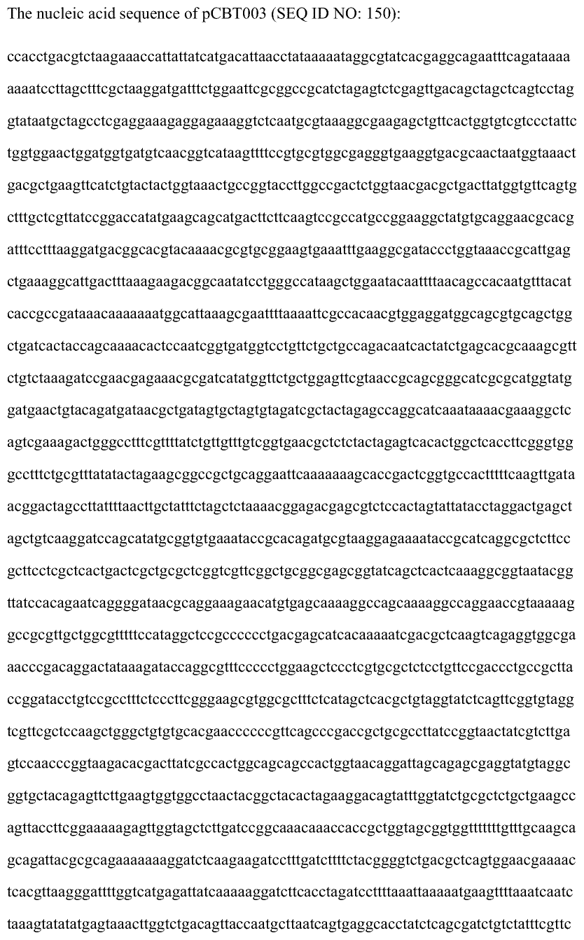

The genetically engineered EcN strain tested in this Example is CBT1117. The MMTV-PyMT tumor mass was transplanted into the mammary fat pad of C57BL/6 female mice to establish the MMTV-PyMT orthotopic breast tumor model. On the 13th day after inoculation, the average tumor volume was about 100 mm³, which was divided into 4 groups by random block method: the EcN (control bacteria) group without genetic modification, the EcN (engineered bacteria) group expressing two Amuc_1100 expression cassettes, anti-mPD-1 antibody (PD-1 antibody) group, combination therapy group (engineered bacteria/anti-PD-1 antibody), 5 mice in each group. The bacteria is given by intragastric administration with a volume of 200 μL/8×10\hat{ }10 CFU/mouse once a day. The anti-PD-1 antibody was given intraperitoneally at a dose of 10 mg/kg every 3 days for a total of 7 days. The tumor size of each mouse was measured every three days. As shown in FIG. 18, the combinatory use of engineered bacteria encoding Amuc_1100 and anti-PD-1 antibody significantly inhibited tumor growth.

### Example 10. Impact of the Combination of Genetically Engineered EcN Encoding Amuc 1100 (31-317) (or its Mutants) and Anti-PD-1 Antibody on Body Weight Change and Immune Cell Infiltration in Tumor

The mice are grouped and treated by similar methods in Example 9. Body weight of each mouse is measured twice every week. At the endpoint of the study, tumor tissues are harvested for following up analysis of the distribution of immune cells. At the endpoint of the study, tumor tissues are harvested for following up analysis of the distribution of immune cells (including macrophages, Tregs, CD4+ T cells, CD8+ T cells, etc.).

### Example 11. Impact of the Combination of Genetically Engineered EcN Encoding Amuc 1100 (31-317) and Other Immune Checkpoint Inhibitors (ICI) on Inhibiting Tumor Growth in a Breast Cancer Mouse Model

The MMTV-PyMT tumor mass was transplanted into the mammary fat pad of C57BL/6 female mice to establish the MMTV-PyMT orthotopic breast tumor model. On the 13th day after inoculation, the average tumor volume was about 100 mm³, which was divided into 4 groups by random block method: 1) the EcN (control bacteria) group without genetic modification, 2) the EcN (engineered bacteria) group expressing two Amuc_1100 expression cassettes, 3) the ICI group, which include one of the following: anti-PD-LI antibody group, anti-CTLA4 antibody group, anti-Lag-3 antibody group, anti-TIM-3 antibody group, anti-TIGIT antibody group, anti-CD47 antibody group, anti-CD137 antibody, anti-CD276 antibody group, or anti-GITR antibody group, and 4) combination therapy group (engineered bacteria/ICI). Each group has 5 mice. The bacteria are given by intragastric administration with a volume of 200 μL/8×10\hat{ }10 CFU/mouse once a day. The anti-ICI antibody was given intraperitoneally at a determined dose (eg. 10 mg/kg every 3 days for a total of 7 days). The tumor size of each mouse was measured every three days. Combinatory use of engineered bacteria encoding Amuc_1100 and ICIs significantly inhibit tumor growth.

1-111. (canceled)

112. A method of improving therapeutic response to cancer in a subject receiving treatment of an immune checkpoint modulator, comprising administering to a subject in need thereof an effective amount of Amuc_1100 or a genetically engineered host cell expressing the Amuc_1100, optionally in combination with an effective amount of an immune checkpoint modulator.

113. The method of claim 112, wherein the Amuc_1100 is a naturally-occurring Amuc_1100, or a functional equivalent thereof, wherein the functional equivalent retains at least partial activity in modulating gut immunity and/or in activating toll-like receptor 2 (TLR2); for example, the functional equivalent comprises a mutant, a fragment, a fusion, a derivative, an equivalent that improves the stability thereof, or any combination thereof of the naturally-occurring Amuc_1100; preferably, the Amuc_1100 comprises an amino acid sequence selected from the group consisting of SEQ ID NO: 1, SEQ ID NO: 2 and SEQ ID NO: 3, or an amino acid sequence having at least 80% sequence identity thereof yet retaining substantial activity in modulating gut immunity and/or in activating toll-like receptor 2 (TLR2); more preferably, the Amuc_1100 comprises Y289A mutation, wherein the numbering is relative to SEQ ID NO: 1.

114. The method of claim 112, wherein the immune checkpoint modulator is an antibody or an antigen-binding fragment thereof or a chemical compound against an immune checkpoint molecule; for example, the immune checkpoint modulator comprises an inhibitor of one or more immunoinhibitory checkpoints selected from the group consisting of LAG3 (CD223), A2AR, B7-H3 (CD276), B7-H4 (VTCN1), BTLA (CD272), BTLA, CD160, CTLA-4 (CD152), IDO1, IDO2, TDO, KIR, LAIR-1, NOX2, PD-1, PD-L1, PD-L2, TIM-3, VISTA, SIGLEC-7 (CD328), TIGIT, PVR (CD155), TGFβ, SIGLEC9 (CD329), and any combination thereof; preferably, the immune checkpoint modulator is an antibody or an antigen-binding fragment thereof or a chemical compound that inhibits or reduces interaction between PD-1 and any of its ligands.

115. The method of claim 112, wherein the subject has shown poor response, or resistance to the immune checkpoint modulator; for example, the subject is determined to have de novo or acquired resistance to the immune checkpoint modulator.

116. The method of claim 112, wherein the subject is a cancer patient, wherein the cancer is selected from the group consisting of colorectal cancer, breast cancer, ovarian cancer, pancreatic cancer, gastric cancer, prostate cancer, renal cancer, cervical cancer, myeloma cancer, lymphoma cancer, leukemia cancer, thyroid cancer, endometrial cancer, uterine cancer, bladder cancer, neuroendocrine cancer, head and neck cancer, liver cancer, nasopharyngeal cancer, testicular cancer, small cell lung cancer, non-small cell lung cancer, melanoma, basal cell cancer, skin cancer, squamous cell skin cancer, dermatofibrosarcoma protuberans, Merkel cell carcinoma, glioblastoma, glioma, sarcoma and mesothelioma.

117. The method of claim 112, wherein the administration of the Amuc_1100 or genetically engineered host cell expressing the Amuc_1100 is via oral administration; preferably, the administration of the Amuc_1100 or genetically engineered host cell expressing the Amuc_1100 is before, after, or simultaneously with the immune checkpoint modulator.

118. The method of claim 112, wherein the genetically engineered host cell comprises an exogenous expression cassette comprising a nucleotide sequence that encodes Amuc_1100 operably linked to a signal peptide, optionally, the genetically engineered host cell secrets at least 10 ng Amuc_1100 from 1×10⁹ CFU of the genetically engineered host cell, wherein the exogenous expression cassette is integrated in a plasmid of the genetically engineered host cell, or integrated in the genome of the genetically engineered host cell;
the exogenous expression cassette comprises a nucleotide sequence that encodes Amuc_1100 operably linked to a signal peptide, wherein the signal peptide is operably linked at the N-terminus of the Amuc_1100; preferably, the signal peptide comprises an amino acid sequence selected from the group consisting of SEQ ID NOs: 76-82 and homologous sequences thereof having at least 80% sequence identity; more preferably, the signal peptide is Usp45 signal peptide.

119. The method of claim 118, wherein the signal peptide can be processed by a secretion system present in the host cell, preferably, the secretion system is engineered and/or optimized such that at least one outer membrane protein encoding gene is deleted, inactivated, or suppressed; more preferably, the outer membrane protein is selected from the group consisting of: OmpC, OmpA, OmpF, OmpT, pldA, pagP, tolA, Pal, To1B, degS, mrcA and Ipp; and/or
the secretion system is engineered such that at least one Chaperone protein encoding gene is amplified, overexpressed or activated; preferably, the Chaperone protein is selected from the group consisting of: dsbA, dsbC, dnaK, dnaJ, grpE, groES, groEL, tig, fkpA, surA, skp, PpiD and DegP.

120. The method of claim 118, wherein the expression cassette further comprises one or more regulatory elements selected from the group consisting of: a promoter, a ribosome binding site (RBS), a cistron, a terminator, and any combination thereof; preferably, the promoter is a constitutive promoter, or an inducible promoter; more preferably, the constitutive promoter comprises SEQ ID NO: 15 or the inducible promoter comprises SEQ ID NO: 58; and/or
the RBS comprises a nucleotide sequence selected from the group consisting of SEQ ID NOs: 70-73 and homologous sequences thereof having at least 80% sequence identity; and/or
the cistron comprises a nucleotide sequence selected from the group consisting of SEQ ID NOs: 66-69 and homologous sequences thereof having at least 80% sequence identity; and/or
the terminator is T7 terminator, more preferably, the promoter is rrnB_T1_T7Te terminator, even more preferably, the promoter comprises a nucleotide sequence as shown in SEQ ID NO: 74.

121. The method of claim 112, wherein the host cell comprises a probiotic microorganism or a non-pathogenic microorganism, preferably, the probiotic microorganism is a probiotic bacterium or a probiotic yeast; more preferably,
the probiotic bacterium is selected from the group consisting of Bacteroides, Bifidobacterium, Clostridium, Escherichia, Lactobacillus and Lactococcus, optionally, the probiotic bacterium is of the genus Escherichia, and optionally, the probiotic bacterium is of the species Escherichia coli strain Nissle 1917 (EcN); and/or
the probiotic yeast is selected from the group consisting of Saccharomyces cerevisiae, Candida utilis, Kluyveromyces lactis, and Saccharomyces carlsbergensis.

122. An engineered host cell comprising an exogenous expression cassette, wherein the exogenous expression cassette comprising a nucleotide sequence that encodes Amuc_1100 operably linked to a signal peptide, optionally, the genetically engineered host cell secrets at least 10ng Amuc_1100 from 1×10⁹ CFU of the genetically engineered host cell; optionally, the signal peptide is Usp45 signal peptide, more preferably, the host cell is Escherichia coli strain Nissle 1917 (EcN).

123. The genetically engineered host cell of claim 122, wherein the Amuc_1100 is a naturally-occurring Amuc_1100, or a functional equivalent thereof, wherein the functional equivalent retains at least partial activity in modulating gut immunity and/or in activating toll-like receptor 2 (TLR2).

124. The genetically engineered host cell of claim 122, wherein the Amuc_1100 comprises Y289A mutation.

125. The genetically engineered host cell of claim 122, wherein the Amuc_1100 comprises an amino acid sequence selected from the group consisting of SEQ ID NOs: 1-3 or a homologous sequence thereof having at least 80% sequence identity.

126. A method of constructing an expression vector comprising a polynucleotide encoding a target polypeptide, wherein the expression vector is suitable for expression in E. Coli to allow expression and secretion of target polypeptide from the E. Coli, comprising the step of using Usp45 signal peptide for the expression vector; preferably, the Usp45 signal peptide comprises the sequence as set forth in SEQ ID NO: 59.

127. The method of claim 126, wherein the E. Coli is a commensal strain of E. Coli; preferably, the commensal strain is E. coli Nissle 1917 strain.

128. The method of claim 126, wherein the target polypeptide comprises Amuc_1100, preferably, the Amuc_1100 is a naturally-occurring Amuc_1100, or a functional equivalent thereof; or the target polypeptide is a polypeptide other than Amuc_1100.


SEQUENCE LISTING (214 sequences)

SEQ ID NO: 1 - protein, 317 aa, Artificial Sequence
  synthetic
MSNWITDNKPAAMVAGVGLLLFLGLSATGYIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSED

SEQ ID NO: 2 - protein, 288 aa, Artificial Sequence
  synthetic
MIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSED

SEQ ID NO: 3 - protein, 238 aa, Artificial Sequence
  synthetic
MSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSED

SEQ ID NO: 4 - protein, 288 aa, Artificial Sequence
  synthetic
MIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPAMGKEQVFVQVSLNLVHFNQPKAQEPSED

SEQ ID NO: 5 - protein, 238 aa, Artificial Sequence
  synthetic
MSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPAMGKEQVFVQVSLNLVHFNQPKAQEPSED

SEQ ID NO: 6 - protein, 294 aa, Artificial Sequence
  synthetic
MIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSEDHHHHHH

SEQ ID NO: 7 - protein, 244 aa, Artificial Sequence
  synthetic
MSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSEDHHHHHH

SEQ ID NO: 8 - protein, 294 aa, Artificial Sequence
  synthetic
MIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPAMGKEQVFVQVSLNLVHFNQPKAQEPSEDHHHHHH

SEQ ID NO: 9 - protein, 244 aa, Artificial Sequence
  synthetic
MSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPAMGKEQVFVQVSLNLVHFNQPKAQEPSEDHHHHHH

SEQ ID NO: 10 - protein, 299 aa, Artificial Sequence
  synthetic
MHHHHHHDDDDKIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSED

SEQ ID NO: 11 - protein, 249 aa, Artificial Sequence
  synthetic
MHHHHHHDDDDKSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSED

SEQ ID NO: 12 - protein, 299 aa, Artificial Sequence
  synthetic
MHHHHHHDDDDKIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPAMGKEQVFVQVSLNLVHFNQPKAQEPSED

SEQ ID NO: 13 - protein, 249 aa, Artificial Sequence
  synthetic
MHHHHHHDDDDKSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPAMGKEQVFVQVSLNLVHFNQPKAQEPSED

SEQ ID NO: 14 - DNA, 35 bp, Artificial Sequence
  synthetic
ttgacagctagctcagtcctaggtataatgctagc

SEQ ID NO: 15 - DNA, 35 bp, Artificial Sequence
  synthetic
tttacagctagctcagtcctaggtattatgctagc

SEQ ID NO: 16 - DNA, 35 bp, Artificial Sequence
  synthetic
ttgacagctagctcagtcctaggtactgtgctagc

SEQ ID NO: 17 - DNA, 35 bp, Artificial Sequence
  synthetic
ctgatagctagctcagtcctagggattatgctagc

SEQ ID NO: 18 - DNA, 35 bp, Artificial Sequence
  synthetic
tttacagctagctcagtcctagggactgtgctagc

SEQ ID NO: 19 - DNA, 35 bp, Artificial Sequence
  synthetic
tttacggctagctcagtcctaggtacaatgctagc

SEQ ID NO: 20 - DNA, 35 bp, Artificial Sequence
  synthetic
tttatggctagctcagtcctaggtacaatgctagc

SEQ ID NO: 21 - DNA, 35 bp, Artificial Sequence
  synthetic
ttgacagctagctcagtcctagggattgtgctagc

SEQ ID NO: 22 - DNA, 77 bp, Artificial Sequence
  synthetic
aaagtgttttgtaatcataaagaaatattaaggtggggtaggaatagtataatatgtttattcaaccgaacttaatg

SEQ ID NO: 23 - DNA, 176 bp, Artificial Sequence
  synthetic
gggccgagcatgtggaaacacctctgccaggtcaagcccgcagccatgccgcatgtttttttcttgaccgcccgggcagaaccgtcgggcggagccgtgtcatgctccacacacgctacatggaaagaaacaagagatttttgctagccacagttcggtttttggtgtaactaatc

SEQ ID NO: 24 - DNA, 339 bp, Artificial Sequence
  synthetic
gtaaatttaggattaatcctggaactttttttgtcgcccagccaatgctttcagtcgtgactaattttccttgcggaggcttgtctgaagcggtttccgcgattttcttctgtaaattgtcgctgacaaaaaagattaaacgtaccttatacaagacttttttttcatatgcctgacggagttcacacttgtaagttttcaactacgttgtagactttacatcgccaggggtgctcggcataagccgaagatatcggtagagttaatattgagcagatccccggtgaaggatttaaccgtgttatctcgttggagatattcatggcgtattttggatga

SEQ ID NO: 25 - DNA, 35 bp, Artificial Sequence
  synthetic
ttgacggctagctcagtcctaggtacagtgctagc

SEQ ID NO: 26 - DNA, 35 bp, Artificial Sequence
  synthetic
ttgacagctagctcagtcctaggtattgtgctagc

SEQ ID NO: 27 - DNA, 35 bp, Artificial Sequence
  synthetic
tttacggctagctcagtcctaggtactatgctagc

SEQ ID NO: 28 - DNA, 30 bp, Artificial Sequence
  synthetic
gtttatacataggcgagtactctgttatgg

SEQ ID NO: 29 - DNA, 30 bp, Artificial Sequence
  synthetic
ggtttcaaaattgtgatctatatttaacaa

SEQ ID NO: 30 - DNA, 35 bp, Artificial Sequence
  synthetic
ttgacggctagctcagtcctaggtattgtgctagc

SEQ ID NO: 31 - DNA, 35 bp, Artificial Sequence
  synthetic
ttgacagctagctcagtcctagggactatgctagc

SEQ ID NO: 32 - DNA, 35 bp, Artificial Sequence
  synthetic
tttatagctagctcagcccttggtacaatgctagc

SEQ ID NO: 33 - DNA, 35 bp, Artificial Sequence
  synthetic
ctgatggctagctcagtcctagggattatgctagc

SEQ ID NO: 34 - DNA, 35 bp, Artificial Sequence; identical to SEQ ID NO: 17
  synthetic

SEQ ID NO: 35 - DNA, 35 bp, Artificial Sequence
  synthetic
ttgacggctagctcagtcctaggtatagtgctagc

SEQ ID NO: 36 - DNA, 35 bp, Artificial Sequence
  synthetic
ctgacagctagctcagtcctaggtataatgctagc

SEQ ID NO: 37 - DNA, 35 bp, Artificial Sequence
  synthetic
tttacggctagctcagccctaggtattatgctagc

SEQ ID NO: 38 - DNA, 35 bp, Artificial Sequence
  synthetic
tttacggctagctcagtcctaggtatagtgctagc

SEQ ID NO: 39 - DNA, 30 bp, Artificial Sequence
  synthetic
agaggttccaactttcaccataatgaaaca

SEQ ID NO: 40 - DNA, 30 bp, Artificial Sequence
  synthetic
caccttcgggtgggcctttctgcgtttata

SEQ ID NO: 41 - DNA, 30 bp, Artificial Sequence
  synthetic
ggctagctcagtcctaggtactatgctagc

SEQ ID NO: 42 - DNA, 30 bp, Artificial Sequence
  synthetic
aaagtgtgacgccgtgcaaataatcaatgt

SEQ ID NO: 43 - DNA, 30 bp, Artificial Sequence
  synthetic
ggctagctcagtcctaggtattatgctagc

SEQ ID NO: 44 - DNA, 30 bp, Artificial Sequence
  synthetic
taaacaactaacggacaattctacctaaca

SEQ ID NO: 45 - DNA, 178 bp, Artificial Sequence
  synthetic
aagctgttgtgaccgcttgctctagccagctatcgagttgtgaaccgatccatctagcaattggtctcgatctagcgataggcttcgatctagctatgtatcactcattaggcaccccaggctttacactttatgcttccggctcgtataatgtgtggtgctggttagcgcttgctat

SEQ ID NO: 46 - DNA, 190 bp, Artificial Sequence
  synthetic
aagctgttgtgaccgcttgctctagccagctatcgagttgtgaaccgatccatctagcaattggtctcgatctagcgataggcttcgatctagctatgtagaaacgccgtgtgctcgatcgcttgataaggtccacgtagctgctataattgcttcaacagaacatattgactatccggtattacccggc

SEQ ID NO: 47 - DNA, 30 bp, Artificial Sequence
  synthetic
catacgccgttatacgttgtttacgctttg

SEQ ID NO: 48 - DNA, 73 bp, Artificial Sequence
  synthetic
taattcctaatttttgttgacactctatcgttgatagagttattttaccactccctatcagtgatagagaaaa

SEQ ID NO: 49 - DNA, 44 bp, Artificial Sequence
  synthetic
ttgacaattaatcatccggctcgtataatgtgtggaattgtgag

SEQ ID NO: 50 - DNA, 52 bp, Artificial Sequence
  synthetic
cccaggctttacactttatgcttccggctcgtataatgtgtggaattgtgag

SEQ ID NO: 51 - DNA, 30 bp, Artificial Sequence
  synthetic
tgctagctactagagattaaagaggagaaa

SEQ ID NO: 52 - DNA, 30 bp, Artificial Sequence
  synthetic
ggctagctcagtcctaggtacagtgctagc

SEQ ID NO: 53 - DNA, 30 bp, Artificial Sequence
  synthetic
ccccgaaagcttaagaatataattgtaagc

SEQ ID NO: 54 - DNA, 30 bp, Artificial Sequence
  synthetic
atatatatatatatataatggaagcgtttt

SEQ ID NO: 55 - DNA, 74 bp, Artificial Sequence
  synthetic
tccctatcagtgatagagattgacatccctatcagtgatagagatactgagcacatcagcaggacgcactgacc

SEQ ID NO: 56 - DNA, 68 bp, Artificial Sequence
  synthetic
attccactaatttattccatgtcacacttttcgcatctttgttatgctatggttatttcataccataa

SEQ ID NO: 57 - DNA, 76 bp, Artificial Sequence
  synthetic
ataaatgtgagcggataacaattgacattgtgagcggataacaagatactgagcacatcagcaggacgcactgacc

SEQ ID NO: 58 - DNA, 65 bp, Artificial Sequence
  synthetic
aaaaacgccgcaaagtttgagcgaagtcaataaactctctacccattcagggcaatatctctctt

SEQ ID NO: 59 - DNA, 81 bp, Artificial Sequence
  synthetic
atgaagaaaaagatcattagcgcgatcctgatgagcaccgtgattctgagcgcggcggcgccgctgagcggtgtttatgcg

SEQ ID NO: 60 - DNA, 63 bp, Artificial Sequence
  synthetic
atgaagaaaaccgcgattgcgattgcggtggcgctggcgggtttcgcgaccgttgcgcaggcg

SEQ ID NO: 61 - DNA, 57 bp, Artificial Sequence
  synthetic
atgaagaaaatctggctggcgctggcgggtctggtgctggcgttcagcgcgagcgcg

SEQ ID NO: 62 - DNA, 66 bp, Artificial Sequence
  synthetic
atgaagtacctgctgccgaccgcggcggcgggtctgctgctgctggcggcgcagccggcgatggcg

SEQ ID NO: 63 - DNA, 63 bp, Artificial Sequence
  synthetic
atggaaggaaacactcgtgaagacaattttaaacatttattaggtaatgacaatgttaaacgc

SEQ ID NO: 64 - DNA, 147 bp, Artificial Sequence
  synthetic
atgaataaaatatactcccttaaatatagtgctgccactggcggactcattgctgtttctgaattagcgaaaagagtttctggtaaaacaaaccgaaaacttgtagcaacaatgttgtctctggctgttgccggtacagtaaatgca

SEQ ID NO: 65 - DNA, 90 bp, Artificial Sequence
  synthetic
atgagcaactggatcaccgacaacaaaccggctgcgatggttgcgggtgtgggcctgctgctgttcctgggtctgagcgcgaccggctac

SEQ ID NO: 66 - DNA, 33 bp, Artificial Sequence
  synthetic
atgcgtaaaggcgaagagaaggaggttaactga

SEQ ID NO: 67 - DNA, 54 bp, Artificial Sequence
  synthetic
atgaaagcaattttcgtactgaaacatcttaatcatgctaaggaggttttctaa

SEQ ID NO: 68 - DNA, 33 bp, Artificial Sequence
  synthetic
atgattatgtccggttataaggaggttaactga

SEQ ID NO: 69 - DNA, 45 bp, Artificial Sequence
  synthetic
atgaaaatcgaagcaggtaaactggtacagaaggaggttaactga

SEQ ID NO: 70 - DNA, 22 bp, Artificial Sequence
  synthetic
ggaggaaaaattaaaaaagaac

SEQ ID NO: 71 - DNA, 12 bp, Artificial Sequence
  synthetic
aaagaggagaaa

SEQ ID NO: 72-73 - skipped (2 entries)

SEQ ID NO: 74 - DNA, 186 bp, Artificial Sequence
  synthetic
cgagctcgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcag

SEQ ID NO: 75 - DNA, 173 bp, Artificial Sequence
  synthetic
cgagctcgatagtgctagtgtagatcgctactagagccaggcatcaaataaaacgaaagactgggcctttcgttttatctgttgtttgtcggtgaacgctctctactagagtcacactggctcaccttcgggtgggcctttctgcgtttatatactagaagcggccgctgcag

SEQ ID NO: 76 - protein, 27 aa, Artificial Sequence
  synthetic
MKKKIISAILMSTVILSAAAPLSGVYA

SEQ ID NO: 77 - protein, 21 aa, Artificial Sequence
  synthetic
MKKTAIAIAVALAGFATVAQA

SEQ ID NO: 78 - protein, 19 aa, Artificial Sequence
  synthetic
MKKIWLALAGLVLAFSASA

SEQ ID NO: 79 - protein, 22 aa, Artificial Sequence
  synthetic
MKYLLPTAAAGLLLLAAQPAMA

SEQ ID NO: 80 - protein, 21 aa, Artificial Sequence
  synthetic
MEGNTREDNFKHLLGNDNVKR

SEQ ID NO: 81 - protein, 49 aa, Artificial Sequence
  synthetic
MNKIYSLKYSAATGGLIAVSELAKRVSGKTNRKLVATMLSLAVAGTVNA

SEQ ID NO: 82 - protein, 30 aa, Artificial Sequence
  synthetic
MSNWITDNKPAAMVAGVGLLLFLGLSATGY

SEQ ID NO: 83 - DNA, 20 bp, Artificial Sequence
  synthetic
ggcgagttaacgacgacaca

SEQ ID NO: 84 - DNA, 20 bp, Artificial Sequence
  synthetic
cgttgaactgggtgtggaat

SEQ ID NO: 85 - DNA, 20 bp, Artificial Sequence
  synthetic
ctgaaaccgctgcggcgatg

SEQ ID NO: 86 - DNA, 20 bp, Artificial Sequence
  synthetic
gttcgcagtggaagtaccgt

SEQ ID NO: 87 - DNA, 20 bp, Artificial Sequence
  synthetic
ctgtgcaaacaagtgtctca

SEQ ID NO: 88 - DNA, 20 bp, Artificial Sequence
  synthetic
gccggaagacactatgaagc

SEQ ID NO: 89 - DNA, 20 bp, Artificial Sequence
  synthetic
tcgcacagcgtgtaccacag

SEQ ID NO: 90 - DNA, 20 bp, Artificial Sequence
  synthetic
gaaggggaagaggcgcgcgt

SEQ ID NO: 91 - DNA, 20 bp, Artificial Sequence
  synthetic
cggtttagttcacagaagcc

SEQ ID NO: 92 - DNA, 20 bp, Artificial Sequence
  synthetic
ttgcgtattttcaaaaagcg

SEQ ID NO: 93 - DNA, 20 bp, Artificial Sequence
  synthetic
tcatcagagtaagtcggata

SEQ ID NO: 94 - DNA, 20 bp, Artificial Sequence
  synthetic
ctgaccaacgcttctttacc

SEQ ID NO: 95 - DNA, 20 bp, Artificial Sequence
  synthetic
ccgaagtccctgtgtgcttt

SEQ ID NO: 96 - DNA, 20 bp, Artificial Sequence
  synthetic
ccctgccactcacaccattc

SEQ ID NO: 97 - DNA, 20 bp, Artificial Sequence
  synthetic
ccttctcctggcaagcttac

SEQ ID NO: 98 - DNA, 20 bp, Artificial Sequence
  synthetic
cgacaagaagtacctgcaac

SEQ ID NO: 99 - DNA, 20 bp, Artificial Sequence
  synthetic
gttatcgtgatgcgcattct

SEQ ID NO: 100 - DNA, 20 bp, Artificial Sequence
  synthetic
taacacccagcccgatgccc

SEQ ID NO: 101 - DNA, 20 bp, Artificial Sequence
  synthetic
atattactggaacaacataa

SEQ ID NO: 102 - DNA, 20 bp, Artificial Sequence
  synthetic
ccgtgacgttatccgcacca

SEQ ID NO: 103 - DNA, 20 bp, Artificial Sequence
  synthetic
tatcgccttcatccacacga

SEQ ID NO: 104 - DNA, 20 bp, Artificial Sequence
  synthetic
ccgggctggatgtcttcgaa

SEQ ID NO: 105 - DNA, 20 bp, Artificial Sequence
  synthetic
gagggtgagccataatgaag

SEQ ID NO: 106 - DNA, 20 bp, Artificial Sequence
  synthetic
ggatatgtggggtaacgacg

SEQ ID NO: 107 - DNA, 20 bp, Artificial Sequence
  synthetic
acgttcaggctgctaaagat

SEQ ID NO: 108 - DNA, 1258 bp, Artificial Sequence
  synthetic
tctagagcttgtcgacgtctcgagtttacagctagctcagtcctaggtattatgctagcctcgaggaaagaggagaaagaagcttatgaagaaaaagatcattagcgcgatcctgatgagcaccgtgattctgagcgcggcggcgccgctgagcggtgtttatgcgggtagcagccatcatcaccaccaccacgacgacgacgacaaaattgttaacagcaagcgtagcgagctggataagaaaatcagcattgcggcgaaagagatcaagagcgcgaacgcggcggaaattaccccgagccgtagcagcaacgaggaactggagaaagaactgaaccgttacgcgaaggcggttggtagcctggaaaccgcgtataaaccgtttctggcgagcagcgcgctggtgccgaccaccccgaccgcgttccaaaacgagctgaaaacctttcgtgacagcctgatcagcagctgcaagaaaaagaacatcctgattaccgataccagcagctggctgggcttccaggtttacagcacccaagcgccgagcgtgcaggcggcgagcaccctgggttttgagctgaaagcgattaacagcctggttaacaagctggcggaatgcggcctgagcaaattcatcaaggtgtatcgtccgcagctgccgattgaaaccccggcgaacaacccggaggaaagcgacgaagcggatcaggcgccgtggaccccgatgccgctggagatcgcgttccagggtgaccgtgaaagcgttctgaaagcgatgaacgcgattaccggcatgcaagattacctgtttaccgtgaacagcatccgtattcgtaacgaacgtatgatgccgccgccaatcgcgaacccggctgcggcgaagccggctgcggcgcagccggcgaccggtgcggcgagcctgaccccggcggacgaggctgcggcgccggctgcgccggcgatccagcaagttattaaaccgtatatgggcaaggaacaggtgttcgttcaagtgagcctgaacctggtgcactttaaccagccgaaagcgcaagagccgagcgaagattgacgagctcgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcag

SEQ ID NO: 109-118 - skipped (10 entries)

SEQ ID NO: 119 - DNA, 27 bp, Artificial Sequence
  synthetic
gccgcgcacgctgattttttatgaatc

SEQ ID NO: 120 - DNA, 45 bp, Artificial Sequence
  synthetic
ctcgagacgtcgacaagctctagagtagcgctattcctgctgcga

SEQ ID NO: 121 - DNA, 27 bp, Artificial Sequence
  synthetic
cgctactctagagcttgtcgacgtctc

SEQ ID NO: 122 - DNA, 59 bp, Artificial Sequence
  synthetic
cctccttctgtaccagtttacctgcttcgattttcataagcttctttctcctctttcct

SEQ ID NO: 123 - DNA, 54 bp, Artificial Sequence
  synthetic
agcaggtaaactggtacagaaggaggttaactgatgaagaaaaagatcattagc

SEQ ID NO: 124 - DNA, 40 bp, Artificial Sequence
  synthetic
ctacgcttgctgttaacaatcgcataaacaccgctcagcg

SEQ ID NO: 125 - DNA, 40 bp, Artificial Sequence
  synthetic
ctgagcggtgtttatgcgattgttaacagcaagcgtagcg

SEQ ID NO: 126 - DNA, 40 bp, Artificial Sequence
  synthetic
cgatctacactagcactatcgagctcgttattagtggtgg

SEQ ID NO: 127 - DNA, 40 bp, Artificial Sequence
  synthetic
ccaccactaataacgagctcgatagtgctagtgtagatcg

SEQ ID NO: 128 - DNA, 22 bp, Artificial Sequence
  synthetic
ctgcagcggccgcttctagtat

SEQ ID NO: 129 - DNA, 44 bp, Artificial Sequence
  synthetic
tactagaagcggccgctgcagtcacgcatgtgaaatggttatgc

SEQ ID NO: 130 - DNA, 26 bp, Artificial Sequence
  synthetic
gtgaagaccagctcatcagctttcgc

SEQ ID NO: 131 - DNA, 27 bp, Artificial Sequence
  synthetic
gccgcgcacgctgattttttatgaatc

SEQ ID NO: 132 - DNA, 45 bp, Artificial Sequence; identical to SEQ ID NO: 120
  synthetic

SEQ ID NO: 133 - DNA, 27 bp, Artificial Sequence
  synthetic
cgctactctagagcttgtcgacgtctc

SEQ ID NO: 134 - DNA, 29 bp, Artificial Sequence
  synthetic
attcataagcttctttctcctctttcctc

SEQ ID NO: 135 - DNA, 58 bp, Artificial Sequence
  synthetic
ctcgaggaaagaggagaaagaagcttatgaataaaatatactcccttaaatatagtgc

SEQ ID NO: 136 - DNA, 22 bp, Artificial Sequence
  synthetic
tgcatttactgtaccggcaaca

SEQ ID NO: 137 - DNA, 52 bp, Artificial Sequence
  synthetic
tctctggctgttgccggtacagtaaatgcaattgttaacagcaagcgtagcg

SEQ ID NO: 138 - DNA, 54 bp, Artificial Sequence
  synthetic
cgcagatcacccatacgtttgttaaggttgttgtggtggtggtgatgatgatct

SEQ ID NO: 139 - DNA, 24 bp, Artificial Sequence
  synthetic
aaccttaacaaacgtatgggtgat

SEQ ID NO: 140 - DNA, 42 bp, Artificial Sequence
  synthetic
cactatcgagctcgttatcagaaagagtaacggaagttggcg

SEQ ID NO: 141 - DNA, 43 bp, Artificial Sequence
  synthetic
acttccgttactctttctgataacgagctcgatagtgctagtg

SEQ ID NO: 142 - DNA, 22 bp, Artificial Sequence
  synthetic
ctgcagcggccgcttctagtat

SEQ ID NO: 143 - DNA, 44 bp, Artificial Sequence; identical to SEQ ID NO: 129
  synthetic

SEQ ID NO: 144 - DNA, 26 bp, Artificial Sequence
  synthetic
gtgaagaccagctcatcagctttcgc

SEQ ID NO: 145 - protein, 296 aa, Artificial Sequence
  synthetic
MIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSEDLEHHHHHH

SEQ ID NO: 146 - protein, 296 aa, Artificial Sequence
  synthetic
MIVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPAMGKEQVFVQVSLNLVHFNQPKAQEPSEDLEHHHHHH

SEQ ID NO: 147 - DNA, 891 bp, Artificial Sequence
  synthetic
atgatcgtgaatagtaagcgtagcgaactggataagaagattagcattgccgccaaagaaattaaatctgcaaatgcagcagaaattacacctagtcgcagcagcaatgaagaactggagaaagaactgaatcgttatgcaaaggcagttggcagcctggaaaccgcctataaaccgtttctggcaagcagtgcactggttccgaccacacctaccgcatttcagaatgaactgaagactttccgtgatagtctgattagtagctgcaagaagaagaatattctgattaccgataccagtagttggctgggtttccaggtttatagcacccaggcaccgagcgtgcaggccgcaagtaccctgggtttcgaactgaaagccattaatagtctggtgaataaactggccgaatgtggcctgagcaaatttattaaagtttatcgtccgcagctgccgattgaaacacctgccaataatccggaagaaagcgatgaagcagatcaggcaccgtggacacctatgccgctggaaattgcatttcagggcgatcgtgaaagcgtgctgaaagccatgaatgccattaccggcatgcaggattatctgtttaccgtgaatagtattcgcattcgcaatgaacgcatgatgccgccgccgattgccaatccggcagccgcaaagccggcagccgcccagcctgcaaccggtgcagcaagcctgacacctgcagatgaagcagcagcaccggcagcacctgcaattcagcaggttattaaaccggccatgggcaaagaacaggtgtttgttcaggttagcctgaatctggtgcatttcaatcagccgaaagcacaggaaccgagcgaagatctcgagcaccaccaccaccaccactga

SEQ ID NO: 148 - DNA, 1245 bp, Artificial Sequence
  synthetic
tctagagcttgtcgacgtctcgagtttacagctagctcagtcctaggtattatgctagcctcgaggaaagaggagaaagaagcttatgaagaaaaagatcattagcgcgatcctgatgagcaccgtgattctgagcgcggcggcgccgctgagcggtgtttatgcgggtagcagccatcatcaccaccaccacgacgacgacgacaaaattgttaacagcaagcgtagcgagctggataagaaaatcagcattgcggcgaaagagatcaagagcgcgaacgcggcggaaattaccccgagccgtagcagcaacgaggaactggagaaagaactgaaccgttacgcgaaggcggttggtagcctggaaaccgcgtataaaccgtttctggcgagcagcgcgctggtgccgaccaccccgaccgcgttccaaaacgagctgaaaacctttcgtgacagcctgatcagcagctgcaagaaaaagaacatcctgattaccgataccagcagctggctgggcttccaggtttacagcacccaagcgccgagcgtgcaggcggcgagcaccctgggttttgagctgaaagcgattaacagcctggttaacaagctggcggaatgcggcctgagcaaattcatcaaggtgtatcgtccgcagctgccgattgaaaccccggcgaacaacccggaggaaagcgacgaagcggatcaggcgccgtggaccccgatgccgctggagatcgcgttccagggtgaccgtgaaagcgttctgaaagcgatgaacgcgattaccggcatgcaagattacctgtttaccgtgaacagcatccgtattcgtaacgaacgtatgatgccgccgccaatcgcgaacccggctgcggcgaagccggctgcggcgcagccggcgaccggtgcggcgagcctgaccccggcggacgaggctgcggcgccggctgcgccggcgatccagcaagttattaaaccgtatatgggcaaggaacaggtgttcgttcaagtgagcctgaacctggtgcactttaaccagccgaaagcgcaagagccgagcgaagattgacgagctcgatagtgctagtgtagatcgctactagagccaggcatcaaataaaacgaaagactgggcctttcgttttatctgttgtttgtcggtgaacgctctctactagagtcacactggctcaccttcgggtgggcctttctgcgtttatatactagaagcggccgctgcag

SEQ ID NO: 149 - DNA, 15460 bp, Artificial Sequence
  Synthetic
ctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcggatcttgctgtaggcataggcttggttatgccggtactgccgggcctcttgcgggattacgaaatcatcctgtggagcttagtaggtttagcaagatggcagcgcctaaatgtagaatgataaaaggattaagagattaatttccctaaaaatgataaaacaagcgttttgaaagcgcttgtttttttggtttgcagtcagagtagaatagaagtatcaaaaaaagcaccgactcggtgccactttttcaagttgataacggactagccttattttaacttgctatgctgttttgaatggttccaacaagattattttataacttttataacaaataatcaaggagaaattcaaagaaatttatcagccataaaacaatacttaatactatagaatgataacaaaataaactactttttaaaagaattttgtgttataatctatttattattaagtattgggtaatattttttgaagagatattttgaaaaagaaaaattaaagcatattaaactaatttcggaggtcattaaaactattattgaaatcatcaaactcattatggatttaatttaaactttttattttaggaggcaaaaatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaagtatattttagatgaagattatttcttaataactaaaaatatggtataatactcttaataaatgcagtaatacaggggcttttcaagactgaagtctagctgagacaaatagtgcgattacgaaattttttagacaaaaatagtctacgaggttttagagctatgctgttttgaatggtcccaaaactgcagcgcaatagttggcgaagtaatcgcaacatccgcattaaaatctagcgagggctttactaagctccgcaaaaaaccccgcccctgacagggcggggttttttcgcgcccgatccttggagcccttgccctcccgcacgatgatcgtgccgtgatcgaaatccagatccttgacccgcagttgcaaaccctcactgatccgcatgcttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatgtaggtcgttcgctccaagcgttttagagctagaaatagcaagttaaaataaggctagtccgttatcaacttgaaaaagtggcaccagaccccgttgatgataccgctgccttactgggtgcattagccagtctgaatgacctcgagttttagagctatgctgttttgaatggtcccaaaacttcagcacactgagacttgttgagttccatgttttagagctatgctgttttgaatggactccattcaacattgccgatgataacttgagaaagagggttaataccagcagtcggataccttcctattctttctgttaaagcgttttcatgttataataggcaaaagaagagtagtgtgatcgtccattccgacgtcgagccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgcctactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctcctgcagagatctgaattccctagagagacgaaagtgattgcgcctacccggatattatcgtgaggatgcgtcatcgccattgctccccaaatacaaaaccaatttcagccagtgcctcgtccattttttcgatgaactccggcacgatctcgtcaaaactcgccatgtacttttcatcccgctcaatcacgacataatgcaggccttcacgcttcatacgcgggtcatagttggcaaagtaccaggcattttttcgcgtcacccacatgctgtactgcacctgggccatgtaagctgactttatggcctcgaaaccaccgagccggaacttcatgaaatcccgggaggtaaacgggcatttcagttcaaggccgttgccgtcactgcataaaccatcgggagagcaggcggtacgcatactttcgtcgcgatagatgatcggggattcagtaacattcacgccggaagtgaattcaaacagggttctggcgtcgttctcgtactgttttccccaggccagtgctttagcgttaacttccggagccacaccggtgcaaacctcagcaagcagggtgtggaagtaggacattttcatgtcaggccacttctttccggagcggggttttgctatcacgttgtgaacttctgaagcggtgatgacgccgagccgtaatttgtgccacgcatcttccccctgttcgacagctctcacatcgatcccggtacgctgcaggataatgtccggtgtcatgctgccaccttctgctctgcggctttctgtttcaggaatccaagagcttttactgcttcggcctgtgtcagttctgacgatgcacgaatgtcgcggcgaaatatctgggaacagagcggcaataagtcgtcatcccatgttttatccagggcgatcagcagagtgttaatctcctgcatggtttcatcgttaaccggagtgatgtcgcgttccggctgacgttctgcagtgtatgcagtattttcgacaatgcgctcggcttcatccttgtcatagataccagcaaatccgaaggccagacgggcacactgaatcatggctttatgacgtaacatccgtttgggatgcgactgccacggccccgtgatttctctgccttcgcgagttttgaatggttcgcggcggcattcatccatccattcggtaacgcagatcggatgattacggtccttgcggtaaatccggcatgtacaggattcattgtcctgctcaaagtccatgccatcaaactgctggttttcattgatgatgcgggaccagccatcaacgcccaccaccggaacgatgccattctgcttatcaggaaaggcgtaaatttctttcgtccacggattaaggccgtactggttggcaacgatcagtaatgcgatgaactgcgcatcgctggcatcacctttaaatgccgtctggcgaagagtggtgatcagttcctgtgggtcgacagaatccatgccgacacgttcagccagcttcccagccagcgttgcgagtgcagtactcattcgttttatacctctgaatcaatatcaacctggtggtgagcaatggtttcaaccatgtaccggatgtgttctgccatgcgctcctgaaactcaacatcgtcatcaaacgcacgggtaatggattttttgctggccccgtggcgttgcaaatgatcgatgcatagcgattcaaacaggtgctggggcaggcctttttccatgtcgtctgccagttctgcctctttctcttcacgggcgagctgctggtagtgacgcgcccagctctgagcctcaagacgatcctgaatgtaataagcgttcatggctgaactcctgaaatagctgtgaaaatatcgcccgcgaaatgccgggctgattaggaaaacaggaaagggggttagtgaatgcttttgcttgatctcagtttcagtattaatatccattttttataagcgtcgactgtttcctgtgtgaaattgttatccgctcacaattccacacattatacgagccggaagcataaagtgtaaagcctggggtgcctaatgagtgagaattcggatctcgacgggatgttgattctgtcatggcatatccttacaacttaaaaaagcaaaagggccgcagatgcgacccttgtgtatcaaacaagacgattaaaaatcttcgttagtttctgctacgccttcgctatcatctacagagaaatccggcgttgagttcgggttgctcagcagcaactcacgtactttcttctcgatctctttcgcggtttccgggttatctttcagccaggcagtcgcattcgctttaccctgaccgatcttctcacctttgtagctgtaccacgcgcctgctttctcgatcagcttctcttttacgcccaggtcaaccagttcgccgtagaagttgataccttcgccgtagaggatctggaattcagcctgtttaaacggcgcagcgattttgttcttcaccactttcacgcgggtttcgctacccaccacgttttcgccctctttcaccgcgccgatacgacggatgtcgagacgaacagaggcgtagaatttcagcgcgttaccaccggtagtggtttccgggttaccgaacatcacaccaattttcatacggatctggttgatgaagatcagcagcgtgttggactgcttcaggttacccgccagcttacgcatcgcctggctcatcatacgtgccgcaaggcccatgtgagagtcgccgatttcgccttcgatttccgctttcggcgtcagtgccgccacggagtcaacgacgataacgtctactgcgccagaacgcgccagggcgtcacagatttccagtgcctgctcgccggtgtccggctgggagcacagcaggttgtcgatatcgacgcccagtttacgtgcgtagattgggtccagcgcgtgttcagcatcgataaacgcacaggttttaccttcacgctgcgctgcggcgatcacctgcagcgtcagcgtggttttaccggaagattccggtccgtagatttcgacgatacggcccatcggcagaccacctgccccaagcgcgatatccagtgaaagcgaaccggtagagatggtttccacatccatggaacggtcttcacccaggcgcatgatggagcctttaccaaattgtttctcaatctggcccagtgctgccgccaacgctttctgtttgttttcgtcgatagccatttttactcctgtcatgccgggtaataccggatagtcaatatgttctgttgaagcaattatactgtatgctcatacagtatcaagtgttttgtagaaattgttgccacaaggtctgcaatgcatacgcagtagcctgacgacgcaccgcatcacggtcgccgctgaagcatcccgccgggtaatgccttcaccgcgggcagtggcaaaagcaaaccagacggtgccgacaggcttctctagatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataatgattttattttgactgatagtgacctgttcgttgcaacaaattgataagcaatgccaagcttggcactggctgatcagctagctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatgctagcccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccg

SEQ ID NO: 150 - DNA, 3240 bp, Artificial Sequence
  Synthetic
ccacctgacgtctaagaaaccattattatcatgacattaacctataaaaataggcgtatcacgaggcagaatttcagataaaaaaaatccttagctttcgctaaggatgatttctggaattcgcggccgcatctagagtctcgagttgacagctagctcagtcctaggtataatgctagcctcgaggaaagaggagaaaggtctcaatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcgcgcatggtatggatgaactgtacagatgataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcaggaattcaaaaaaagcaccgactcggtgccactttttcaagttgataacggactagccttattttaacttgctatttctagctctaaaacggagacgagcgtctccactagtattatacctaggactgagctagctgtcaaggatccagcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 151 - DNA, 1756 bp, Artificial Sequence
  Synthetic
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgggatcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatctgtaggtcgttcgctccaagctggaaggatcttcacctagatccttttaaattaaaaatgaagttttaaatcaatctaaagtatatatgagtaaacttggtctgacagaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 152-165 - skipped (14 entries)

SEQ ID NO: 166 - DNA, 20 bp, Artificial Sequence
  Synthetic
gggaaacgggataatctgag

SEQ ID NO: 167 - DNA, 405 bp, Artificial Sequence
  Synthetic
gtgaatacaacgctgtttcgatggccggttcgtgtctactatgaagataccgatgccggtggtgtggtgtaccacgccagttacgtcgctttttatgaaagagcacgcacagagatgctgcgtcatcatcatttcagtcaacaggcgctgatggctgaacgcgttgcctttgtggtacgtaaaatgacggtggaatattacgcacctgcgcggctcgacgatatgctcgaaatacagactgaaataacatcaatgcgtggcacctctttggttttcacgcaacgtattgtcaacgccgagaatactttgttgaatgaagcagaggttctggttgtttgcgttgacccactcaaaatgaagcctcgtgcgcttcccaagtctattgtcgcggagtttaagcagtga

SEQ ID NO: 168 - DNA, 496 bp, Artificial Sequence
  Synthetic
gccatggccagagcgcgtggacgaggtcgtcgcgatctcaagtccgaaatcaacattgtaccgttgctggacgtactgctggtgctgttgctgatctttatggcgacagcgcccatcatcacccagagcgtggaggtcgatctgccagacgctactgaatcacaggcggtgagcagtaacgataatccgccagtgattgttgaagtgtctggtattggtcagtacaccgtggtggttgagaaagatcgtctggagcgtttaccaccagagcaggtggtggcggaagtgtccagccgtttcaaggccaacccgaaaacggtctttctgatcggtggcgcaaaagatgtgccttacgatgaaataattaaagcactgaacttgttacatagtgcgggtgtgaaatcggttggtttaatgacgcagcctatctaaacatctgcgtttcccttgcttgaaagagagcgggtaacaggcgaacagtttttggaaaccgaga

SEQ ID NO: 169 - DNA, 20 bp, Artificial Sequence
  Synthetic
tggtattggtcagtacaccg

SEQ ID NO: 170 - DNA, 415 bp, Artificial Sequence
  Synthetic
gcatcgtgccaatagccatgcgccggaagccgtagtggaaggggcgtcgcgtgcgatgcgtatttccatgaatcgtgaacttgaaaatctggaaacgcacattcctttcctcggtacggttggctccatcagcccgtatattggtctgtttggtacggtctgggggatcatgcacgcctttatcgccctcggggcggtaaaacaagcaacactgcaaatggttgcgcccggtatcgcagaagcgttgattgcgacggcaattggtttgttcgccgcaattccggcggtaatggcttataaccgcctcaaccagcgcgtaaacaaactggaactgaattacgacaactttatggaagagtttaccgcgattctgcaccgccaggcgtttaccgttagcgagagcaacaaggggtaa

SEQ ID NO: 171 - DNA, 474 bp, Artificial Sequence
  Synthetic
acatctgcgtttcccttgcttgaaagagagcgggtaacaggcgaacagtttttggaaaccgagagtgtcaaaggcaaccgaacaaaacgacaagctcaagcgggcgataattatttcagcagtgctgcatgtcatcttatttgcggcgctgatctggagttcgttcgatgagaatatagaagcttcagccggaggcggcggtggttcgtccatcgacgctgtcatggttgattcaggtgcggtagttgaacagtacaaacgtatgcaaagccaggaatcaagcgcgaagcgttctgatgagcagcgcaagatgaaggagcagcaggctgctgaagaactgcgtgagaaacaagcggctgaacaggaacgcctgaagcaacttgagaaagagcggttagcggctcaggagcagaaaaagcaggctgaagaagccgcaaaacaggccgagttaaagcagaagcaagcggaagaggc

SEQ ID NO: 172 - DNA, 20 bp, Artificial Sequence
  Synthetic
agaactgcgtgagaaacaag

SEQ ID NO: 173 - DNA, 496 bp, Artificial Sequence; identical to SEQ ID NO: 168
  Synthetic

SEQ ID NO: 174 - DNA, 500 bp, Artificial Sequence
  Synthetic
tcgcgatgttgactgttccgacggtcaacatcaggcaccggttgccacggggttctggtagttttgtgtattttagtttgttaacattctgctaaattatcgtgggccatcggtccagataagggagatatgatgaagcaggcattacgagtagcatttggttttctcatactgtgggcatcagttctgcatgctgaagtccgcattgtgatcgacagcggtgtagattccggtcgtcctattggtgttgttcctttccagtgggcggggcctggtgcggcacctgaagatattggcggcatcgttgctgctgacttgcgtaacagcggtaaatttaatccgttagatcgtgctcgtttgccacagcagccgggtagtgcgcaggaagtacaaccagctgcatggtccgcgctgggcattgacgctgtggttgtcggtcaggtcactccgaatccggatggttcttacaatgttgcttatcaacttgttgacactggcgg

SEQ ID NO: 175 - DNA, 20 bp, Artificial Sequence
  Synthetic
agtcaccgtagaaggtcacg

SEQ ID NO: 176 - DNA, 473 bp, Artificial Sequence
  Synthetic
tggtcgcagtaacaataccgaaccgacctggttcccggacagccagaacctggcatttacttctgaccaggccggtcgtccgcaggtttataaagtgaatatcaacggcggtgcgccacaacgtattacctgggaaggttcgcagaaccaggatgcggatgtcagcagcgacggtaaatttatggtaatggtcagctccaacggtgggcagcagcacattgccaaacaagatctggcaacgggaggcgtacaagttctgtcgtccacgttcctggatgaaacgccaagtctggcacctaacggcactatggtaatctacagctcttctcaggggatgggatccgtgctgaatttggtttctacagatgggcgtttcaaagcgcgtcttccggcaactgatggacaggtcaaattccctgcctggtcgccgtatctgtgataataattaattgaatagtaaaggaatcattgaa

SEQ ID NO: 177 - DNA, 471 bp, Artificial Sequence
  Synthetic
gagaattgcatgagcagtaacttcagacatcaactattgagtctgtcgttactggttggtatagcggccccctgggccgcttttgctcaggcaccaatcagtagtgtcggctcaggctcggtcgaagaccgcgtcactcaacttgagcgtatttctaatgctcacagccagcttttaacccaactccagcaacaactctctgataatcaatccgatattgattccctgcgtggtcagattcaggaaaatcagtatcaactgaatcaggtcgtggagcggcaaaagcagatcctgttgcagatcgacagcctcagcagcggtggtgcagcggcgcaatcaaccagcggcgatcaaagcggtgtggcggcatcaacgacgccgacagctgatgctggtactgcgaatgctggcgcgccggtgaaaagcggtgatgcaaacacggattacaatgcagctattgcgctggtgcag

SEQ ID NO: 178 - DNA, 20 bp, Artificial Sequence
  Synthetic
acgttcaggctgctaaagat

SEQ ID NO: 179 - DNA, 482 bp, Artificial Sequence
  Synthetic
aatacttcccggatgccaccatcctggcactgaccaccaacgaaaaaacggctcaccagttagtactgagcaaaggtgttgtgccgcagctggttaaagagatcacatctactgatgacttctaccgtctgggtaaagaactggctctgcagagcggtctggcacacaaaggtgatgtcgtagttatggtttctggtgcactggtaccgagcggcactactaacaccgcatctgttcacgtcctgtaatattgcttttgtgaattaatttgtatatcgaagcgccctgatgggcgctttttttatttaatcgataaccagaagcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttcccatcaaaaaaa

SEQ ID NO: 180 - DNA, 438 bp, Artificial Sequence
  Synthetic
cctgtgaagtgaaaaatggcgcacattgtgcgccatttttttgcctgctatttaccgctactgcgtcgcgcgtaacatattcccttgctctggttccccattctgcgctgactctactgaaggcgcattgctggctgcgggagttgctccactgctcaccgcaaccggataccctgcccgacgatacaacgctttatcgactaacttctgatctacagccttattgtctttaaattgcgtaaagcctgctggcagcgtgtacggcattgtctgaacgttctgctgttcttctgccgatagtggtcgatgtacttcaacataacgcatcccgttaggttccacggaatatttcaccggttcgttgatcactttcaccggtgttcccgtccgcacgctggagaataaagctttaatatccggtgcattcatgcgaataca

SEQ ID NO: 181 - DNA, 20 bp, Artificial Sequence
  Synthetic
caaaccgttcctctacaccg

SEQ ID NO: 182 - DNA, 417 bp, Artificial Sequence
  Synthetic
gacagccaggccatttgctcccgctcaccgagggacatcgacgggcgtggaaatgaccgctgtaatgtgcgactggcgggaaacgccaacataatgtgatgacgctgcggcagttcacgactccacggtaacaacgtgttagccagccgctgcacatcaacaatccgcccatctttgataatgtcgttctccagtggcaaccgccaccagcggtgcaataagcattcttttgcgctccgcacgatagcaaccgctaccgcttcttgctgttgtaaatgcaaaccaatttgccagttcttaaatgccattgtgatgatctccttatcacccgtcactctgacgggtatatcaatgcgtctggcttgcctttatactaccgcgcgtttgtttataaactgcccaaatgaaactaaatgg

SEQ ID NO: 183 - DNA, 460 bp, Artificial Sequence
  Synthetic
ttaaaaaggcgcttcggcgcctttttcagtttgctgacaaagtgcacttgtttatgccagatgcgggtgaacgcgttatccggccaacaaaaaattgaaaattcaataaattgcaggaacttcgtaagcctgataagcgttgtgcatcaggcaaacttcacgcatttacactcgcccctgccctttcaaccattcgcgcacgaggaacagcgcgctgacattacgcgcttcgttgaagtcaggggcttccagcaaatccatcatatgcgccagcggccagcgcacctgtggtagcggctctggctcatcgccttccagtgattccgggtagagatcttgcgctaccacgatattcattttgctggaaaagtaagacggtgccatgctgagcttcttcaaaaaagtcagatcgttcgcaccaaatccaacctcttcttttagctcgcggttagcggcttca

SEQ ID NO: 184 - DNA, 20 bp, Artificial Sequence
  Synthetic
cctgggactcatggccggat

SEQ ID NO: 185 - DNA, 516 bp, Artificial Sequence
  Synthetic
cccagcccttgtcgatgccgcgttcaggctagcttcgttcaaatccattaaagatatcagactactctcaggtacgtgagtaaggtaaaacccagttccaacgccaatatccagatggttgttacctaaatgttccagaaagtgtggaagaaggtgttcctttgtaggacatccccatgcaagccgatttgatactcccaaaacccaccagtcataaagctttagggtaagtggtgtgtaaattttagccccatcatctgtgttttttttcattagtttcaccgtgttatagttttatttgtgaattaaatcaattatagagatgaattacaaggggttaaatgatgccacggcataacgaatttgaatatattgggtcttgtgttgcggtaagaggttacacgccaggacgcaggttaaatgcttacaatattaggggatattgtttgcttttaacggtaaacaaattccccggggctatacaataccaccggggagaaaatctatttaacgttg

SEQ ID NO: 186 - DNA, 623 bp, Artificial Sequence
  Synthetic
aaaaggtctccattcaatcgttttaatgattgagtatgtattttttatatctaacttaatgagtcaattgtatattgccccactgtttatattttgtttaacattgaatttttgtcacaatgcgctgtcagttttttagtttaattgtctgtttgtgttttgtattgtggttttatgggctaattatttttttctttgtggagtttttatctattcaagtgccatgcctttagatgcatattagcgataatagcatgaggtttatcctcaattgctgtgttttttagtataaaaaagaggaacaaaactgagacacataaggcctcgcaatggcttgcaaggttttacatattttgaggtggtggaacgtgtgaacgcagagatggcgcggtaatttgttgatttaaaatgtcgttcttggagtttctaagtcgtgggctacaggttcgaatcctgcagggcgagccatttcctcgccatttatttgttcccttttacaaattacaccatcactttgttgtcgcggttttgctaaataattggaacacgtcagacgaataaaatgaaaacagaaattctgctaaacaattcatcttgccaggattttgattaggaaagtgaaa

SEQ ID NO: 187-199 - skipped (13 entries)

SEQ ID NO: 200 - DNA, 20 bp, Artificial Sequence
  Synthetic
tgtgtcgtcgttaactcgcc

SEQ ID NO: 201 - DNA, 409 bp, Artificial Sequence
  Synthetic
gccgcgcacgctgattttttatgaatctacccaccgtctgttagatagcctggaagatatcgttgcggtattaggcgaatcccgttacgtggttctggcgcgtgagctaaccaaaacctgggaaaccattcacggcgcgcccgttggcgagctgctggcgtgggtaaaagaagatgaaaaccgtcgcaaaggcgaaatggtgctgattgtcgaaggccataaagcgcaggaaaatgacttgcctgctgatgccctgcgcacgctggcgctgctacaggcagaactgccgctgaaaaaagcggcggcgctggccgcagaaattcacggcgtgaagaaaaatgcgctgtataagtatgcgctggagcagcagggagagtgattacaggcgtcgcagcaggaatagcgctac

SEQ ID NO: 202 - DNA, 405 bp, Artificial Sequence
  Synthetic
tcacgcatgtgaaatggttatgcagccataaaaatgaagccgggatagcggtggagactttagccgtgagaaaacgttctgtcgcatcctgttttccttcgccagtcaccagtaacaaaacttcgcgggcattgagaatatccttcaggcctaaggtgatcccacgagtcacgggacggtccgcggtttttaacatctcatgttgctgtgttctggcatcaagttgactgatatggcaggctggttgcaggctttctcccggttcgttcagcccaagatgaccgtttttccccaatccgagaacgcataaatccagaccgcctttgcgcgcaatcaggttcgttacccgttcgcactctgtctcatttatctcttcggagcgaaagctgatgagctggtcttcac

SEQ ID NO: 203 - DNA, 20 bp, Artificial Sequence
  Synthetic
cgctttttgaaaatacgcaa

SEQ ID NO: 204 - DNA, 477 bp, Artificial Sequence
  Synthetic
accagccgagattcaacaggttgttgcccgtcaggcgaccaatcaaaaactccattgagatgtagttaacatgtcgctgattcgccactggcttggcgaatggctgagcacgcagcatttcggccagtgcttcgctcactgccagccaccactggcgaggagtcatttcagccgcagaatttaagccataacgctgccactgacgtgaaagcgcttcctgaaattgcttatcgttaaaaataggttgtgacataggagttccacttttcttagattttcaacacaacgttatcgctagtttgccaggctcgatgttgaccttcctcatcctgcgggggattaggcagggaggagttgcggggatgagcaaggaaatgtgatctcgaccacttaaagctagtgcaatccacaggattagcagcaaatcaatgccataccgcgcagaaaatctgtatctaagtgcaaaaaatggccgtt

SEQ ID NO: 205 - DNA, 456 bp, Artificial Sequence
  Synthetic
cgtattttcaaaaagcggaaggtaactctataaattaagtaaaggagtgaaacagtttcacaagtaaaatatcaagtgtgctccatctcattcttaatagatttattaagatcatctttttagatggcactttcatcagaaatgaagaagaaacccttgcttaaatgaaactgatgaacataaggaaaataccgtacaacccagtattcacgctggatcagcgtcgttttaggtgagttgttaataaacatttggaattgtgacacagtgcaaattcagacacataaaaaaacgtcatcgcttgcattagaaaggtttctggccgaccttataaccattaattacgaagcgcaaaaaaaataatatttcctcattttccacagtgaagtgattaactatgctgattccgtcaaaattaagtcgtccggttcgactcgaccataccgtggttcgtga

SEQ ID NO: 206 - DNA, 20 bp, Artificial Sequence
  Synthetic
ccgtgacgttatccgcacca

SEQ ID NO: 207 - DNA, 357 bp, Artificial Sequence
  Synthetic
acgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatcc

SEQ ID NO: 208 - DNA, 442 bp, Artificial Sequence
  Synthetic
gtgacgctatcccgactcagtctgttctgaccatcacttctaacgttgtgtatggtaagaaaactggtaacaccccagacggtcgtcgtgctggcgcgccgttcggaccgggtgctaacccgatgcacggtcgtgaccagaaaggtgctgtagcgtctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgct

SEQ ID NO: 209 - DNA, 20 bp, Artificial Sequence
  Synthetic
gttatcgtgatgcgcattct

SEQ ID NO: 210 - DNA, 457 bp, Artificial Sequence
  Synthetic
ttccgcagccagcgattatcgcgccgcagcgcaacgcattctgccgccgttcctgttccactatatggatgggggggcatattctgaatacacgctgcgccgcaacgtggaagatttgtcagaagtggcgctgcgccagcgtattctgaaaaacatgtctgacttaagcctggaaacgacgctgtttaatgagaaattgtcgatgccggtggcgctaggtccggtaggtttgtgtggcatgtatgcgcgacgcggcgaagttcaggctgccaaagcagcagatgcgcatggcattccgtttactctctcgacggtttccgtttgcccgattgaagaagtggctccggctatcaaacgtccgatgtggttccagctttatgtgctgcgcgatcgcggctttatgcgtaacgccctggagcgagcaaaagccgcgggttgttcgacgctggttttcacc

SEQ ID NO: 211 - DNA, 441 bp, Artificial Sequence
  Synthetic
atccgcgatttctgggatggcccgatggtgatcaaagggatcctcgatccggaagatgcgcgcgatgcagtacgttttggtgctgatggaattgtggtttctaaccacggtggccgccagttagatggcgtactctcttctgctcgtgcactgcctgctattgcggatgcggtgaaaggtgatatcgccattctggcggatagcggaatacgtaacgggcttgatgtcgtgcgtatgattgcgctcggtgccgacaccgtactgctgggtcgtgctttcctgtatgcactggcaacagcgggccaggcgggtgtagctaatctgctaaatctgatcgaaaaagagatgaaagtggcgatgacgctgactggcgcgaaatcgattagcgaaattacgcaagattcgctggtgcaggggctgggtaaagagttgcctgcggca

SEQ ID NO: 212 - DNA, 20 bp, Artificial Sequence
  Synthetic
taacacccagcccgatgccc

SEQ ID NO: 213 - DNA, 357 bp, Artificial Sequence
  Synthetic
ttctgcatagcaggtgaggcaaatgagatttattcgccactacccagtatggatgagatctgaaaaagggagaggaaaattgcccggtagccttcactaccgggcgcaggcttagatggaggtacggcggtagtcgcggtattcggcgtgccagaaattatcatcaatggcctgttgcagagcttcggcagacgttttcaccgccacgccttgctgctgcgccattttgccaaccgcaaaggcaattgcgcgggagactttctgaatatctttcagttccggcagtaccagaccttcgccgttcaggaccagcggcgaatactgagcaagcatttcacttgccgacatcagcatctc

SEQ ID NO: 214 - DNA, 544 bp, Artificial Sequence
  Synthetic
gagattttcgcgcttctgcaccagtttggtctggaaaggcagcaggttcggcatcttgtcggtcagcaggccaaagcgatcgaccataaagactttctgccgcgccgcttcctcgcttaatccttcgcgctgggtctgggcgatgatcatttcggcaatgccgcatcccgctgaacctgcgccaaggaagacgatttttttctcgcttaactgaccacctgccgcgcggctggctgcgatcagtgtgccgactgttaccgccgcggtgccctgaatgtcatcgttaaaagaacaaatttcattgcgatagcggttaagtaacggcatcgcatttttttgtgcgaagtcttcaaactgcaacagtacgtccggccagcgttgtttcacagcctggataaattcatcaacaaattcatagtattcgtcgtcagtgatacgcggattacgccagcccatatacagcggatcgttaagcagctgttggttgttggtgccgacatccagcaccaccggaagggtataggccggactgatgccgccacag